SEQUENCE LISTING (8 sequences)

SEQ ID NO: 1 - DNA, 19 bp, Artificial
  Primer
cctcggccattcgtacatg

SEQ ID NO: 2 - DNA, 20 bp, Artificial
  primer
cgtagtaggacgggacgtgg

SEQ ID NO: 3 - DNA, 20 bp, Artificial
  Primer
tgtggatgtggtcttggtgg

SEQ ID NO: 4 - DNA, 19 bp, Artificial
  Primer
ataagcggttccctggcat

SEQ ID NO: 5 - DNA, 21 bp, Artificial
  Primer
gcacataaggccatcagctca

SEQ ID NO: 6 - DNA, 19 bp, Artificial
  Primer
tcgccagagagactggcaa

SEQ ID NO: 7 - DNA, 21 bp, Artificial
  Primer
tgcagtggcaaagtggagatt

SEQ ID NO: 8 - DNA, 20 bp, Artificial
  Primer
ttgaatttgccgtgagtgga

(19) United States
(12) Patent Application Publication
(10) Pub. No.: US 2010/0111927 A1
(43) Pub. Date: May 6, 2010

(54) Compositions Comprising Actinidia and Methods of Use Thereof

(76) Inventor: Sunyoung Kim, Seoul (KR)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 12/180,723

(22) Filed: Jul. 28, 2008

Related U.S. Application Data
(63) Continuation-in-part of application No. 11/945,153, filed on Nov. 26, 2007, now abandoned.
Application No. 11/945,153 is a continuation of application No. 11/697,565, filed on Apr. 6, 2007, now abandoned.
Application No. 11/697,565 is a continuation of application No. 11/518,380, filed on Sep. 8, 2006, now abandoned.
Application No. 11/518,380 is a continuation of application No. 11/362,606, filed on Feb. 23, 2006, now abandoned.
Application No. 11/362,606 is a continuation-in-part of application No. 10/646,145, filed on Aug. 22, 2003.
(60) Provisional application No. 60/656,839, filed on Feb. 25, 2005.
(60) Provisional application No. 60/656,838, filed on Feb. 25, 2005.
(60) Provisional application No. 60/405,295, filed on Aug. 23, 2002.

(51) Int. Cl. A61K 36/185; A61K 39/00; A61K 36/258; A61K 36/906; A61K 36/53; A61K 39/395; A61P 17/00; A61P 11/06; A61P 37/02; A61P 29/00; A61P 37/08

(52) U.S. Cl. 424/130.1; 424/777; 424/184.1; 424/773; 424/774; 424/775; 424/728; 424/756; 424/745

(57) ABSTRACT

Disclosed are novel preparations of Actinidia, and particularly species of hardy kiwifruit, as well as compositions comprising the same. Also disclosed is the use of these preparations of Actinidia to prevent and/or treat a variety of diseases in which regulation of the immune response is effective, including both allergic and non-allergic inflammatory disease, viral infection, and cancer. Methods related to making and using these compositions are also described.

### CROSS-REFERENCE TO RELATED APPLICATIONS

This application is a continuation-in-part of U.S. patent application Ser. No. 11/945,153, filed Nov. 26, 2007, which is a continuation of U.S. patent application Ser. No. 11/697,565, filed Apr. 6, 2007, which is a continuation of U.S. patent application Ser. No. 11/518,380, filed Sep. 8, 2006, which is a continuation of U.S. patent application Ser. No. 11/362,606, filed Feb. 23, 2006, which claims the benefit of priority under 35 U.S.C. §119(e) from U.S. Provisional Application No. 60/656,839, filed Feb. 25, 2005 and from U.S. Provisional Application No. 60/656,838, filed Feb. 25, 2005. This application is also a continuation-in-part of U.S. patent application Ser. No. 10/646,145, filed Aug. 22, 2003, which claims the benefit of priority under 35 U.S.C. §119(e) from U.S. Provisional Application No. 60/405,295, filed Aug. 23, 2002. U.S. patent application Ser. No. 10/646,145 and U.S. Provisional Application No. 60/405,295 are incorporated herein by reference in their entirety.

### REFERENCE TO SEQUENCE LISTING

This application contains a Sequence Listing submitted on a compact disc, in duplicate. Each of the two compact discs, which are identical to each other pursuant to 37 CFR §1.52(e)(4), contains the following file: “Sequence Listing”, having a size in bytes of 2 KB, recorded on 23 Feb. 2006. The information contained on the compact disc is hereby incorporated by reference in its entirety pursuant to 37 CFR §1.77(b)(4).

### FIELD OF THE INVENTION

The present invention relates to Actinidia, and particularly species of hardy kiwifruit, and various fractions and preparations thereof, as well as compositions comprising the same, all having the ability to prevent and/or treat a variety of diseases in which regulation of the immune response is effective, including both allergic and non-allergic inflammatory disease, viral infection, and cancer. Methods related to making and using these compositions are also described.

### BACKGROUND OF THE INVENTION

Diseases involving inflammation are characterized by the influx of certain cell types and mediators, the presence of which can lead to tissue damage and sometimes death. Diseases involving inflammation are particularly harmful when they afflict certain organs and systems, such as the respiratory system, which can result in obstructed breathing, hypoxemia, hypercapnia and lung tissue damage. In other diseases or conditions, the development of certain types of inflammation can be an important component in controlling the disease, such as in a viral infection, although damage to tissues in the area of infection is still a risk.

Allergic diseases are mediated in part by immunoglobulin E (IgE), while the type-2 T helper (Th2) cells, mast cells and eosinophils have also been shown to play important roles in the disease process (Maggi E., Immunotechnology, 3:233-244, 1998; Pawankar R., Curr. Opin. Allergy Clin. Immunol., 1:3-6, 2001; Vercelli D., Clin. Allergy Immunol., 16:179-196, 2002). Circulating IgE binds to two isoforms of IgE receptors: high-affinity IgE receptors (FcεRI) present on the surface of mast cells and basophils, and low affinity IgE receptors (FcεRII or CD23) present on the surfaces of lymphocytes, eosinophils, platelets and macrophages. It is believed that an important factor governing the pathogenesis of allergic disorders is the cross-linkage of IgE receptors on mast cells, after encountering allergen and the consequent degranulation of mast cells. The molecules released by mast cells include histamine, heparin, proteases and free radicals, which mediate a variety of biological effects including vasodilation, intestinal and/or bronchial smooth muscle contraction, mucous secretion and local proteolysis. Following an initial immediate reaction of the mast cells, an influx of eosinophils, basophils and lymphocytes occurs 6-24 hours later. This late-phase response can lead to chronic tissue inflammation in tissues that are continuously exposed to antigens.

The degranulation of IgE-dependent mast cells and the accumulation of eosinophils in the sites of inflammation are considered to result from the unbalanced overactivation of Th2 cells and consequently the Th2-mediated overproduction of IgE (Abbas et al., 1991, Nature 383:787-93; Vercelli, 2001, Curr Opin Allergy Clin Immunol 2001, 1:61-5). The representative cytokines of Th2 cells, such as IL-4, IL-5, IL-10 and IL-13, are known to play important roles in these reactions. Furthermore, Th1-mediated cytokines such as IFN-γ and IL-12 were reported to negatively regulate the Th2 pathways. For example, IFN-γ induces the isotype-switching to IgG2a in B cells, while IL-12 converts the already established Th2 response to Th1 dominance in certain situations (Umetsu and DeKruyff, 1997, J Allergy Clin Immunol 100:1-6; Coffman and Carty, 1986, J Immunol 136:949-54; Gavett et al., 1995, J Exp Med 182:1527-36). Various cellular transcription factors, such as GATA3 and T-bet, control the differentiation of Th1 and Th2 cells and the production of cytokines in these cells (Lee et al., 2000, J Exp Med 192:105-15; Ting et al., 1996, Nature 384:474-8; Lighvani et al., 2001, Proc Natl Acad Sci USA 98:15137-42; Szabo et al., 2000, Cell 100:655-69).

Allergic diseases such as anaphylaxis, allergic rhinitis, asthma, atopic dermatitis, food allergies and urticaria, afflict up to 20% of the population in many countries and are increasing in prevalence (Wuthrich B., Int. Arch. Allergy Appl. Immunol. 90:3-10, 1989).

For example, asthma is a significant disease of the lung which affects millions of people worldwide. Asthma is typically characterized by periodic airflow limitation and/or hyperresponsiveness to various stimuli which results in excessive airways narrowing. Other characteristics can include inflammation of airways, eosinophilia and airway fibrosis. Heightened airway responsiveness is thought to result from a complex inflammatory cascade involving several cell types, including T lymphocytes and eosinophils. In allergic asthma, Th2 cytokines predominate over Th1 cytokines.

Atopic dermatitis (AD) is a chronic and relapsing inflammatory skin disease characterized by pruritic and eczematous skin lesions, along with elevated IgE levels. The incidence of AD appears to be increasing worldwide in infants and children. The skin lesions of AD patients are characterized by the infiltration of inflammatory cells including T lymphocytes, monocytes/macrophages, eosinophils and mast cells. These cells are involved in the pathogenesis and the development of AD through the release of various cytokines and chemokines such as IL-4, IL-5, IL-10, IL-13, eotaxin, and TARC. Among many cell types, Th2 cells producing IL-4, IL-5, IL-10, and IL-13 play a critical role in the initiating phase of the disease progression (Leung, 1997, Clin Exp Immunol 107(suppl. 1):25-30). IL-4 and IL-13 act as major isotype inducers switching to IgE, and IL-5 induces the activation of eosinophils, which secretes various chemokines such as eotaxin. IL-10 produced by monocytes/macrophages as well as Th2 cells augments the production of TARC, which is a Th2-specific chemokine and known to be overexpressed in AD lesions. Although Th1-type cytokines, such as IFN-γ, are also found in the skin lesions of AD during the later stage of the disease, the development of AD is thought to be caused primarily by the overproduction of Th2-mediated cytokines/chemokines and IgE, as well as the defective production of IFN-γ and IL-12 (Jonathan et al., 1999, J Clin Invest 103:1103-11; Christian et al., 1999, J Clin Invest 104:1097-105; Tomomi et al., 2001, J Allergy Clin Immunol 107:353-8; Weilie et al., 2002, J Clin Invest 109:621-8). However, the exact mechanisms associated with the hyperproduction of IgE and the unbalance of Th1/Th2 responses has not been clarified.

Because a bulk of evidence has suggested that the Th1 and Th2 types of reactions are reciprocally regulated in vivo, the modulation of Th1/Th2 has been thought to be a rational strategy for developing the therapeutics of allergic diseases (Kato et al., 1999, J Immunol 162:7470-9). For example, recombinant cytokines such as IL-12 and IFN-γ or cytokine receptor antagonists to IL-4 and IL-5 have been tested for their ability to control the balance between Th1 and Th2 responses (Hofstra et al., 1998, J Immunol 161:5054-60; Tomkinson et al., 2001, J Immunol 166:5792-800). However, the direct administration of these agents often causes undesirable side effects.

Leukotrienes are also associated with a variety of diseases associated with inflammation, and particularly, with allergic inflammation. Leukotrienes are derived from arachidonic acid, the precursor of prostaglandins. There are two families of leukotrienes. The first group acts primarily in conditions in which inflammation is dependent on neutrophils, such as cystic fibrosis, inflammatory bowel disease, and psoriasis. The second group (cysteinyl-leukotrienes) is concerned primarily with eosinophil- and mast cell-induced bronchoconstriction in asthma. They bind to highly selective receptors on bronchial smooth muscle and other airway tissue (O'Byrne et al., Annals of Internal Medicine 1997; 127:472-80). Leukotrienes are also known to be important in the pathophysiology of allergic rhinitis, chronic urticaria and atopic dermatitis or eczema. Leukotriene antagonists, including both leukotriene synthesis inhibitors and cysteinyl-leukotriene receptor antagonists, are useful to specifically inhibit the production or actions of these inflammatory mediators.

The hypothesis that reducing serum IgE levels could improve allergic symptoms has been demonstrated by clinical trials using chimeric anti-IgE antibody (CGP-51901) and recombinant humanized monoclonal antibody (rhuMAB-E25) (Fahy et al., Am. J. Respir. Crit. Care. Med., 155:1828-1834, 1997). Diacyl benzimidazole analogs and bacterial polysaccharides that inhibit IgE synthesis and secretion have been described in U.S. Pat. No. 6,369,091 and U.S. Patent Publication No. 20020041885, respectively.

Korea Patent Application No. 92-11752 disclosed an anti-inflammatory, anti-allergic and anti-rheumatic drug comprising bioflavonoid such as 4′-O-methyl ochnaflavone isolated from Lonicera japonica, which shows efficacy in the treatment of various symptoms associated with allergy or inflammation. Korea Patent Registration No. 100744 disclosed an anti-inflammatory, anti-allergic and anti-rheumatic drug comprising several bioflavonoid compounds isolated from the leaves of Ginko biloba. Several Oriental medicinal recipes comprising Siegesbeckia glabrescens have been reported to have IgE-reducing activity (Kim et al., Phytother. Res., 15:572-576, 2001). Furthermore, many medicinal herbs have been found to be rich sources of histamine release inhibitors or anti-inflammatory compounds.

Other conventional drugs for the treatment of allergic disorders include anti-histamines, steroidal or non-steroidal anti-inflammatory drugs and leukotriene antagonists. These agents, however, have the potential of serious side effect, including, but not limited to, increased susceptibility to infection, liver toxicity, drug-induced lung disease, and bone marrow suppression. Thus, such drugs are limited in their clinical use for the treatment inflammation, and particularly allergic inflammation. The use of anti-inflammatory and symptomatic relief agents is a serious problem because of their side effects or their failure to attack the underlying cause of an inflammatory response. There is a continuing requirement for less harmful and more effective agents for treating inflammation. Thus, there remains a need for new products with lower side effect profiles, less toxicity and more specificity for the underlying cause of the inflammation.

Finally, the elicitation of an immune response that favors the activation of Th1-type T cells, the production of IgG2a, and the production of the associated Th1 type cytokines (e.g., IFN-γ, IL-6, IL-12, IL-1), is in contrast to the immune response associated with allergic inflammation discussed above. This type of immune response can have strong, systemic, anti-tumor and anti-viral properties. There is a continued need in the art for providing products with such properties.

### SUMMARY OF THE INVENTION

One embodiment of the present invention relates to a method to regulate an immune response in a mammal. The method includes administering a hardy kiwifruit preparation to the mammal in an amount sufficient to regulate an immune response in the mammal.

Another embodiment of the present invention relates to a method to treat a disease or condition that is associated with dysregulation of immune function. The method includes administering to a mammal a combination of hardy kiwifruit or a preparation thereof and an agent selected from: an antihistamine, an antibody, an antibiotic, a cyclosporin, an antimycotic, a respiratory function controller, an analgesic, a β-agonists, a leukotriene modifier, a cytokine antagonist, a cytokine receptor antagonist, a phosphodiesterase inhibitor, sodium cromoglycate, nedocrimil, caffeine, theophylline, carbobenzoxy beta-alanyl taurine, and an inhibitor of T cell function.

Yet another embodiment of the present invention relates to a method to treat a disease or condition that is associated with dysregulation of immune function. The method includes administering to a mammal a combination of a hardy kiwifruit or a preparation thereof and an agent selected from: fatty acids; polyketides; citric acid; fumaric acid; guaiacol; methylsulfonylmethane (MSM); aromatic amino acids; phenylpropanoids; terpenoids; alkaloids; corrins; porphyrins; linear peptides; cyclic peptides; depsipeptides; amino acids derivatives; nucleosides; nucleotides; carbohydrates; proteins; cells; cell fragments; herbal preparations; spices; minerals; sterilizers; seasonings; vitamins; and electrolytes. Fatty acids can include, but are not limited to, conjugated linolenic acid, eicosapentaenoic acid, docosahexaenoic acid, γ-linolenic acid, α-linolenic acid, dihomo-γ-linolenic acid, and stearidonic acid.

Another embodiment of the present invention relates to a method to treat a disease or condition that is associated with dysregulation of immune function. The method includes administering to a mammal a combination of hardy kiwifruit or a preparation thereof and an agent selected from: probiotics; bacterial cell walls and fragments; whey protein; taurine; alanine; fatty acids; fatty acid esters; monoglycerides; diglycerides; triglycerides; inositol; turmeric; curcumin; rosemary; rosemarinic acid; methylsulfonylmethane (MSM); ginseng; ginger; proanthocyanidin; and β-carotene. Fatty acids can include, but are not limited to, conjugated linolenic acid, eicosapentaenoic acid, docosahexaenoic acid, γ-linolenic acid, α-linolenic acid, dihomo-γ-linolenic acid, and stearidonic acid.

In any of the above-described embodiments of the invention, the hardy kiwifruit preparation can be provided in an amount sufficient to: regulate a Th2 and a Th1 immune response in the mammal; regulate the amount of an antibody isotype produced by the mammal selected from IgE, IgG2a, and IgG1; decrease the production and/or levels of at least one Th2 cytokine in the mammal or to increase the level of at least one Th1 cytokine in the mammal; decrease the level of or production of at least one leukotriene in the mammal; or decrease the level of expression of a transcription factor selected from the group consisting of: GATA-3, T-bet and NFATc2 in the mammal. In one aspect, the mammal has or is at risk of developing a condition in which enhancement of a Th1 response and/or suppression of a Th2 response is desirable. In one aspect, the mammal has or is at risk of developing an allergic disease (e.g., an allergic disease regulated by leukotrienes) or non-allergic inflammatory disease. Allergic diseases include, but are not limited to, asthma, atopic dermatitis, food allergy, allergic rhinitis, and chronic urticaria. In another aspect, the mammal has or is at risk of developing a viral infection or a cancer.

In one aspect of any of the above-described embodiments of the invention, the hardy kiwifruit is selected from the group consisting of: Actinidia arguta, Actinidia kolomikta and Actinidia polygama, with Actinidia arguta being one preferred aspect. In one aspect, the hardy kiwifruit preparation is selected from: a fruit extract or concentrate, a leaf extract or concentrate, a stem extract or concentrate, a bark extract or concentrate, a root extract or concentrate, fresh fruit, crushed fruit, boiled fruit, cooked fruit, pressed fruit, condensed fruit, dried fruit, a hardy kiwifruit juice concentrate, a preparation produced by extraction of fruit in water having a temperature of from 0° C. to about 80° C.; a preparation produced by direct extraction of a water soluble concentrate of hardy kiwifruit with ethyl acetate, a preparation produced by extraction of hardy kiwifruit in distilled water, and a preparation produced by sequential extraction in water, chloroform and ethyl acetate. In one aspect, the hardy kiwifruit preparation is an extract or concentrate prepared from a part of the hardy kiwifruit selected from: the leaf, the stem, the bark, the root, and any combination thereof. In one aspect, the hardy kiwifruit preparation is produced by a method that includes a step of drying the fruit. In one aspect, the hardy kiwifruit preparation is produced by extraction of fruit in room temperature water.

In one aspect of any of the above-described embodiments of the invention, the hardy kiwifruit preparation is provided in a composition in an amount of between about 0.01% and about 95% by weight based on the total weight of the composition. In another aspect, the step of administering comprises administering the hardy kiwifruit preparation with a carrier, adjuvant, or diluent to the mammal. In one aspect, the step of administering comprises providing the hardy kiwifruit preparation to the mammal as a tablet, a powder, an effervescent tablet, an effervescent powder, a capsule, a liquid, a suspension, a granule or a syrup.

In one aspect of any of the above-described embodiments of the invention, the step of administering comprises providing the hardy kiwifruit preparation to the mammal in a health food. Health foods include, but are not limited to, fine bakery wares, bread, rolls, breakfast cereals, processed cheese, unprocessed cheese, condiments, dairy products, puddings, gelatin desserts, carbonated drinks, teas, powdered beverage mixes, processed fish products, fruit-based drinks, vegetable-based drinks, chewing gum, hard confectionery, frozen dairy products, processed meat products, nut-based spreads, pasta, processed poultry products, gravies and sauces, potato chips, vegetable chips, crisps, chocolate, cookies, candy, licorice, ice creams, dehydrated foods, cut food products, processed food products, spices, alcoholic beverages, noodles, fermented foods, soups, soup mixes, soya based products, vegetable oil-based spreads, and vegetable-based drinks.

In one aspect of any of the above-described embodiments of the invention, the step of administering comprises applying a cosmetic composition comprising the hardy kiwifruit preparation to the mammal. The cosmetic composition can be provided in a form including, but not limited to, lotion, cream, essence, toner, emulsion, pack, soap, shampoo, rinse, cleanser, body washing solution, washing solution or treatment.

In another aspect of any of the above-described embodiments of the invention, the step of administering comprises providing the hardy kiwifruit preparation to the mammal in a food additive.

In another aspect of any of the above-described embodiments of the invention, the method further comprises administering to the mammal an agent selected from: fatty acids (e.g., conjugated linolenic acid, eicosapentaenoic acid, docosahexaenoic acid, γ-linolenic acid, α-linolenic acid, dihomo-γ-linolenic acid, and stearidonic acid); polyketides; organic acids; small organic compounds; aromatic amino acids; phenylpropanoids; terpenoids; alkaloids; corrins; porphyrins; linear peptides; cyclic peptides; depsipeptides; amino acids derivatives; nucleosides; nucleotides; carbohydrates; proteins; cells; cell fragments; herbal preparations; spices; minerals; sterilizers; seasonings; vitamins; and electrolytes.

In another aspect of any of the above-described embodiments of the invention, the method further comprises administering to the mammal an agent selected from: probiotics; bacterial cell walls and fragments; whey protein; taurine; alanine; fatty acids (e.g., conjugated linolenic acid, eicosapentaenoic acid, docosahexaenoic acid, γ-linolenic acid, α-linolenic acid, dihomo-γ-linolenic acid, and stearidonic acid); fatty acid esters; monoglycerides; diglycerides; triglycerides; inositol; turmeric; curcumin; rosemary; rosemarinic acid; methylsulfonylmethane (MSM); ginseng; ginger; proanthocyanidin; and β-carotene.

In another aspect of any of the above-described embodiments of the invention, the method further includes administering to the mammal a different Actinidia species preparation, including, but not limited to, A. chenensis, A. deliciosa, A. arguta, A. polygama, and A. kolomikta.

In yet another aspect of any of the above-described embodiments of the invention, the method further includes administering to the mammal at least one additional active compound for regulating an immune response in a mammal, and particularly, for treating or preventing allergic disease in a mammal.

Another embodiment of the present invention relates to a composition for regulating an immune response in a mammal, comprising a hardy kiwifruit preparation and at least one additional active compound for regulating an immune response in a mammal. The hardy kiwifruit preparation can include any of the above-described hardy kiwifruit preparations. In one aspect, the additional active compound is selected from: antihistamines, antibodies, antibiotics, cyclosporins, antimycotics, respiratory function controllers, analgesics, β-agonists, leukotriene modifiers, cytokine or cytokine receptor antagonists, phosphodiesterase inhibitors, sodium cromoglycate, nedocrimil, caffeine, theophylline, carbobenzoxy beta-alanyl taurine, and inhibitors of T cell function. In another aspect, the additional active compound is selected from: fatty acids; polyketides; citric acid; fumaric acid; guaiacol; methylsulfonylmethane (MSM); aromatic amino acids; phenylpropanoids; terpenoids; alkaloids; corrins; porphyrins; linear peptides; cyclic peptides; depsipeptides; amino acids derivatives; nucleosides; nucleotides; carbohydrates; proteins; cells; cell fragments; herbal preparations; spices; minerals; sterilizers; seasonings; vitamins; and electrolytes. In another aspect, the additional active compound is selected from: probiotics; bacterial cell walls and fragments; whey protein; taurine; alanine; fatty acids; fatty acid esters; monoglycerides; diglycerides; triglycerides; inositol; turmeric; curcumin; rosemary; rosemarinic acid; methylsulfonylmethane (MSM); ginseng; ginger; proanthocyanidin; and β-carotene. Fatty acids can include, but are not limited to, conjugated linolenic acid, eicosapentaenoic acid, docosahexaenoic acid, γ-linolenic acid, α-linolenic acid, dihomo-γ-linolenic acid, and stearidonic acid.

The composition of the invention can be provided in a form including, but not limited to, a pharmaceutical composition, a health food, a food additive, and a cosmetic. In one aspect, the composition is for the treatment of a disease or condition that is associated with leukotriene production or activity. In one aspect, the composition is for the treatment of a disease or condition selected from: atopic dermatitis, asthma, food allergy, allergic rhinitis, and chronic urticaria.

### BRIEF DESCRIPTION OF THE DRAWINGS

FIG. 1 shows the inhibitory activity of various preparations from A. arguta on the production of IgE in U266B1 cells. Results were calculated as the percentage of IgE level produced from U266B1 cells treated with LPS only, from three-independent experiments.

FIG. 2 shows the dose-dependent effects of PG102T and PG102E on IL-4 production in OVA-stimulated splenocytes. Specific activity of PG102T and PG102E was determined from the IC₅₀ value.

FIGS. 3A-3C illustrate the effects of PG102T and PG102E on the number of IL-4 or IFN-γ-producing T cells (FIG. 3A) and IgE-producing B cells (FIG. 3B) and IgE biosynthesis within B cells (FIG. 3C). Data are the mean of percentages of each population from three independent experiments. *, P<0.05 vs DW-treated mice.

FIGS. 4A-4B show the effects of PG102T and PG102E on the expression of GATA3, T-bet and NFATc2, by Western blot (FIG. 4A) and quantitative real-time PCR analysis (FIG. 4B). Results are expressed as mean±SEM from three independent experiments. *, P<0.05 and **, P<0.01 vs. DW-treated mice. β-actin and GAPDH were used as a loading control.

FIGS. 5A-5B show the effects of PG102T and PG102E on the development of dermatitis in NC mice using dermatitis index (FIG. 5A) and scratching incidence (FIG. 5B). Values are expressed as means±SEM of 5-6 animals. *, P<0.05; **, P<0.01, vs. DW-treated mice.

FIGS. 6A-6C show the effects of PG102T and PG102E on the plasma levels of IgE (FIG. 6A), IgG1 (FIG. 6B) and IgG2a (FIG. 6C) in NC mice. Values are expressed as mean±SEM of 5 animals. *, P<0.05; **, P<0.01, vs. DW-treated mice.

FIGS. 7A-7B show the effects of PG102T and PG102E on the number of total leukocytes and eosinophils (FIG. 7A) and the production of eotaxin and TARC (FIG. 7B) in peripheral blood. Values are expressed as mean±SEM of 5 animals. *, P<0.05; **, P<0.01, vs. DW-treated mice.

FIGS. 8A-8B show the effects of PG102T and PG102E on the skin lesions in NC mice from back skin (FIG. 8A) and face skin (FIG. 8B). *, P<0.05; **, P<0.01, vs. DW-treated mice.

FIGS. 9A-9B show the effects of PG102T and PG102E on the expression of IL-4, IL-5, eotaxin, TARC, GATA3 and pSTAT6 in the skin lesions, as measured by ELISA (FIG. 9A) and Western blot (FIG. 9B). Values are expressed as mean±SEM of 5 animals. *, P<0.05; **, P<0.01, vs. DW-treated mice. Numbers in parentheses indicate percent activity relative to DW-treated mice.

FIG. 10 is a schematic of the process used in order to produce larger amounts of PG102T; this frozen or otherwise dried kiwifruit concentrate is also referred to as FD001 (FG refers to food grade carrier).

FIG. 11 shows the effect of three doses (0.25, 1.0, and 10 mg/mL) of FD001 (PG102T) on the relative degree of production of the cytokines IL-4, IL-5, IL-10, IL-13, and IFN-γ by OVA-stimulated mouse splenocytes following 3 days exposure in vitro. Cytokine levels were measure by ELISA. Each point represents the average of data from splenocytes of ten individual mice.

FIG. 12 shows the effect of three doses (0.25, 1.0, and 10 mg/mL) of an ethyl acetate (EtOAc) extract of FD001 (PG102T) on the relative degree of production of the cytokines IL-4, IL-5, IL-10, IL-13, and IFN-γ by OVA-stimulated mouse splenocytes following 3 days exposure in vitro. Cytokine levels were measure by ELISA. Each point represents the average of data from splenocytes of ten individual mice.

FIG. 13 shows the effect of three doses (0.25, 1.0, and 10 mg/mL) of an A. arguta fruit juice concentrate on the relative degree of production of the cytokines IL-4, IL-5, IL-10, IL-13, and IFN-γ by OVA-stimulated mouse splenocytes following 3 days exposure in vitro. Cytokine levels were measure by ELISA. Each point represents the average of data from splenocytes of ten individual mice.

FIGS. 14A and 14B indicate the activity of three doses of known immunosuppressant compounds on the relative degree of production of IL-13, and IFN-γ by OVA-stimulated mouse splenocytes following 3 days exposure in vitro. Cyclosporin was tested at 0.0083, 0.083, and 4.15 μM and dexamethasone was tested at 0.01, 0.1, and 1 μM (FIG. 14A) with each point representing the average of data from splenocytes of ten individual mice. Quercetin was tested at 1.0, 10, and 25 μM with each point representing the average of data from splenocytes of two individual mice (FIG. 14B). Cytokine levels were measure by ELISA.

FIGS. 15A and 15B show the effect of three doses (1.0, 3.0, and 10 mg/mL) of FD001 (PG102T), an ethyl acetate (EtOAc) extract of FD001, and the aqueous remainder from this process on the relative degree of production of IL-13 (FIG. 15A) and IFN-γ (FIG. 15B) by OVA-stimulated mouse splenocytes following 3 days exposure in vitro. Cytokine levels were measure by ELISA. Each point represents the average of data from splenocytes of eight individual mice.

FIGS. 16A and 16B show the effect of three doses (1.0, 3.0, and 10 mg/mL) of FD001 (PG102T) and a powdered form of FD001 (created for use in capsules) on the relative degree of production of IL-13 (FIG. 16A) and IFN-γ (FIG. 16B) by OVA-stimulated mouse splenocytes following 3 days exposure in vitro. Cytokine levels were measure by ELISA. Each point represents the average of data from splenocytes of eight individual mice.

FIGS. 17A and 17B show the effect of alternative preparations of A. arguta on the relative degree of production of IL-13 (FIG. 17A) and IFN-γ (FIG. 17B) by OVA-stimulated mouse splenocytes following 3 days exposure in vitro. Three doses (1.0, 3.0, and 10 mg/mL) each of FD001 (PG102T), a fruit juice concentrate, an extract prepared by boiling of the fresh fruit in water, and a room temperature water extract of the fruit were tested. Cytokine levels were measure by ELISA. Each point represents the average of data from splenocytes of eight individual mice.

FIGS. 18A and 18B show the effect of preparations of alternative plant parts of A. arguta on the relative degree of production of IL-13 (FIG. 18A) and IFN-γ (FIG. 18B) by OVA-stimulated mouse splenocytes following 3 days exposure in vitro. Three doses (1.0, 3.0, and 10 mg/mL) each of a fruit juice concentrate, individual extracts prepared by boiling of the bark, root, or stem in H₂O, and FD001 were tested. Cytokine levels were measured by ELISA. Each point represents the average of data from splenocytes of eight individual mice.

### DETAILED DESCRIPTION OF THE INVENTION

The present inventors have previously discovered that the hardy kiwifruit, and particularly certain extracts/concentrates derived therefrom, generally referred to herein as PG102, increase serum levels of Th1 cytokines and IgG2a, reduce serum levels of Th2 cytokines and IgE, inhibit histamine release from mast cells, and suppress allergic inflammatory reactions, including in an allergen-sensitized murine model of allergic inflammation and airway hyperresponsiveness, as well as in a rat paw edema assay (see U.S. Patent Publication No. 2004/0037909, supra). PG102 is orally active.

The present inventors provide further evidence herein that two specific extracts prepared from Actinidia arguta, denoted PG102T and PG102E (described in detail in Examples 1 and 2 below), exhibit inhibitory activity on the production of IgE as well as the ability to regulate selective Th1 and Th2 cytokines. This regulatory activity is most likely achieved by the regulation of cellular transcription factors, GATA-3, T-bet and NFATc2. The present inventors' data indicates the great potential of PG102T and PG102E as natural immune modulators of the Th1 and Th2 pathways, and ultimately as anti-allergy agents. The efficacy of these extracts is demonstrated herein in vivo in a model for allergic inflammation associated with respiratory conditions, and in a model for atopic dermatitis.

The present inventors also describe herein other preparations of hardy kiwifruit, including preparations of whole fruit, stem, root, bark, new extracts, concentrates, juices, dried preparations and non-extracted preparations, and demonstrate that such hardy kiwifruit preparations have similar properties with regard to their ability to regulate Th1 and Th2 cytokines and are believed to therefore have similar anti-allergy properties.

In addition, the present inventors have demonstrated that hardy kiwifruit preparations of the present invention reduce levels of leukotrienes and blunt production of leukotrienes in vivo, indicating that the compositions of the present invention can be used to treat leukotriene-mediated diseases, including, but not limited to, atopic dermatitis, asthma, food allergy, allergic rhinitis, and chronic urticaria.

The identification of the hardy kiwifruit and preparations thereof as enhancers of Th1 responses makes these agents particularly attractive for the treatment of diseases and conditions that would benefit from such a response, including, but not limited to, viral infection and cancer.

The invention includes the use of any members of the family, Actinidiaceae, and particularly any members of the genus Actinidia, including, but not limited to, the common kiwi known as A. chinensis or A. deliciosa, and the hardy kiwifruit known as A. arguta, A. polygama, or A. kolomikta, to provide compositions of kiwifruit with immune regulatory activity. Preferably, the kiwi is a hardy kiwifruit. The term “kiwifruit” can be used herein to generically refer to any member of the genus Actinidia, and includes the members of the hardy kiwifruit as discussed above, as well as members of the common kiwi, also as discussed above.

More than 30 species belonging to Actinidiaceae have been reported. Among those, the fruit of A. chinensis or A. deliciosa have been named “kiwi” and are popular edible fruits. Reference herein to “common kiwi” or “common kiwifruit” is intended to refer to A. chinensis or A. deliciosa. According to the present invention, reference to “hardy kiwifruit” or “hardy kiwi” refers to any of A. arguta, A. polygama, and A. kolomikta, or another species of the Actinidia genus related thereto that has the bioactive properties of A. arguta, A. polygama, and A. kolomikta as initially described in U.S. Patent Publication No. 2004/0037909, and as further described in PCT Publication No. WO 2006/093793, or as further described herein, particularly with regard to the anti-allergy properties and the immune response/cytokine/leukotriene-regulatory properties of the fruit or preparations thereof. Reference generally to a “kiwifruit” or “kiwifruit preparation” intends to refer to any species of Actinidia, including common and hardy kiwifruit.

Actinidia arguta, Actinidia polygama, and Actinidia kolomikta belonging to Actinidiaceae, are naturally distributed in Siberia, the northern area of China, Japan, North and South Korea. A. arguta and other fruit of the same genus (e.g., A. polygama, and A. kolomikta) have been used as materials of Chinese medicine named as ‘mihudo’ to treat liver disease, gastrointestinal disease and urogenital lithiasis without toxicity (Seoul National University Natural Products Science, Tradi-Medi Data Base, dongbang media Co. Ltd. 1999). As discussed above, U.S. Patent Publication No. 2004/0037909, discloses the treatment and prevention of allergic disease and non-allergic inflammatory disease using various extracts of the hardy kiwifruit species of Actinidia.

An extraction process used to produce a total water soluble extract and an ethyl acetate extract of hardy kiwifruit is described in U.S. Patent Publication No. 2004/0037909 and in Examples 1 and 2 of the present application. In additional embodiments of the present invention, the present inventors have now discovered that preparations of hardy kiwifruit produced by alternate methods of extraction, concentration or processing also produce compositions with immune regulatory activity, and particularly, the ability to suppress cytokine production in response to an antigen (e.g., an allergen). Such alternate preparations include, but are not limited to, fruit juice concentrate, fresh fruit concentrate, and boiled fresh fruit preparations.

The present inventors also demonstrate herein that extracts or other preparations of parts of the hardy kiwifruit plant other than the fruit itself have equivalent or superior immune regulatory activity as compared to the water soluble or ethyl acetate extracts of the fruit. For example, the present inventors have demonstrated the efficacy of extracts of the stem, root and bark of the hardy kiwifruit plant in suppressing cytokine production by antigen-stimulated splenocytes from allergen-sensitized mice.

The present inventors have also made the surprising discovery that preparations of hardy kiwifruit as described herein may serve as an effective adjunct to other therapies for various atopic conditions. For example, the present inventors have discovered that administration of a powdered form of the water soluble extract of A. arguta as described herein to adult human patients suffering from atopic dermatitis of moderate severity, significantly reduced the physician's global rating of clinical signs. Therefore, the present invention, in one embodiment, relates to the use of the hardy kiwifruit preparations described herein in combination with (or as an adjunct to) other therapeutic agents to treat atopic disease or other diseases associated with immune dysregulation. The present inventors believe that the compositions of the present invention can be used to enhance the efficacy of other therapeutic and nutritional therapies, particularly in patients with atopic conditions. This embodiment of the invention is discussed in detail below.

In yet another embodiment of the invention, the present inventors report herein that surprisingly, the process of drying hardy kiwifruit is a previously unappreciated, but important element to enhancing the bioactivity of the hardy kiwifruit. Moreover, the present inventors show herein that the dried hardy kiwifruit, extracted solely with room temperature water, exhibits similar bioactivity to that previously ascribed to hot water extracts or organic solvent extracts of hardy kiwifruit. Dried hardy kiwifruit extracted in cool or cold water is also encompassed, thus extending extraction to any temperature between 0° C. and 80° C. The present inventors also set forth herein that dried hardy kiwifruit that has not been extracted (e.g., dried slices of hardy kiwifruit) can have the bioactivity that was previously ascribed to extracts of hardy kiwifruit. Therefore, the present invention relates to any form of dried hardy kiwifruit, including extracts produced from previously dried hardy kiwifruit, as an agent or for the preparation of a composition for regulating the immune response in a mammal. More specific uses for this agent or composition are described below.

In another embodiment, the invention further relates to the use of any members of the family, Actinidiaceae, and particularly any members of the genus Actinidia, including, but not limited to, the common kiwi known as A. chinensis or A. deliciosa to provide compositions of kiwifruit with immune regulatory activity. In one embodiment, without being bound by theory, the present inventors believe that the process of drying other members of Actinidia can provide a composition of dried Actinidia having at least some of the biological activities that have been recognized for the hardy kiwifruit described herein. In another embodiment, the present inventors believe that other preparations of common kiwifruit (e.g., A. chinensis or A. deliciosa), including, but not limited to, preparations of any part of the fruit, the whole fruit, the stem, the leaf, the bark, or the root, and including any preparation or extract thereof, including dried preparations, non-extracted but processed preparations, fresh fruit, fruit juice, or any extract, concentrate, or fraction thereof, can have at least some of the biological properties that have been recognized for the hardy kiwifruit described herein.

A. arguta, A. polygama, and A. kolomikta belonging to Actinidiaceae, are naturally distributed in Siberia, the northern area of China, North and South Korea. More than 30 species belonging to Actinidiaceae has been reported. Among those, the fruit of A. chinensis or A. deliciosa have been named “kiwi” and are popular edible fruits. A. arguta and other fruit of the same genus (e.g., A. polygama, and A. kolomikta) have been used as materials of Chinese medicine named as ‘mihudo’ to treat liver disease, gastrointestinal disease and urogenital lithiasis without toxicity (Seoul National University Natural Products Science, Tradi-Medi Data Base, dongbang media Co. Ltd. 1999). However, prior to the invention described in U.S. Patent Publication No. 2004/0037909, there have been no reports or suggestions about the treatment and prevention of allergic disease and non-allergic inflammatory disease using by Actinidia fruit. Moreover, prior to the present invention, there have been no reports regarding the efficacy of preparations of the hardy kiwifruit other than the extracts described in U.S. Patent Publication No. 2004/0037909, regarding the efficacy of extracts prepared from parts of the hardy kiwifruit other than the fruit (berries), regarding the importance of various steps in the preparation process, such as drying, or of the effect of the hardy kiwifruit compositions on the efficacy of established treatments for atopic disease or other conditions associated with dysregulation of the mammalian immune system.

According to the present invention, reference to “hardy kiwifruit” refers to any of A. arguta, A. polygama, and A. kolomikta, or another species of the Actinidia genus related thereto that has the bioactive properties of A. arguta, A. polygama, and A. kolomikta as described herein, particularly with regard to the anti-allergy properties and the immune response/cytokine/leukotriene-regulatory properties demonstrated herein (e.g., see Examples).

In the preparation of any composition or preparation of the invention, including any extract described herein, one may use any one or more parts of the hardy kiwifruit or other species of Actinidia, including, but not limited to, the fruit (also referred to as the “berries” or the “kiwiberries”), the leaf, the stem, the bark and the root thereof.

General reference herein to an extract refers to a concentrated preparation of a substance (e.g., hardy kiwifruit) which is typically obtained by removing the active or desired constituents therefrom with a suitable solvent, and then evaporating some, all or nearly all the solvent and adjusting the residual mass or powder to a prescribed standard. The term “concentrate” refers to a form of substance which has had the majority of its base component, or solvent, removed. Accordingly, it will be apparent that the term “extract” as disclosed herein may be, in some embodiments, used interchangeably with the term “concentrate”. Reference herein to a crude extract refers to, in one embodiment, an extract of hardy kiwifruit that is obtained by extracting a preparation of a hardy kiwifruit with water, lower alcohols (e.g., methanol, ethanol and the like), or the mixtures thereof, and preferably distilled water or 50-90% ethanol, and more preferably 70% ethanol. A non-polar solvent soluble extract therefrom can be obtained by further extracting the soluble extract with a non-polar solvent such as hexane, ethyl acetate or dichloromethane solvent. Specific procedures for production of a crude extract and evaluation of the same are described in U.S. Patent Publication No. 2004/0037909, supra, and all such procedures are incorporated herein by reference. Additional bioassays for tracking, evaluating or confirming the preferred biological activities of a hardy kiwifruit composition according to the present invention are set forth in the Examples herein, and include both in vitro and in vivo assays.

According to the present invention, reference to “PG102T” refers generally to a total water-soluble extract (which may also be referred to herein as a concentrate) from a hardy kiwifruit described herein (e.g., A. arguta), prepared essentially as described in U.S. Patent Publication No. 2004/0037909, supra (e.g., see Example 1 of that publication), or as described in Example 1 herein. In a preferred embodiment, the total water-soluble extract is prepared from A. arguta, although it will be apparent to those of skill in the art that equivalent total water-soluble extracts can be prepared from other hardy kiwifruit, including, but not limited to, A. polygama and A. kolomikta. When the total water-soluble extract is produced by a large-scale process but using substantially the same basic steps as for the preparation of PG102T, as described in Example 3, the resulting preparation can be referred to herein as FD001. Reference to “PG102E” herein refers to the ethyl acetate fraction resulting from successive solvent partition of a PG102T preparation described above with chloroform, ethyl acetate, and n-butanol using extraction methods conventionally known in the art. Specific methods for producing an extract that is a PG102T extract and an extract that is a PG102E extract are described in Example 1. In another embodiment of the invention, a different ethyl acetate extract is produced by directly extracting FD001 (or PG102T) with ethyl acetate (i.e., there is no chloroform extraction prior to the ethyl acetate fraction). Again, in a preferred embodiment, an ethyl acetate extract is prepared from A. arguta, although it will be apparent to those of skill in the art that equivalent extracts can be prepared from other hardy kiwifruit, including, but not limited to, A. polygama and A. kolomikta.

In some embodiments of the invention, particularly when the fruit is dried as a step in the processing or preparation of an extract or concentrate or other preparation, extracts or concentrates or other preparations of the invention can be produced from any species of Actinidia. Accordingly, it is an object of the present invention to provide a composition, including a pharmaceutical composition, a cosmetic composition, or a composition useful as or with a health food product, health food or beverage, or food additive (including human and animal (including domestic pet) food additives), that comprises such extracts of hardy kiwifruit or other species of Actinidia, if desired. Such compositions are intended for use in any method of the invention, including to selectively regulate Th1 and Th2 immune responses in a patient (i.e., in a mammal), such as the method for preventing or treating allergic and non-allergic inflammatory disease or providing an anti-viral or anti-cancer pharmaceutical or nutraceutical by administration of such compositions. Other additives and components of the compositions, as well as the dosing and administration strategies described herein apply to this object of the invention as well.

In the preparation of any composition described herein, in addition to the extracts described herein, including the extracts/concentrates described specifically above, included in the invention is the use of whole fruits of hardy kiwifruit, or fruit preparations that are processed, but not extracted, including, but not limited to, fresh fruit, crushed fruit (dried or fresh), boiled fruit (dried or fresh), cooked fruit, dried fruit, pressed fruit, frozen fruit, and condensed fruit. Accordingly, it is an object of the present invention to provide a composition, including a pharmaceutical composition, a cosmetic composition, or a composition useful as or with a health food product, health food or beverage, or food additive, that comprises such whole fruits of hardy kiwifruit or fruit preparations that may be processed in some manner (e.g., dried, boiled, etc.), but that are not necessarily extracted. Therefore, in one embodiment, the present invention relates to preparations of hardy kiwifruit (and common kiwifruit) that has not been extracted. Any of such compositions described herein are intended for use in any method of the invention, including to selectively regulate Th1 and Th2 immune responses in a patient (i.e., in a mammal), such as the method for preventing or treating allergic and non-allergic inflammatory disease or providing an anti-viral or anti-cancer pharmaceutical or nutraceutical by administration of such compositions. Other additives and components of the compositions, as well as the dosing and administration strategies described herein apply to this object of the invention as well.

Furthermore, in the preparation of any composition described herein, in addition to the extracts described herein, included in the invention is the use of the juice of a hardy kiwifruit, produced from any part of the hardy kiwifruit and by any suitable process. The juice can be used as produced directly from the fruit (i.e., not diluted or concentrated), the juice can be diluted, or it can be concentrated to form a fruit juice concentrate. For example, as described in Example 3, fresh kiwifruit can be run through a conventional juicer. The juicer may remove the skins from the fruit resulting in a mixture of seeds, pulp, and juice. This mixture can then be treated (e.g., by centrifugation or pressing) to remove the juice from the solids and, if desired, this juice can be concentrated (e.g., by evaporation, distillation, ultrafiltration, etc.) to provide a concentrated fruit juice (i.e., fruit juice concentrate). Such compositions are intended for use in any method of the invention, and particularly in the method for preventing or treating allergic and non-allergic inflammatory disease or providing an anti-viral or anti-cancer pharmaceutical or nutraceutical by administration of such compositions. Other additives and components of the compositions, as well as the dosing and administration strategies described herein apply to this object of the invention as well.

Furthermore, in the preparation of any composition described herein, as an alternative to the extracts described in U.S. Patent Publication No. 2004/0037909, included in the invention is the use of other products of processing of the hardy kiwifruit, including, but not limited to, a room temperature water extract of fruit, including dried fruit; a water extract of hardy kiwifruit performed in water having a temperature of less than room temperature; a water extract or other extract of the root, leaf, stem, or bark; or a water extract or concentrate or other extract of any of the fruit, leaf, stem or bark, that is not dried prior to extraction (e.g., extracted fresh fruit). The present inventors propose herein that the hardy kiwifruit can be extracted in any temperature water ranging from 0° C. to 80° C., including room temperature and cooler (e.g., from about 0° C. to about 25° C.).

According to the present invention, general reference to a “dried hardy kiwifruit” or a “dried kiwifruit” includes any form of a hardy kiwifruit or other kiwifruit (e.g., common kiwi) that has been dried by any process. The term “kiwifruit” can be used herein to generically refer to any member of the genus Actinidia, and includes the members of the hardy kiwifruit as discussed above, as well as members of the common kiwi, also as discussed above. Therefore, a dried kiwifruit includes any dried part of the kiwifruit (fruit, leaf, stem, root, etc.), and includes dried whole fruits, dried sliced fruit, dried crushed fruit, dried diced fruit, and dried condensed fruit, as well as any extracts of kiwifruit, wherein the material that is extracted is first dried prior to extraction. The extracts themselves need not be dried or processed further, although that is generally the most useful form for the extracts for formulation into compositions and for storage. Preferred methods of further concentration of the extracts for further use include, but are not limited to, evaporation, distillation, ultrafiltration, reverse osmosis, precipitation, adsorption to and elution from a stationary phase, and extraction into alternative solvents. Preferred methods of drying the extracts or concentrates for further use include, but are not limited to, tray drying, spray drying and freeze drying, both with or without the use of drying aids or excipients such as maltodextrin, microcrystalline cellulose and starch. In one embodiment, a preferred dried kiwifruit for use in the present invention is a dried kiwifruit preparation that is not subsequently extracted.

Therefore, the compositions and methods described herein apply to the use of any hardy kiwifruit, including the use of any part of the fruit, stem, leaf, bark or root of the hardy kiwifruit, or any extract or concentrate or fraction thereof, any form of the whole fruit or processed but not extracted fruit, fruit juice, or any extract or concentrate or fraction thereof, and further including hardy kiwifruit plant parts (the plant parts including the fruit, stem, leaf, bark, and/or root) and plant part preparations that are prepared using a process that includes a step of drying.

It is therefore an object of the present invention to provide a composition, including a pharmaceutical composition, a nutraceutical composition, a food additive, a health food (including a beverage or a food material), or a cosmetic composition, comprising, consisting essentially of, or consisting of, the hardy kiwifruit described herein (i.e., including any part of the fruit, the whole fruit, the stem, the leaf, the bark, or the root, and including any preparation or extract or concentrate thereof, including dried preparations, non-extracted but processed preparations, fresh fruit, fruit juice, or any extract, concentrate or fraction thereof). In one embodiment, the present invention provides a crude extract, a total water-soluble extract, or an ethyl acetate extract of the hardy kiwifruit. In all cases, the hardy kiwifruit preparation is an active ingredient for use to selectively regulate Th1 and Th2 immune responses in a patient (i.e., in a mammal). In particular, the composition has a biological activity selected from at least one of the following activities: (a) reduces the number of IgE-producing B cells in a patient; (b) reduces the amount of IgE produced in a patient (e.g., in the serum or plasma); (c) decreases production and/or levels of at least one Th2 cytokine (e.g., IL-4, IL-5, IL-10); (d) increases the level of at least one Th1 cytokine (e.g., IL-12, IFN-γ); (e) decreases the level of expression of the transcription factor, GATA-3; (f) increases the level of expression of the transcription factor T-bet; (g) increases the level of expression of the transcription factor NFATc2; (h) increases the number of IgG2a-producing B cells in a patient; (i) increases the amount of IgG2a produced in a patient; (j) enhanced production or activity of Th1 T lymphocytes (e.g., CD4+, IFN-γ+), particularly at a site of inflammation; (k) decreases production or activity of Th2 T lymphocytes (e.g., CD4+, IL-4+), particularly at a site of inflammation; (l) reduces the number of IgG1-producing B cells in a patient; (m) reduces the amount of IgG1 produced in a patient; and/or (n) reduces the level of or production of at least one leukotriene in the patient.

The compositions described above can be used for the prevention and/or treatment of any disease or condition in which regulation of the immune response in the manner described herein would be, or could be predicted to be, beneficial to a patient.

As used herein, the phrase “protected from a disease” refers to reducing the symptoms of the disease; reducing the occurrence of the disease, and/or reducing the severity of the disease. Protecting a patient can refer to the ability of a composition of the present invention, when administered to a patient, to prevent a disease from occurring and/or to cure or to alleviate at least one, and preferably more than one, disease symptoms, signs or causes. As such, to protect a patient from a disease includes both preventing disease occurrence (prophylactic treatment) and treating a patient that has a disease (therapeutic treatment) to reduce the symptoms of the disease. In particular, protecting a patient from a disease or enhancing another therapy is accomplished by regulating a given activity such that a beneficial effect is obtained. A beneficial effect can easily be assessed by one of ordinary skill in the art and/or by a trained clinician who is treating the patient. The term, “disease” refers to any deviation from the normal health of a mammal and includes a state when disease symptoms are present, as well as conditions in which a deviation (e.g., infection, gene mutation, genetic defect, etc.) has occurred, but symptoms are not yet manifested.

In general, the biological activity or biological action of an agent described herein, including a hardy kiwifruit composition, extract of hardy kiwifruit, or any other preparation thereof, refers to any function(s) exhibited or performed by the agent that is ascribed to the naturally occurring form of the agent as measured or observed in vivo (i.e., in a natural physiological environment wherein the agent is used) or in vitro (i.e., under laboratory conditions). Modifications of an agent, such as by changing the processing or preparation of the agent or purification of the agent, may result in agents having the same biological activity as the naturally occurring agent, or in agents having decreased or increased biological activity as compared to the naturally occurring agent.

Accordingly, it is an object of the present invention to provide a composition, including a pharmaceutical composition or a nutraceutical (nutritional) composition, comprising the hardy kiwifruit described herein (i.e., including any part of the fruit, the whole fruit, the stem, the leaf, the bark, or the root, and including any preparation or extract or concentrate thereof, including dried preparations, non-extracted but processed preparations, fresh fruit, fruit juice, or any extract or concentrate or fraction thereof), and in one embodiment, a crude extract, a total water-soluble extract, or an ethyl acetate extract of the hardy kiwifruit (which can collectively be referred to as active ingredients of the invention or as hardy kiwifruit preparations of the invention), as an active ingredient for regulating an immune response in the mammal, and more particularly for regulating a Th2 and/or a Th1 immune response in a mammal, and even more particularly, for enhancing a Th1 response in a mammal and/or suppressing a Th2 response in a mammal. Such active ingredients are useful for the treatment and/or prevention of a variety of conditions and diseases, including, but not limited to, allergic disease, non-allergic inflammatory disease, viral infection and cancer. The composition can further comprise or be used in conjunction with additional therapeutic or nutraceutical agents for the prevention, treatment, and/or improvement of any of the above-described conditions or diseases. According to the present invention, nutritional applications include any applications of the invention directed to the provision of nutrients and nutritional agents to maintain, stabilize, enhance, strengthen, or improve the health of an individual or the organic process by which an organism assimilates and uses food and liquids for functioning, growth and maintenance, and which includes nutraceutical applications. Therapeutic applications include any applications of the invention directed to prevention, treatment, management, healing, alleviation and/or cure of a disease or condition that is a deviation from the health of an individual. Other applications of the invention include, for example, cosmetic applications.

It is also an object of the present invention to provide a hardy kiwifruit described herein (i.e. a hardy kiwifruit preparation, including any part of the fruit, the whole fruit, the stem, the leaf, the bark, or the root, and including any preparation or extract or concentrate thereof, including dried preparations, non-extracted but processed preparations, fresh fruit, fruit juice, or any extract or concentrate or fraction thereof), and in one embodiment, a crude extract, a total water-soluble extract, or an ethyl acetate extract of the hardy kiwifruit, for the preparation of a therapeutic or nutraceutical agent for regulating an immune response in the mammal, and more particularly for regulating a Th2 and/or a Th1 immune response in a mammal, and even more particularly, for enhancing a Th1 response in a mammal and/or suppressing a Th2 response in a mammal. Such active ingredients are useful for the treatment and/or prevention of a variety of conditions and diseases, including, but not limited to, allergic disease, non-allergic inflammatory disease, viral infection and cancer. The agent can be used in conjunction with additional therapeutic or nutraceutical agents for the prevention, treatment, and/or improvement of any of the above-described conditions or diseases.

It is another object of the present invention to provide a health food or food additives comprising the hardy kiwifruit described herein (i.e. a hardy kiwifruit preparation, including any part of the fruit, the whole fruit, the stem, the leaf, the bark, or the root, and including any preparation or extract or concentrate thereof, including dried preparations, non-extracted but processed preparations, fresh fruit, fruit juice, or any extract or concentrate or fraction thereof), and in one embodiment, a crude extract, a total water-soluble extract, or an ethyl acetate extract of the hardy kiwifruit, together with any acceptable additive for regulating an immune response in the mammal, and more particularly for regulating a Th2 and/or a Th1 immune response in a mammal, and even more particularly, for enhancing a Th1 response in a mammal and/or suppressing a Th2 response in a mammal. Such health food or health food additives are useful for the treatment and/or prevention of a variety of conditions and diseases, including, but not limited to, allergic disease, non-allergic inflammatory disease, viral infection and cancer.

It is still another object of the present invention to provide an animal feed or feed additive comprising the hardy kiwifruit described herein (i.e., a hardy kiwifruit preparation, including any part of the fruit, the whole fruit, the stem, the leaf, the bark, or the root, and including any preparation or extract or concentrate thereof, including dried preparations, non-extracted but processed preparations, fresh fruit, fruit juice, or any extract or concentrate or fraction thereof), and in one embodiment, a crude extract, a total water-soluble extract, or an ethyl acetate extract of the hardy kiwifruit, as an essential component for regulating an immune response in the mammal, and more particularly for regulating a Th2 and/or a Th1 immune response in a mammal, and even more particularly, for enhancing a Th1 response in a mammal and/or suppressing a Th2 response in a mammal. Such animal feeds or animal feed additives are useful for the treatment and/or prevention of a variety of conditions and diseases, including, but not limited to, allergic disease, non-allergic inflammatory disease, viral infection and cancer.

It is still another object of the present invention to provide a topical or cosmetic composition comprising the hardy kiwifruit described herein (i.e. a hardy kiwifruit preparation, including any part of the fruit, the whole fruit, the stem, the leaf, the bark, or the root, and including any preparation or extract or concentrate thereof, including dried preparations, non-extracted but processed preparations, fresh fruit, fruit juice, or any extract or concentrate or fraction thereof), and in one embodiment, a crude extract, a total water-soluble extract, or an ethyl acetate extract of the hardy kiwifruit, for regulating an immune response in the mammal, and more particularly for regulating a Th2 and/or a Th1 immune response in a mammal, and even more particularly, for enhancing a Th1 response in a mammal and/or suppressing a Th2 response in a mammal. Such cosmetic compositions are useful for the treatment and/or prevention of a variety of conditions and diseases, including, but not limited to, allergic disease (including allergic diseases of or affecting the skin), non-allergic inflammatory disease, viral infection and cancer.

Any of the compositions, additives, or agents described herein may additionally comprise at least one conventional carrier, adjuvant or diluent. For example, the composition according to the present invention can include pharmaceutically acceptable carriers, adjuvants or diluents, e.g., lactose, dextrose, sucrose, sorbitol, mannitol, xylitol, erythritol, maltitol, starches, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil. The formulations may additionally include fillers, anti-agglutinating agents, lubricating agents, wetting agents, flavoring agents, emulsifiers, preservatives and the like. The compositions of the invention may be formulated so as to provide quick, sustained or delayed release of the active ingredient after their administration to a patient.

For example, the compositions of the present invention can be dissolved in oils, propylene glycol or other solvents that are commonly used to produce an injection. Suitable examples of the carriers include physiological saline, polyethylene glycol, ethanol, vegetable oils, isopropyl myristate, etc., but are not limited to these carriers. For topical administration, the compounds of the present invention can be formulated in the form of ointments and creams.

Compositions or formulations of the present invention may be prepared in any form, such as oral dosage form (effervescent tablet, effervescent powder, powder, tablet, capsule, soft capsule, aqueous medicine, syrup, elixirs pill, powder, sachet, granule), or topical preparation (cream, ointment, lotion, gel, balm, patch, paste, spray solution, aerosol and the like), or injectable preparation (solution, suspension, emulsion).

The composition of the present invention in pharmaceutical dosage forms may be used alone or in appropriate association or combination with other pharmaceutically active compounds, including anti-inflammatory compounds, anti-allergy compounds, or any other compounds or compositions that can regulate an immune response or provide a benefit to a patient. Compounds that are particularly desirable for use in the compositions and formulations of the present invention are described in detail herein.

The composition of the present invention can also be provided as a health food that includes the hardy kiwifruit preparations of the invention (e.g., various foods, beverage, gum, vitamin complex, health improving food and the like). The health food can be provided as a food item, powder, granule, tablet, chewing tablet, capsule or beverage etc. Child or infant foods are also included in the compositions of the invention, such as modified milk powder, infant formulas, and modified infant or children's food.

Suitable food products into which a composition or agent of the invention can be introduced to produce a health food product include, but are not limited to, fine bakery wares, bread and rolls, breakfast cereals, processed and unprocessed cheese, condiments (ketchup, mayonnaise, etc.), dairy products (milk, yoghurt), puddings and gelatin desserts, carbonated drinks, teas, powdered beverage mixes, processed fish products, fruit-based drinks (including fruit juices), vegetable-based drinks (including vegetable juices), chewing gum, hard confectionery, frozen dairy products, processed meat products, nut and nut-based spreads, pasta, processed poultry products, gravies and sauces, potato chips and other chips or crisps, chocolate and other confectionery (cookies, candy, licorice), ice creams, dehydrated foods, cut or processed food products (e.g., fruits, vegetables), spices, alcoholic beverages, noodles, fermented foods, soups and soup mixes, soya based products (milks, drinks, creams, whiteners), vegetable oil-based spreads, and vegetable-based drinks. A composition of the present invention can also be used with a food, such as placed onto, poured onto or mixed into the food at the time of serving.

Compositions described above, and particularly cosmetic formulations containing the above-identified compositions, may be prepared in any form such as skin, lotion, cream, essence, toner, emulsion, pack, soap, shampoo, rinse, cleanser, body washing solution, washing solution, treatment, gel, balm, spray solution and the like.

Any of the above compositions of the present invention can further include one or more of lactose, casein, dextrose, glucose, sucrose and sorbitol.

Any of the compositions, preparations, additives, or agents described herein may additionally comprise at least one active agent (i.e., active compound, active component). The additional active agent can be a pharmacologically active agent and/or a nutritionally active agent. Active agents typically contribute at least one additional desirable, nutritional, and/or therapeutic and/or pharmacological property to a composition, in addition to the kiwifruit preparation described herein. Active agents can be included in a composition, preparation, additive, or other formulation of the invention in any effective amount. An effective amount is an amount sufficient to achieve the desired effect imparted by the agent, such as an effect on the health or nutrition of a subject (e.g., a therapeutic or nutritional effect), a taste effect, an aroma effect, a visual effect, etc. One of skill in the art will be able to determine the appropriate amount of additional agents to add to a composition of the invention.

For example, any of the compositions provided by or useful in the present invention can include one or more natural products as an active agent, including, but not limited to, fatty acids and polyketides; organic acids and miscellaneous small organic compounds; aromatic amino acids and phenylpropanoids; terpenoids; alkaloids; corrins and porphyrins; linear and cyclic peptides, depsipeptides, and other amino acids derivatives; nucleosides and nucleotides; carbohydrates; proteins, cells and cell fragments; herbal preparations and spices; minerals; sterilizers; seasonings; vitamins; electrolytes; and other natural agents.

Other components (compounds or agents) that may be added to a composition of the invention include synthetic flavoring agents, a coloring agent, a processing agent, an alginic acid or the salt thereof, an organic acid, a protective colloidal adhesive, a pH controlling agent, stabilizer, a preservative, a glycerin, an alcohol, a carbonation agent, or any other essential agent of a formulation (for nutritional or therapeutic use by any method of administration), a food, or a beverage.

Particularly preferred components (active agents) to combine with a hardy kiwifruit preparation of the invention or add to a composition containing such preparation include, but are not limited to: probiotics; bacterial cell walls and fragments; whey protein; taurine; alanine; fatty acids (e.g., conjugated linolenic acid, eicosapentaenoic acid, docosahexaenoic acid, γ-linolenic acid, α-linolenic acid, dihomo-γ-linolenic acid, stearidonic acid); mono-, di-, and triglycerides (composed of any combination of the fatty acids described above); inositol; turmeric; curcumin; rosemary; rosemarinic acid; methylsulfonylmethane (MSM); ginseng; ginger; proanthocyanidin; β-carotene; and any other preparation of a different species of kiwifruit than that used as the primary bioactive component, including any member of Actinidiaceae, and particularly any members of the genus Actinidia, including common kiwi species (e.g., A. chinensis or A. deliciosa) and hardy kiwifruit species (e.g., A. arguta, A. polygama, and A. kolomikta).

Fatty acids and polyketides include, but are not limited to: saturated fatty acids (e.g., α-lipoic acid (R, S, or R,S); unsaturated fatty acids (e.g., conjugated linolenic acid, eicosapentaenoic acid, docosahexaenoic acid, γ-linolenic acid, α-linolenic acid, dihomo-γ-linolenic acid, stearidonic acid); fatty acid esters; monoglycerides, diglycerides, and triglycerides (composed of any combination of the fatty acids described above); acetylenic fatty acids; branched-chain fatty acids; prostaglandins; thromboxanes; leukotrienes; aromatic polyketides; macrolides and polyethers; lipid extracts (e.g., marine oils, Echium oil, borage oil, olive oil); and lecithin.

Organic acids and miscellaneous small organic compounds include, but are not limited to, citric acid; fumaric acid; guaiacol; methylsulfonylmethane (MSM); and ascorbic acid.

Aromatic amino acids and phenylpropanoids include, but are not limited to, aromatic amino acids and benzoic acids (e.g., benzoic acid, gallic acid, gentisic acid, p-hydroxybenzoic acid, protocatechuic acid, vanillic acid, salicylic acid, syringic acid); cinnamic acids (e.g., hydroxytyrosol, curcumin, rosmarinic acid, ar-turmerone, caffeic acid, eugenol, chlorogenic acid, neochlorogenic acid, cinnamic acid, ferulic acid, o-coumaric acid, p-coumaric acid); lignans and lignin; phenylpropenes; coumarins; styrylpyrones; flavonoids (e.g., anthocyanidins, such as delphinidin; proanthocyanidins; catechins such as catechin, epicatechin, and theaflavin; flavonols, such as avicularin, hyperoside, quercitrin, isoquercitrin, kaempferol, myricetin, rutin; flavanones, such as naringenin; chalcones, such as phloretin; isoflavones, such as vitexin); stilbenes; flavonolignans; isoflavonoids; and terpenoid quinines (e.g., K vitamins and tocopherols (vitamin E) such as tocotrienols).

Terpenoids include, but are not limited to, monoterpenes (e.g., β-pinene, bomeol, carvacvol, geraniol, thymol, 1,8-cineol, terpineol); iridoids (e.g., monotropein); β-ionone (e.g., thirteen carbon precursor to A vitamins); sesquiterpenes (e.g., caryophyllene, farnesol); diterpenes (e.g., A vitamins); sesterterpenes; triterpenes (e.g., α-amyrin, lupeol, ursolic acid); tetraterpenes; and carotenoids (e.g., lycopene, β-carotene, lutein, astaxanthin, canthaxanthin).

Alkaloids include, but are not limited to, pyrrolidine alkaloids, tropane alkaloids, pyrrolizidine alkaloids, piperidine alkaloids, quinolizidine alkaloids, indolizidine alkaloids, pyridine alkaloids, phenylethylamines, tetrahydroisoquinoline alkaloids, galanthamines, indole alkaloids, β-carboline alkaloids, terpenoid indole alkaloids, quinoline alkaloids, pyrroloindole alkaloids, ergot alkaloids, quinazoline alkaloids, quinoline and acridine alkaloids, imizadole alkaloids, piperidine alkaloids, ephedrines, caps aisins, pyridine monoterpene alkaloids, aconitines, purine alkaloids (e.g., allantoin, caffeine, theophylline).

Corrins and porphyrins include, but are not limited to, B vitamins.

Linear and cyclic peptides, depsipeptides, and other amino acids derivatives include, but are not limited to, simple amino acids and their derivatives (e.g., L-acetyl carnitine, choline, taurine, alanine), linear peptides, cyclic peptides (e.g., cyclosporins), cyclic depsipeptides, β-lactams, cyanogenic glycosides, glucosinolates, cysteine sulphoxides.

Carbohydrates include, but are not limited to, monosaccharides (e.g., inositol), polysaccharides (e.g., fructo-oligosaccharides, such as inulin (any chain length); galacto-oligosaccharides; chitin and chitosan).

Other natural materials include, but are not limited to, proteins (e.g., whey protein and superoxide dismutase); cells and cell fragments (e.g., probiotics, meaning live, intact microorganisms such as, e.g., Lactobacillus spp., bacterial cells and cell wall fragments, fungal/yeast cells and cell wall fragments); herbal preparations and spices (e.g., ginseng, huang, turmeric, rosemary, ginger); minerals (e.g., K, Mg, Ca, Mn, Fe, Cu, Zn, B, Si, Se). Metabolites and derivatives of any of these compounds are also encompassed by the present invention.

In one embodiment of the invention, the composition of the invention is administered as an adjunct therapy for a conventional therapy for a condition or disease. Therefore, compositions of the invention may include one or more therapeutic agents (e.g., medicines), which can also be referred to herein as active agents, used to treat a condition or disease that can be treated or ameliorated by regulation of immune responses. Such therapeutic agents include, but are not limited to, antihistamines (any type, including systemic, topical, inhaled, and including H1 and H2 blockers), antibodies (e.g., anti-IgE, anti-IL-10), antibiotics, cyclosporins, antimycotics, respiratory function controllers, analgesics, β-agonists (long or short acting), leukotriene modifiers (inhibitors or receptor antagonists), cytokine or cytokine receptor antagonists, phosphodiesterase inhibitors, sodium cromoglycate, nedocrimil, theophylline, caffeine, carbobenzoxy beta-alanyl taurine, inhibitors of T cell function and other anti-inflammatory agents.

Any of the above compositions may additionally comprise one or more than one organic acid (e.g., citric acid, fumaric acid, adipic acid, lactic acid, malic acid, ascorbic acid), phosphate (e.g., phosphate, sodium phosphate, potassium phosphate, acid pyrophosphate, polyphosphate), and/or natural anti-oxidants (e.g., polyphenol, catechin, α-tocopherol, rosemary extract, vitamin C, green tea extract, licorice root extract, chitosan, tannic acid, phytic acid, etc).

The compositions of the present invention, and particularly cosmetic compositions or compositions that are formulated for topical administration, including therapeutic compositions (but not limited to cosmetic or other topical compositions) can comprise additional additives including, but not limited to, water soluble vitamin, lipid soluble vitamin, peptide polymer, polysaccharide polymer, sphingolipid, glycosaminoglycans, B-glucan and sea-weed extract. In addition, one may add the compositions and agents of the present invention to existing cosmetics and washing solutions. Such compositions can be used as creams, lotions, massage packs or oils, and body washing solutions, soap, shampoos and the like.

Preferred water soluble vitamins are any one which can be mixed with cosmetics or other topical formulations, however, various vitamin such as vitamin B1, B2, B6, pyridoxine, pyridoxine HCl, vitamin B12, pantothenic acid, nicotinic acid, nicotinamide, folic acid, vitamin C, vitamin H etc, their salt thereof such as thiamin HCl salt, ascorbic acid Na salt etc or their derivatives thereof such as ascorbic acid-2-phosphonic acid Na salt, ascorbic acid-2-phosphonic acid Mg salt are preferable, and those can be obtained by conventional methods such as microbial conversion methods, purification methods from the microbial cultivates, enzymatic methods or chemical synthetic methods.

Preferred lipid soluble vitamins are any one which can be mixed with cosmetics or other topical formulations, however, various vitamin such as vitamin A, D2, D3, E (d1-α-tocopherol, d-α-tocopherol, d-δ-tocopherol) and their derivatives such as palmitic acid ascorbate, stearic acid ascorbate, dipalmitic acid ascorbate, acetic acid-dl-α-tocopherol, nicotinic acid dl-α-tocopherol vitamin E, dl-pantothenyl alcohol, d-pantothenyl alcohol, pantothenyl ethylether etc. containing the lipid soluble vitamin used in examples of present invention are preferable and those can be obtained by conventional methods such as microbial conversion methods, purification methods from the microbial cultivates, enzymatic methods or chemical synthetic methods.

Preferred peptide polymers are any one which can be mixed with cosmetics or other topical formulations; however, collagen, hydrolysable collagen, gelatin, elastin, hydrolys able gelatin, or keratin, etc., containing the peptide polymer used in examples of present invention are preferable.

Preferred polysaccharide polymers are any one which can be mixed with cosmetics or other topical formulations, however, hydroxy ethyl cellulose, xanthin gum, hyaluronic acid Na, chondroitin sulfate or their salt (Na salt etc) and the like are preferable. For example, chondroitin sulfate or the salt thereof etc. can be used by being purified from mammals or fish ordinarily.

Preferred sphingolipids are any one which can be mixed with cosmetics or other topical formulations, however, squalane, ceramide, pit-sphingosin, sphingo-lipopolysaccharide and the like are preferable. Sphingo-lipids can be obtained by purification from mammals, fish, shellfish, yeast, or plants etc. using conventional methods.

Preferred seaweed extracts are any one which can be mixed with cosmetics or other topical formulations, however, the extract of brown algae, red algae, green algae and the like, or the purified carrageenan, alginic acid, arginic acid, Na, K, or glycosaminoglycans isolated therefrom are preferable. Algal extracts can be obtained by purification from seaweed using conventional methods.

The cosmetic and other topical compositions of the present invention may be combined with other ingredients or combined with a conventional cosmetic or topical composition, if necessary, together with above described hardy kiwifruit preparations. Such other ingredients include, but are not limited to, oil ingredients, humectants, emollients, surface active agents, organic or inorganic dye, organic powder, ultraviolet ray absorbing agent, preservatives, antiseptics, antioxidants, plant extract, pH controller, alcohol, pigments, perfumes, refrigerants, antihidrotic, distilled water etc. Preferable oil ingredients may comprise ester oil, hydrocarbon oil, silicone oil, fluoride oil, animal oil, plant oil and so on.

Preferred ester oils include, but are not limited to, glyceryl tri-2-ethyl hexanoic acid, cetyl 2-ethyl hexanoic acid, isopropyl myristic acid, butyl myristic acid, isopropyl palmitic acid, ethyl stearic acid, octyl palmitic acid, isocetyl isostearic acid, butyl stearic acid, ethyl linoleic acid, isopropyl linoleic acid, ethyl oleic acid, isocetyl myristic acid, isostearyl myristic acid, isostearyl palmitic acid, octyldodecyl myristic acid, isocetyl isostearic acid, diethyl sebasic acid, isopropyl adipic acid, isoalkyl neopetanoic acid, glyceryl tri(capryl, capric acid), trimethylopropane tri-2-ethyl hexanoic acid, trimethylopropane triisostearic acid, pentaerythritol tetra-2 ethyl hexanoic acid, cetyl caprylic acid, decyl lauric acid, hexyl lauric acid, decyl myristic acid, myristyl myristic acid, cetyl myristic acid, stearyl stearic acid, decyl oleic acid, cetyl licinoleic acid, isostearyl lauric acid, isotridecyl myristic acid, isocetyl palmitic acid, octyl stearic acid, isocetyl stearic acid, isodecyl oleic acid, octyldodecyl oleic acid, octyldodecyl linoleic acid, isopropyl isostearic acid, cetostearyl 2-ethyl hexanoic acid, stearyl 2-ethyl hexanoic acid, hexyl isostearic acid, ethylene glycol dioctanoic acid, ethylene glycol dioleic acid, propylene glycol dicapric acid, propylene glycol di(capryl, capric acid), propylene glycol dicaprylic acid, neopentylglycol dicapric acid, neopentylglycol dioctanoic acid, glyceryl tricaprylic acid, glyceryl triundecylic acid, glyceryl triisopalmitic acid, glyceryl triisostearic acid, octyldodecyl neopentanoic acid, isostearyl octanoic acid, octyl isononanoic acid, hexyldecyl neodecanoic acid, octyldodecyl neodecanoic acid, isocetyl isostearic acid, isostearyl isostearic acid, octyldecyl isostearic acid, polyglycerin oleanoic acid ester, polyglycerin isostearic acid ester, triisocetyl citric acid, triisoalkyl citric acid, triisooctyl citric acid, lauryl lactic acid, myristyl lactic acid, cetyl lactic acid, octyldecyl lactic acid, triethyl citric acid, acetyltriethyl citric acid, acetyl tributyl citric acid, trioctyl citric acid, diisostearyl maleic acid, di 2-ethylhexyl hydroxy stearic acid, 2-ethyl hexyl succinic acid, diisobutyl adipic acid, diisopropyl sebasinic acid, dioctyl sebacinic acid, cholesteryl stearic acid, cholesteryl isostearic acid, cholesteryl hydroxy stearic acid, cholesteryl hydroxy stearic acid, cholesteryl oleic acid, dihydrocholesteryl oleic acid, pitsteryl isostearic acid, pitsteryl oleic acid, isocetyl 12-stealoyl hydroxy stearic acid, stearyl 12-stealoyl hydroxy stearic acid, isostearyl 12-stealoyl hydroxy stearic acid.

Preferred hydrocarbon oils described above may comprise liquid paraffin, α-olefin oligomer, isoparaffin, ceresin, paraffin, liquid isoparaffin, polybuden, microcrystalline wax, vaselin and the like.

Preferred silicone oils may comprise polymethylsilicone, methylphenylsilicone, methylcyclopolysiloxane, octamethylpolysiloxane, decamethylpolysiloxane, dodecamethylcyclosiloxane, dimethyl siloxane-methyl cetyloxysiloxane copolymer, dimethyl siloxane-methyl stealloxysiloxane copolymer, alkyl modified silicone oil, amino modified silicone oil and the like.

Preferred fluoride oil can comprise perfluoropolyether and the like.

Preferred animal or plant oils can comprise avocado oil, almond oil, olive oil, sesame oil, rice husk oil, safflower oil, soybean oil, corn oil, rapeseed oil, canola oil, palm kernel oil, palm oil, sunflower oil, cotton seed oil, coconut palm oil, cucui nut oil, wheat embryo bud oil, rice embryo bud oil, shea butter, evening-primrose oil, macadamia nut oil, menhaden oil and other fish body oils, egg yolk oil, lanolin, hempseed oil, mink oil, orange roughy oil, jojoba oil, carnauba wax, liquid lanolin and the like.

Preferred humectants can comprise water-soluble low molecular humectants, lipophilic low molecular humectants, water-soluble polymer and lipid soluble polymer.

Specifically, preferable water soluble low molecular humectants can comprise cerin, glutamine, sorbitol, mannitol, pyrrolidone-carboxylic acid Na, glycerin, propylene glycol, 1,3-butylene glycol, ethylene glycol, polyethylene glycol (polymerization index. >2), polypropylene glycol (polymerization index >2), lactic acid, lactate salt and the like.

Preferred lipid soluble low molecular humectants can comprise cholesterol, cholesteryl ester and the like.

Preferred water-soluble polymers can comprise carboxy vinyl polymer, poly asparaginic acid salt, tragacanth, xanthin gum, HMC (hydroxy methyl celluose), HEC (hydroxy ethyl celluose), HPC (hydroxy propyl celluose), carboxymethylcellulose, water-soluble chitin, chitosan, dextrin and the like.

Preferred lipid soluble polymers can comprise polyvinylpyrrolidone-eicocene copolymer, polyvinylpyrrolidone-hexadecene copolymer, nitrocellulose, dextrin fatty acid ester, silicone polymer and the like.

Preferred emollients can comprise long chain acyl glutamic acid cholesteryl ester, cholesteryl hydroxy stearic acid, 12-hydroxy stearic acid, rogic acid, lanolin fatty acid cholesteryl ester and the like.

Preferred surface-active agents can comprise nonionic surfactants, anionic surfactants, cationic surfactants, amphivalent surfactants and the like.

Specifically, preferred non-ionic surfactants can comprise self-emulsified monostearic acid glycerin, propylene glycol fatty acid ester, glycerin fatty acid ester, polyglycerin fatty acid ester, sorbitan fatty acid ester, polyoxyethylene (POE) sorbitan fatty acid ester, POE sorbitan fatty acid ester, POE glycerin fatty acid ester, POE alkyl ether, POE fatty acid ester, POE solid pimaja oil, POE pimaja oil, POE-POP copolymer, POE-POP alkyl ether, polyether modified silicone, lauric acid alkanol amide, alkyl amine oxide, hydrogen addition soybean phospholipid and the like.

Preferred anionic surfactants can comprise fatty acid soap, α-acyl sulfonic acid salt, alkyl sulfonic acid salt, alkyl ally sulfonic acid, alkyl naphthalene sulfonic acid salt, alkyl sulfonic acid salt, POE alkylether sulfate salt, alkyl amide sulfate salt, alkyl phosphate salt, POE alkyl phosphate salt, alkylamide phosphate salt, alkyloylalkyl taurine salt, N-acyl-amino acid salt, POE alkyl ether carboxylic acid salt, alkyl sulfo succinic aid salt, alkyl sulfo-acetic acid salt, acylated hydrolysable collagen peptide salt, perfluoro alkyl phosphate ester and the like.

Preferred cationic surfactants can comprise alkyl trimethyl ammonium chloride, stearyl trimethyl ammonium chloride, stearyl trimethyl ammonium bromide, setostearyltrimethyl ammonium chloride, distearyl dimethyl ammonium chloride, stearyl dimethyl benzyl ammonium chloride, phenyl trimethyl ammonium bromide, benzalkonium chloride, diethylamino ethyl amide stearic acid, dimethylaminopropyl amide stearic acid, lanolin derivatives quaternary ammonium and the like.

Preferred ambivalent surfactants can comprise carboxy betaine type, amide betaine type, hydroxy sulfo betaine type, phosphpobetaine type, aminocarboxylic acid, imidazoline derivatives type, amide amine type and the like.

Preferred organic and inorganic dyes can comprise silicic acid, anhydrous silicic acid, magnesium silicic acid, talc, ceracyte, mica, kaolin, bengala, clay, bentonite, titan film mica, oxy chlorine bismuth, zirconium oxide, magnesium oxide, zinc oxide, titan oxide, aluminum oxide, calcium sulfate, barium sulfate, magnesium sulfate, calcium carbonate, magnesium carbonate, ferrous oxide, chromium oxide, chromium hydroxide, calamine, carbon black and combinations thereof as an inorganic dyes; polyamide, polyester, polypropylene, polystyrene, polyurethane, vinyl resin, urea resin, phenol resin, fluoride resin, silicone resin, acryl resin, melamine resin, epoxy resin, polycarbonate resin, divinyl benzene-styrene copolymer, silk powder, cellulose, CI pigment yellow, CI pigment orange as an organic dyes; and their complex etc.

Preferred organic powders can comprise metal soaps such as calcium stearate; alkyl phosphonate metal salt such as sodium zinc cetylic acid, zinc laurylic acid, calcium laurylic acid; acylamino acid polyvalent metal salt such as calcium N-lauroyl-β-alanine, zinc N-lauroyl-β-alanine, calcium N-lauroyl-glycine etc.; amide sulfonic acid polyvalent metal salt such as calcium N-lauroyl-taurine, calcium N-palmitoyl-taurine; N-acyl basic amino acid such as Nε-lauroyl-L-lysine, Nε-palmitoyl-lysine, Nα-palmitoyl ornitine, Nα-lauroyl arginine, hardened lanolin fatty acid acyl arginine and the like; N-acylpolypeptide such as N-lauroylglycyl glycine; α-amino fatty acid such as α-amino caprylic acid, α-amino lauric acid and the like; polyethylene, polypropylene, nylon, polymethylmethacrylate, polystyrene, divinylbenzene-styrene copolymer, ethylene tetrafluoride and so on.

Preferred ultraviolet absorbing agents can comprise paraminobenzoic acid, paraamionoethyl benzoate, paramino amyl benzoate, paramino octyl benzoate, ethyleneglycol salicylate, phenyl salicylate, octyl salicylate, benzyl salicylate, butylphenyl salicylate, homomentyl salicylate, benzyl cinnamic acid, paramethoxy 2-ethoxy ethyl cinnamic acid, paramethoxy octyl cinnamic acid, diparamethoxy mono-2-ethylhexane glyceryl cinnamic acid, paramethoxy isopropyl cinnamic acid, diisopropyl-diisopropyl cinnamate ester mixture, urokanic acid, ethyl urokanic acid, hydroxy methoxy benzophenone, hydroxymethoxy benzophenone sulfonic acid and the salt thereof, dihydroxy methoxy benzophenone, dihydroxy methoxy benzophenone disulfonate Na, dihydroxy benzophenone, tetrahydroxybenzophenone, 4-tert-butyl-4′-methoxydibenzoylmethane, 2,4,6-trianilino-p-(carbo-2′-ethylhexyl-1′-oxy)-1,3,5-triazine, 2-(2-hydroxy-5-methylphenyl)benzotriazole and the like.

Preferred preservatives can comprise hinokitiol, trichloric acid, trichlorohydroxydiphenylether, chlorohexidine glucuronate, phenoxyethanol, resorcine, isopropylmethylphenol, azulene, salicylic acid, zinc pilithione, benzalconium HCl, photosensitizer 301, mononitroguaiacol Na, undecylenic acid etc.

Preferred antioxidants can comprise butylhydroxyanisole, propyl gallate, ellisorbate and the like.

Preferred pH controllers can comprise citric acid, sodium citrate, malic acid, sodium malate, fumaric acid, sodium fumaric acid, succinic acid, sodium succinic acid, sodium hydroxide, sodium hydrogen phosphate and the like.

Preferred alcohols can comprise cetyl alcohol etc.

Furthermore, other ingredients may be added to any of the above-described compositions. In one embodiment, the amount of the other ingredients ranges from 0.01 to 5%, more preferably, 0.01 to 3% in that of total composition.

The above-described ingredients such as water-soluble vitamin, lipid soluble vitamin, peptide polymer, polysaccharide polymer, sphingolipid, seaweed extract and other ingredients, can be obtained by conventional methods disclosed in the literature (e.g., see Matsumoto Mithio, Manual for the development of transdermal applied preparations. Seisi Press, 1^{st} Ed., 1985).

The hardy kiwifruit preparations of the present invention can be used safely. They are non-toxic to animals and exhibit no substantial adverse effects.

In accordance with the present invention, any of the above-identified compositions can be formulated with the hardy kiwifruit preparations of the present invention (including any part of the fruit, the whole fruit, the stem, the leaf, the bark, or the root, and including any preparation or extract thereof, including dried preparations, non-extracted but processed preparations, fresh fruit, fruit juice, or any extract or fraction thereof), using any suitable dose or amount of the hardy kiwifruit preparation that is sufficient to achieve the desired biological activity for the hardy kiwifruit preparation as described above, when administered one or more times over a suitable time period. Suitable amounts or doses can vary depending upon the goal of the administration or the condition or the disease being treated, and also on the weight of the subject, severity, drug form, route and period of administration, and may be chosen by those skilled in the art.

A suitable amount or dose of the hardy kiwifruit preparation of the present invention can include, in one embodiment, an amount of from about 0.1 g to about 10 g per kg body weight of the patient, and preferably, from about 1 to 3 g per kg by weight/day of the inventive hardy kiwifruit or extract of the present invention. The dose may be administered once per day, several times per day, or in longer increments (e.g., every few days, weekly, monthly, etc.), as desired. In terms of the compositions described herein, the amount of hardy kiwifruit preparation of the present invention should be present between about 0.01% to 100% by weight, and preferably between about 0.01% and about 95% by weight, and more preferably 0.5 to 80% by weight based on the total weight of the composition, including any amount in between 0.01% and 100%, in 0.01% increments. In one embodiment, a pharmaceutical composition of the present invention can contain about 0.01-50% by weight of the hardy kiwifruit preparation of the present invention based on the total weight of the composition.

In one embodiment, an extract or other preparation of the hardy kiwifruit preparation of the present invention may be provided in any composition at from about 20% to 90% highly concentrated liquid, powder, or granule, including any increment between 20% and 90%, in 1% increments.

The ratio of additional components in the composition may generally range from about 0 to 20 w/w % per 100 w/w % of the composition, including any increment between 0 and 100 w/w %, in 1% increments.

In one embodiment, a cosmetic composition comprises the hardy kiwifruit preparation of the present invention in an amount of from about with 0.01 to 30%, and more preferably, 0.01 to 5% by the weight based on the total weight of the composition, including any increment between 0.01% and 30%, in 0.01% increments.

In another embodiment, when a composition comprising the hardy kiwifruit preparation of the present invention that is added to food, a food additive or a beverage, can be provided in an amount ranging from about 0.1 to 95 w/w %, preferably 1 to 80 w/w % of total weight of food, additive or beverage, including any increment between 0.1 and 95 w/w %, in 0.1 w/w % increments, or about 1 to 30 g per 100 ml and preferably 3 to 10 g per 100 ml, including any increment between 1 g per 100 ml and 30 g per 100 ml, in 1 g increments, of a health beverage composition.

In one embodiment, a health food of the present invention comprises the hardy kiwifruit preparation of the present invention as 0.01 to 80%, preferably 1 to 50% by weight based on the total weight of the composition, including any increment between 0.01% and 80%, in 0.1% increments.

In one embodiment, a health food beverage comprises the hardy kiwifruit preparation of the present invention in an amount of from about 0.01 to about 20% by weight of the total weight of the composition, including any increment between 0.01% and 20%, in 0.01% increments. Additional components may include: amino acids 0.001 to 5% by weight, vitamins 0.001 to 2% by weight, sugars 0.001 to 20% by weight, organic acids 0.001 to 10% by weight, sweetener and flavors of a suitable amount. Providing that the health beverage composition of present invention contains the above-described hardy kiwifruit preparation of the present invention as an essential component, there is no particular limitation on the other liquid components, wherein the other component can be various sweeteners and/or flavor enhancers, such as may be added to a conventional beverage. Examples of such sweeteners or flavor enhancers include, but are not limited to, conventional or reduced calorie sweeteners, including monosaccharides such as glucose, fructose etc; disaccharides such as maltose, sucrose etc; conventional sugars such as dextrin, cyclodextrin; and sugar alcohols such as xylitol, and erythritol etc. Additional sweeteners include natural sweeteners such as taumatin, stevia extract, levaudioside A, glycyrrhizin and derivatives thereof, and reduced calorie sweeteners such as saccharin, sucralose, aspartame and derivatives thereof. The amount of the above-described sweeteners or flavor enhancers generally ranges from about 1 to 20 g, and preferably 5 to 12 g in the ratio of 100 ml of the beverage composition, including any increment between 1 g and 20 g per 100 ml, in 1 g increments.

A food additive can be added to a food by deposition, spray, or mixing. The amount of the additive with respect to the total composition may generally range from about 0.01 to 20 w/w % per 100 w/w % of the present composition, including any increment between 20 w/w % and 100 w/w %, in 1 w/w % increments. Food additives can also be mixed with a feed, such as an animal feed, in an amount of from about 5 to 100 g per 1 kg by weight based on the total dried weight of the feed, including any increment between 5 g and 100 g per 1 kg by weight, in 1 g increments.

Accordingly, it is an object of the present invention to provide a method to selectively regulate Th1 and Th2 immune responses in a patient by administering or providing a composition, including a pharmaceutical composition, a nutraceutical composition, a food additive, a health food (including a beverage or a food material), or a cosmetic composition, comprising, consisting essentially of, or consisting of, any of the hardy kiwifruit preparations described herein (including any part of the fruit, the whole fruit, the stem, the leaf, the bark, or the root, and including any preparation or extract or concentrate thereof, including dried preparations, non-extracted but processed preparations, fresh fruit, fruit juice, or any extract or concentrate or fraction thereof), and in one embodiment, a crude extract, a total water-soluble extract, or an ethyl acetate extract of the hardy kiwifruit, as an active ingredient. The compositions of the invention are described in detail above. In particular, the administration or provision of the composition results in at least one of the following biological activities: (a) reduces the number of IgE-producing B cells in a patient; (b) reduces the amount of IgE produced in a patient (e.g., in the serum or plasma); (c) decreases production and/or levels of at least one Th2 cytokine (e.g., IL-4, IL-5, IL-10); (d) increases the level of at least one Th1 cytokine (e.g., IL-12, IFN-γ); (e) decreases the level of expression of the transcription factor, GATA-3; (f) increases the level of expression of the transcription factor T-bet; (g) increases the level of expression of the transcription factor NFATc2; (h) increases the number of IgG2a-producing B cells in a patient; (i) increases the amount of IgG2a produced in a patient; (j) enhanced production or activity of Th1 T lymphocytes (e.g., CD4+, IFN-γ+), particularly at a site of inflammation; (k) decreased production or activity of Th2 T lymphocytes (e.g., CD4+, IL-4+), particularly at a site of inflammation; (1) reduces the number of IgG1-producing B cells in a patient; (m) reduces the amount of IgG1 produced in a patient; and/or (n) reduces the level of or production of at least one leukotriene in the patient.

A preferred method of the present invention includes a method to reduce leukotriene production in a patient, thereby treating or ameliorating at least one symptom of a condition or disease associated with leukotrienes in a patient. The method comprises administering to said mammal an effective amount of any of the hardy kiwifruit preparations described herein (including any part of the fruit, the whole fruit, the stem, the leaf, the bark, or the root, and including any preparation or extract or concentrate thereof, including dried preparations, non-extracted but processed preparations, fresh fruit, fruit juice, or any extract or concentrate or fraction thereof), and in one embodiment, a crude extract, a total water-soluble extract, or an ethyl acetate extract of the hardy kiwifruit, together with a pharmaceutically acceptable carrier thereof. Preferably, the administration of the composition of the invention results in the reduction of leukotriene production or levels in the patient. Diseases and conditions associated with leukotrienes include, but are not limited to, asthma, food allergy, allergic rhinitis, chronic urticaria, and allergic dermatitis. In this embodiment, preferred routes of administration include oral, inhaled and topical administration, in addition to systemic routes of administration.

The method described above can be used for the prevention and/or treatment of any disease or condition in which regulation of the immune response in the manner described herein would be, or could be predicted to be, beneficial to a patient.

It is therefore an object of the present invention to provide a method of treating and/or preventing allergic disease and non-allergic inflammatory disease in a mammal, comprising administering to said mammal an effective amount of any of the hardy kiwifruit preparations described herein (including any part of the fruit, the whole fruit, the stem, the leaf, the bark, or the root, and including any preparation or extract or concentrate thereof, including dried preparations, non-extracted but processed preparations, fresh fruit, fruit juice, or any extract or concentrate or fraction thereof), and in one embodiment, a crude extract, a total water-soluble extract, or an ethyl acetate extract of the hardy kiwifruit, together with a pharmaceutically acceptable carrier thereof.

According to the present invention, allergic diseases can include, but are not limited to, asthma, allergic bronchopulmonary aspergillosis, allergic bronchitis bronchiectasis, hypersensitivity pneumonitis, allergic sinusitis, anaphylaxis, allergic rhinitis, allergic conjunctivitis, allergic dermatitis, atopic dermatitis, contagious dermatitis, chronic urticaria, insect allergies, food allergies and drug allergies.

In one embodiment, the allergic disease is atopic dermatitis. In one aspect of this embodiment, in addition to administration of the composition comprising, consisting essentially of or consisting of a hardy kiwifruit preparation of the invention, the patient is concomitantly treated with a conventional therapy for atopic dermatitis. In this embodiment of the invention, the hardy kiwifruit preparation is administered most preferably by oral or topical administration, although the invention is not limited to such routes of administration.

In another embodiment, the allergic disease is asthma. In one aspect of this embodiment, in addition to administration of the composition comprising, consisting essentially of or consisting of a hardy kiwifruit preparation of the invention, the patient is concomitantly treated with a conventional therapy for asthma. In this embodiment of the invention, the hardy kiwifruit preparation is administered most preferably by oral or inhaled administration, although the invention is not limited to such routes of administration.

According to the present invention, non-allergic skin inflammation diseases can include, but are not limited to, various skin troubles caused by inflammation such as pimples, acne and the like. The above-described cosmetic compositions comprising any of the hardy kiwifruit preparations described herein (including any part of the fruit, the whole fruit, the stem, the leaf, the bark, or the root, and including any preparation or extract or concentrate thereof, including dried preparations, non-extracted but processed preparations, fresh fruit, fruit juice, or any extract or concentrate or fraction thereof), and in one embodiment, a crude extract, a total water-soluble extract, or an ethyl acetate extract of the hardy kiwifruit, are useful for preventing, treating, and/or improving skin inflammation in a patient.

Other non-allergic inflammatory diseases that can be prevented or treated using the compositions and methods described herein include, but are not limited to, various dermatitis conditions, systemic lupus erythematosus (SLE), retinal inflammation, gastritis, retinopathy, hepatitis, enteritis, pancreatitis, nephritis and similar conditions where reduction of a Th2 type immune response and/or enhancement of a Th1 type immune response would be beneficial.

It is another object of the present invention to provide a method of treating and/or preventing a viral infection in a mammal, comprising administering to said mammal an effective amount of any of the hardy kiwifruit preparations described herein (including any part of the fruit, the whole fruit, the stem, the leaf, the bark, or the root, and including any preparation or extract or concentrate thereof, including dried preparations, non-extracted but processed preparations, fresh fruit, fruit juice, or any extract or concentrate or fraction thereof), and in one embodiment, a crude extract, a total water-soluble extract, or an ethyl acetate extract of the hardy kiwifruit, together with a pharmaceutically acceptable carrier thereof.

Preferred viruses from which to protect a mammal by preventing or treating a viral infection include, but are not limited to, Coxsackie viruses, cytomegaloviruses, Epstein-Barr viruses, flaviviruses, hepatitis viruses, herpes viruses, influenza viruses, measles viruses, mumps viruses, papilloma viruses, parainfluenza viruses, parvoviruses, rabies viruses, respiratory syncytial viruses, retroviruses and varicella viruses.

Among these viruses, retroviruses, herpes viruses, and hepatitis viruses are more preferred, with leukemia, lymphotrophic, sarcoma and lentiviruses being even more preferred, as are other immunodeficiency or tumor viruses. Particularly preferred lymphotrophic viruses from which to protect a mammal by preventing or treating a viral infection include T-lymphotrophic viruses, such as human T-cell lymphotrophic viruses (HTLVs, such as HTLV-I and HTLV-II), bovine leukemia viruses (BLVs) and feline leukemia viruses (FLVs). Particularly preferred lentiviruses include human (HIV), simian (SIV), feline (FIV) and canine (CIV) immunodeficiency viruses, with HIV-1 and HIV-2 being even more preferred.

It is another object of the present invention to provide a method of treating and/or preventing a cancer in a mammal, comprising administering to said mammal an effective amount of any of the hardy kiwifruit preparations described herein (including any part of the fruit, the whole fruit, the stem, the leaf, the bark, or the root, and including any preparation or extract thereof, including dried preparations, non-extracted but processed preparations, fresh fruit, fruit juice, or any extract or fraction thereof), and in one embodiment, a crude extract, a total water-soluble extract, or an ethyl acetate extract of the hardy kiwifruit, together with a pharmaceutically acceptable carrier thereof.

Cancers to be treated or prevented using the methods and compositions of the present invention include, but are not limited to, melanomas, squamous cell carcinoma, breast cancers, head and neck carcinomas, thyroid carcinomas, soft tissue sarcomas, bone sarcomas, testicular cancers, prostatic cancers, ovarian cancers, bladder cancers, skin cancers, brain cancers, angiosarcomas, hemangiosarcomas, mast cell tumors, primary hepatic cancers, lung cancers, pancreatic cancers, gastrointestinal cancers, renal cell carcinomas, hematopoietic neoplasias, and metastatic cancers thereof.

In any of the above methods to treat or prevent a disease or condition, the hardy kiwifruit preparation can be administered in conjunction with another therapy or composition that is useful for treating the particular condition. In these embodiments, the hardy kiwifruit can be considered to be an adjunct to a conventional therapy, to enhance the improvement, recovery, or amelioration of symptoms in the patient. Particularly preferred types of conventional agents or therapies that can be used together with a hardy kiwifruit preparation of the invention include, but are not limited to, antihistamines (any type, including systemic, topical, inhaled), antibodies (e.g., anti-IgE, anti-IL-10), antibiotics, cyclosporins, antimycotics, respiratory function controllers, analgesics, β-agonists (long or short acting), leukotriene modifiers (inhibitors or receptor antagonists), cytokine or cytokine receptor antagonists, phosphodiesterase inhibitors, sodium cromoglycate, nedocrimil, caffeine, theophylline, carbobenzoxy beta-alanyl taurine, inhibitors of T cell function and other anti-inflammatory agents.

In the method of the present invention, compositions can be administered or provided to any member of the Vertebrate class, Mammalia, including, without limitation, primates, rodents, livestock, horses and domestic pets. Preferred patients to protect are domestic pets (e.g., dogs, cats) and humans, with humans being particularly preferred. All modes of administration are contemplated. According to the present invention, the terms “patient”, “subject” and “individual” can be used interchangeably.

Administration routes include in vivo, in vitro and ex vivo routes. Ex vivo refers to performing part of the regulatory step outside of the patient. In vivo routes include, but are not limited to, intravenous administration, intraperitoneal administration, intramuscular administration, intranodal administration, intracoronary administration, intraarterial administration (e.g., into a carotid artery), subcutaneous administration, transdermal delivery, intratracheal administration, intraarticular administration, intraventricular administration, inhalation (e.g., aerosol), intracranial, intraspinal, intraocular, aural, intranasal, oral, pulmonary administration, impregnation of a catheter, intracutaneous, intrathecal, epidural, intracerebroventricular injection, and direct injection into a tissue. In one embodiment of the present invention, a composition is administered by a parenteral route (e.g., subcutaneous, intradermal, intravenous, intramuscular and intraperitoneal routes). Intravenous, intraperitoneal, intradermal, subcutaneous and intramuscular administrations can be performed using methods standard in the art. Aural delivery can include ear drops, intranasal delivery can include nose drops or intranasal injection, and intraocular delivery can include eye drops. Aerosol (inhalation) delivery can also be performed using methods standard in the art (see, for example, Stribling et al., Proc. Natl. Acad. Sci. USA 189:11277-11281, 1992, which is incorporated herein by reference in its entirety). For example, in one embodiment, a composition or vaccine of the invention can be formulated into a composition suitable for nebulized delivery using a suitable inhalation device or nebulizer. Oral delivery can be performed by complexing a composition of the present invention to a carrier capable of withstanding degradation by digestive enzymes in the gut of an animal, for example, as tablets or capsules, as well as being formulated into food and beverage products. Examples of such carriers, include plastic capsules or tablets, such as those known in the art. Direct injection techniques are particularly useful for site-specific administration of a compound. Oral delivery or topical delivery are particularly preferred routes of delivery or administration according to the present invention. Routes of administration that modulate mucosal immunity are useful in the treatment of viral infections and some allergic conditions. Such routes include bronchial, intradermal, intramuscular, intranasal, other inhalatory, rectal, subcutaneous, topical, transdermal, vaginal and urethral routes.

The present invention is more specifically explained by the following examples. However, it should be understood that the present invention is not limited to these examples in any manner.

### EXAMPLES

### Example 1

The following example demonstrates that at least two specific extracts prepared from A. arguta, denoted PG102T and PG102E, contain inhibitory activity on the production of IgE as well as the ability to regulate selective Th1 and Th2 cytokines.

### Materials and Methods

Mice. BALB/c female mice (6 wks old) were obtained from Daehan Biolink, Co. Ltd. (Korea), kept in an air-conditioned and pathogen-free room and acclimated for at least 1 wk. All experimental procedures mentioned below were performed in accordance with the institutional animal care and use guidelines of the Animal Experimental Center at Seoul National University.

Preparation of various extracts from A. Arguta. The hardy kiwifruits used in this study were purchased from a farm specializing in the cultivation of this fruit (Hurstberry Co. Ltd., Oregon, USA) and their identity was kindly confirmed by Dr. Ella I. Kolbasina (The Moscow Branch of Vavilov Plant Cultivation Research Institute, Russia). The dried fruit (10 g) was extracted three times by heating in distilled water (DW). It was then concentrated, freeze-dried and dissolved in DW to produce PG102T at 100 mg/ml. PG102T dissolved in DW was extracted successively with chloroform, ethyl acetate and n-butanol resulting in PG102C, PG102E, and PG102B, respectively. The remaining aqueous layer was called PG102W. Each solvent-soluble fraction and the final aqueous residue were filtered, concentrated, freeze-dried, and dissolved at concentrations of 100 mg/ml. All preparations were stored at −80° C. until such time as needed.

Bioassay in U266B1 cells. The IgE inhibitory effects in LPS-stimulated human B lymphoblastoma cells, U266B1 (ATCC, Manassas, Va.), were measured as described by Kim et al. with a slight alteration (Kim et al., Phytother Res 2001; 15:572-6). The level of human IgE in culture supernatants was detected by human IgE ELISA (total human IgE; AlerChek, Portland, Me.) and cell viability was assessed by a LDH detection kit (Takara Bio, Japan).

In vitro effect of PG102 on cytokine production through the recall response in OVA-stimulated splenocytes. Mice (7 wks old) were individually immunized and later boosted by intraperitoneal (i.p.) injections of 20 μg of ovalbumin (OVA; grade V; Sigma, St. Louis, Mo.) emulsified in 2.25 mg of aluminum hydroxide (ImjectAlum; Pierce, Rockford, Ill.) on day 0 and day 14, respectively. Non-sensitized (naïve) mice did not receive any reagent. On day 24, both OVA-sensitized and naïve mice were sacrificed (n=5/group), and each spleen was then isolated to study the production of cytokines in splenocytes using the recall response as described by Shibata et al. (Yoshimi et al., J Immunol 2000; 164; 1314-21). Briefly, isolated splenocytes were seeded to a 24 well culture plate, and the final concentration was adjusted to 5×10⁶ cells/ml/well. Splenocytes were incubated with OVA at 100 μg/ml in the presence of PG102T (1 mg/ml), PG102C, PG102E, PG102B, PG102W (all at 0.1 mg/ml) or media as a control for 3 days. Following incubation, the culture supernatants were collected to detect the level of cytokines (IL-4, IL-5, IL-12 and IFN-γ) using ELISA kits (Endogen, Cambridge, Mass.). The virtually identical procedure was used to determine the specific activity of PG102T and PG102E.

Measurement of cytokines and immunoglobulins in OVA-sensitized mice. Mice were immunized and boosted as described above. To assess the in vivo effects of PG102 preparations on OVA-induced allergic responses, OVA-sensitized mice (n=10/group) were orally treated with PG102T (15 mg/kg/day) or PG102E (1.5 mg/kg/day) with dexamethasone (DEX, 0.5 mg/kg/day) or DW (100 μL/mouse/day) as a control, once a day from day 14 to day 24. Naïve mice were orally treated with DW. On day 21, blood was obtained from individual mice by eye-bleeding and isolated plasma samples were kept at −80° C. until time of use. The level of total IgE was measured by way of a mouse IgE detection kit (Shibayagi, Gunma, Japan). The levels of total IgG subtypes and OVA-specific Ig isotypes were determined by the sandwich ELISA method (Hirano et al., J Immunol Methods 1989; 119:145-50). For the measurement of cytokine production, splenocytes were prepared from animals on day 24, resuspended in culture medium (RPMI-1640 containing 10% FBS), seeded onto a 24 well plate (5×10⁶ cells/ml/well) and incubated with OVA only at 100 μg/ml for 3 days. Splenocytes isolated from naïve mice were cultured in the absence of OVA. The levels of IL-4, IL-5, IL-10, IL-12, IL-13 and IFN-γ in supernatants were detected by ELISA (Endogen and R&D Systems, Minneapolis, Minn.).

Immunostaining analysis. Splenocytes were exposed to GolgiStop (PharMingen, San Diego, Calif.) as an intracellular protein transport inhibitor for 4 h and prepared for the detection of IL-4 or IFN-γ-producing cells. Cells were fixed, permeabilized, and incubated with PE or FITC-conjugated antibodies specific to mice CD4, IL-4 or IFN-γ as described by Kyoko et al. (Kyoko et al., J Derm Science 2002; 29:19-25). For the analysis of IgE products, cells were incubated with PE-conjugated anti-mouse CD19, followed by FITC-conjugated anti-mouse IgE (an from PharMingen). Cells were then examined by analyzing the gated lymphocytes in splenocytes using a FACSort analyzer and Cell Quest software (Becton Dickinson, San Jose, Calif.). For confocal microscopic analysis, splenocytes were cultured with OVA on cover slips for 2 days, fixed, permeabilized, and stained using FITC-conjugated antimouse IgE and PE-conjugated anti-mouse CD19, and finally observed by the MRC-1024 Laser Scanning Confocal Image System (Bio-Rad Laboratories Inc., Hercules, Calif.) as described by Semper et al. (Semper et al., J Allergy Clin Immunol 2003; 112:141-9).

Western blotting. Splenocytes from the individual group of mice were cultured with OVA for 2 days, collected (10⁷ cells/group), and then lysed for the preparation of protein samples. Immunoblotting was performed using antibodies specific to mouse GATA-3, T-bet, NFATc2 (Santa Cruz Biotechnology, Santa Cruz, Calif.) or β-actin (Sigma) as a loading control.

RNA preparation and quantitative real-time PCR. Total RNA was isolated from splenocytes cultured with OVA for 2 days using TRIzol Reagent (GIBCOBRL, Carlsbad, Calif.). Isolated total RNA was used in reverse transcription (RT)-PCR by the AMV RT System (Roche, Mannheim, Germany), followed by quantitative real-time PCR using the ABI PRISM 7700 Sequence Detection System (Applied Biosystems, Foster City, Calif.). Forward and reverse primer sets for mouse genes were designed using Primer Express Software (Applied Biosystems) and the nucleotide sequences are as follows:

| | | |
|---|---|---|
| GATA3 | | |
| forward | 5′-CCTCGGCCATTCGTACATG-3′ | (SEQ ID NO: 1) |
| reverse | 5′-CGTAGTAGGACGGGACGTGG-3′ | (SEQ ID NO: 2) |
| T-bet | | |
| forward | 5′-TGTGGATGTGGTCTTGGTGG-3′ | (SEQ ID NO: 3) |
| reverse | 5′-ATAAGCGGTTCCCTGGCAT-3′ | (SEQ ID NO: 4) |
| NFATc2 | | |
| forward | 5′-GCACATAAGGCCATCAGCTCA-3′ | (SEQ ID NO: 5) |
| reverse | 5′-TCGCCAGAGAGACTGGCAA-3′ | (SEQ ID NO: 6) |
| GAPDH | | |
| forward | 5′-TGCAGTGGCAAAGTGGAGATT-3′ | (SEQ ID NO: 7) |
| reverse | 5′-TTGAATTTGCCGTGAGTGGA-3′ | (SEQ ID NO: 8) |

The differences in mRNA levels of cellular genes between each group of mice were calculated from conversion of the ΔCt value.

Statistics. Data were expressed as mean±SEM, and differences between mean values were analyzed by unpaired Student's t test. P values less than 0.05 or 0.01, which were calculated as one-tailed P values, were considered to be statistically significant.

### Results

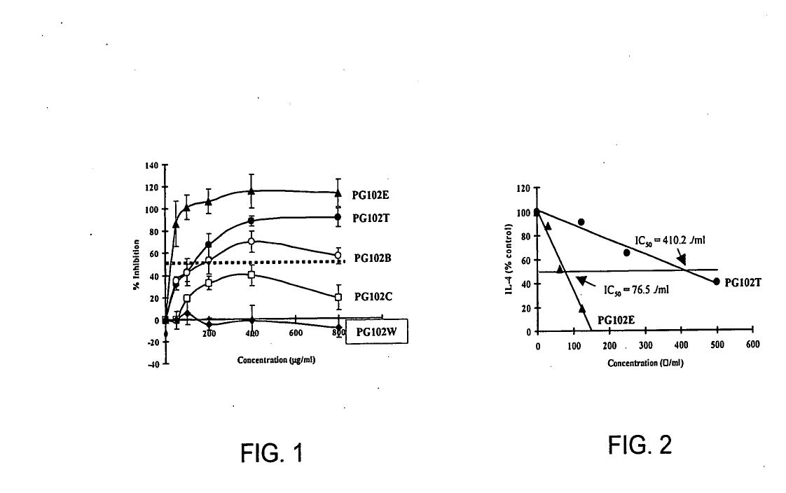

Effects of various preparations from A. arguta in LPS-stimulated human U266B1 B cell line and OVA-stimulated mouse splenocytes. The inventors initially prepared a total water-soluble extract (PG102T) from A. arguta, and extracted it successively with chloroform (PG102C), ethyl acetate (PG102E), n-butanol (PG102B) and water (PG102W), to obtain four fractions of different polarity. The effects of PG102T and these four fractions on IgE production were tested using human B lymphoblastoma U266B1 cells (FIG. 1). Four preparations, except for PG102W, were found to inhibit the production of IgE from LPS-stimulated U266B1 cells. PG102E exhibited the highest inhibitory activity, displaying a 50% inhibitory effect at 25 μg/ml (IC₅₀). PG102T was also active, its IC₅₀ value being 126 μg/ml. In all concentrations tested in this assay, neither PG102T nor PG102E showed any cytotoxic effects on U266B1 cells.

In addition, the inventors studied the effects of various preparations from A. arguta on the production of cytokines involved in Th1 and Th2 pathways using the recall response model. Mice were immunized with OVA on day 0 and boosted on day 14. Ten days later, mice were sacrificed and spleens were taken to isolate splenocytes. Splenocytes derived from OVA-sensitized mice were stimulated with OVA and cultured for 3 days in the presence of five preparations of PG102. As shown in Table I, when cells were grown in the presence of OVA, the levels of IFN-γ, IL-4 and IL-5 were increased to a hundred picograms or nanograms. In the case of IL-12, the background level was high, but OVA stimulation decreased the level by approximately 3-fold to 415±48 pg/ml. Treatment with PG102T resulted in an almost 150% increase in the level of IL-12. IFN-γ behaved differently. Splenocytes from naïve animals produced undetectable levels of IFN-γ, but OVA-stimulated splenocytes secreted almost 2.5 ng/ml. Treatment with PG102T decreased the level of IFN-γ by 40%. When OVA-stimulated splenocytes were grown in the presence of PG102T, the levels of IL-4 and IL-5 were decreased by 62% and 39%, respectively.

TABLE I
In vitro effects of PG102 on cytokine production by splenocytes

| Treatment | IL-12 | | IFN-γ | | IL-4 | | IL-5 | |
|---|---|---|---|---|---|---|---|---|
| Naïve | 1277 ± 275 | (308) | ND | | 8 ± 1 | (2) | 15 ± 3 | (2) |
| Ova-sensitized | | | | | | | | |
| Media | 415 ± 48 | (100) | 2525 ± 1474 | (100) | 324 ± 15 | (100) | 847 ± 137 | (100) |
| PG102T | 622 ± 56 | (150)* | 1478 ± 1214 | (59) | 124 ± 34 | (38)** | 523 ± 30 | (61) |
| PG102C | 342 ± 12 | (82) | ND | | 90 ± 57 | (28)* | 84 ± 26 | (10)* |
| PG102E | 927 ± 218 | (223)* | 703 ± 406 | (28) | 70 ± 5 | (22)** | 345 ± 166 | (41)* |
| PG102B | 575 ± 136 | (139) | 666 ± 366 | (26) | 290 ± 44 | (90) | 623 ± 20 | (74) |
| PG102W | 501 ± 128 | (121) | 897 ± 592 | (36) | 280 ± 73 | (87) | 676 ± 61 | (80) |

All splenocytes from OVA-sensitized mice were stimulated again with OVA during the culture. Values are expressed as means ± SEM for five animals.
*P < 0.05 and
**P < 0.01, vs. OVA-stimulated splenocytes treated with media alone (Student's T-test).
ND = not detectable.

PG102E, which showed the highest inhibitory effect on IgE production in the above experiment, also reduced the levels of IL-4 and IL-5 by 78% and 59%, while inducing the level of IL-12 by 220%. In contrast, PG102C reduced the level of all cytokines measured in this study due to its cytotoxic effect on splenocytes. Both PG102B and PG102W increased the level of IL-12 and decreased that of IFN-γ, IL-4 and IL-5, but not in a statistically significant manner. Taken together, these results indicated that PG102T and PG102E might contain the compound(s) that inhibits the production of IgE and controls the expression of selective Th1 and Th2 cytokines. Based on the above data, the inventors chose to use two preparations, PG102T and PG102E, for further in vivo studies.

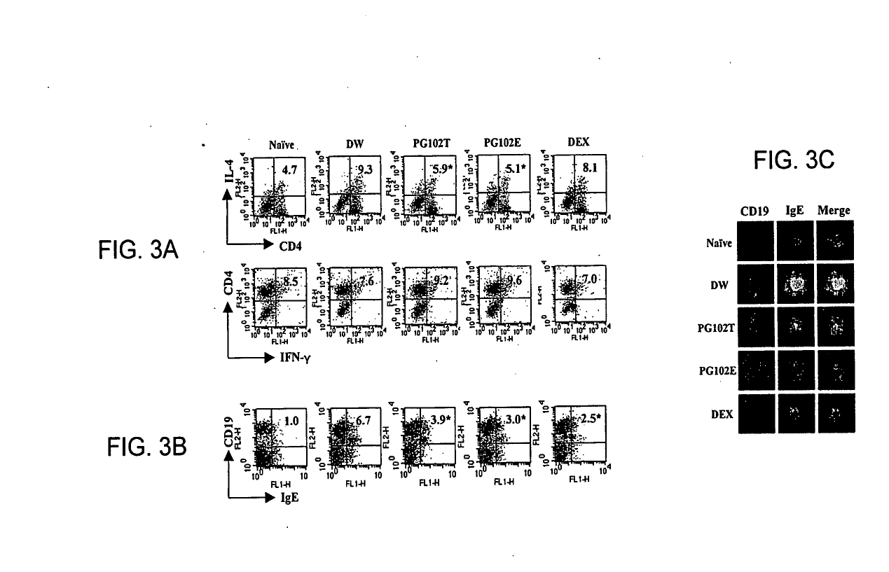

Determination of specific activity of PG102T and PG102E. Based on their pleiotropic activities on the immune system, PG102T and PG102E as derived from a plant source were thought to contain more than one active compound. Therefore, to quantitatively perform further experimentation, the inventors developed a reliable bioassay based on the inhibitory activity of PG102 on the production of IL-4 in OVA-stimulated splenocytes as described above. When cells were stimulated with OVA, the level of IL-4 was induced from an undetectable level to a few hundred picograms. With the presence of PG102T and PG102E, the production of IL-4 was inhibited in a dose-dependent manner (FIG. 2). In all concentrations tested in this assay, neither PG102T nor PG102E demonstrated any cytotoxic effects. The concentration of PG102T with 50% inhibitory activity (IC₅₀) was 806.9 μg/ml and that of PG102E was 91.8 μg/ml. The IC₅₀ value of each preparation was defined as one activity unit, and total units were obtained. When the specific activity of PG102T and PG102E was calculated using total units and yield, PG102T and PG102E contained specific activity of 1.2 units/mg and 10.9 units/mg, respectively. This method was used for the quality control of experimental samples from A. arguta.

Effects of PG102T and PG102E on production of Th1 and Th2 cytokines in the OVA-sensitized murine model. To confirm the above in vitro data in the animal model, effects of PG102T and PG102E were tested on the production of various cytokines involved in the modulation of Th1 and Th2 pathways using the OVA-sensitized murine model. Mice were immunized with OVA on day 0 and boosted on day 14. After boosting, animals were orally dosed with PG102T (15 mg/kg/day=18 units/kg/day) or PG102E (1.5 mg/kg/day=16.4 units/kg/day) and as a control, DEX (0.5 mg/kg/day) or DW on a daily basis from day 14 to day 24. Naïve mice untreated with OVA were orally fed with DW. The concentrations of PG102T and PG102E used in these experiments are the minimal dosage having maximal activity as per the preliminary dose-response experiment. On day 24, mice were sacrificed, and spleens were isolated to prepare splenocytes. Splenocytes from each group of mice were incubated in the presence of OVA for 3 days, and cultured supernatants were collected to measure the level of cytokines (Table II). Compared with naïve splenocytes, the level of IL-12 was decreased in DW-treated mice. However, oral administrations of PG102T and PG102E increased its level by 1.7- and 2.6-fold, respectively. Unlike IL-12, the level of IFN-γ was enhanced by OVA-stimulation, and PG102T or PG102E further increased the level of IFN-γ. Contrary to PG102T and PG102E, DEX suppressed the level of both IL-12 and IFN-γ.

TABLE II
In vivo effects of PG102 on Th1 and Th2 cytokine production by splenocytes

| Sources | IL-12 | | IFNγ | | IL-4 | | IL-5 | | IL-10 | | IL-13 | |
|---|---|---|---|---|---|---|---|---|---|---|---|---|
| Naïve | 1353 ± 201 | (394) | ND | | ND | | ND | | 77 ± 54 | (2) | 23 ± 17 | (1.5) |
| OVA- | | | | | | | | | | | | |
| sensitized | | | | | | | | | | | | |
| DW | 343 ± 33 | (100) | 1023 ± 19 | (100) | 460 ± 42 | (100) | 1345 ± 68 | (100) | 3254 ± 298 | (100) | 1564 ± 132 | (100) |
| PG102T | 584 ± 136 | (170)* | 1970 ± 142 | (193)** | 258 ± 31 | (56)* | 913 ± 79 | (68)* | 1812 ± 296 | (56)** | 1220 ± 96 | (78) |
| PG102E | 889 ± 68 | (259)** | 1778 ± 237 | (174)* | 164 ± 4 | (36)* | 412 ± 45 | (31)** | 1591 ± 114 | (49)** | 976 ± 304 | (62) |
| DEX | 227 ± 44 | (66) | 224 ± 118 | (22) | 28 ± 21 | (6)* | 160 ± 159 | (12)* | 392 ± 157 | (12)** | 912 ± 371 | (58) |

All splenocytes from OVA-sensitized mice were stimulated again with OVA. Values are expressed as mean ± SEM for ten animals.
*P < 0.05 and
**P < 0.01 vs. DW-treated mice (Student's T-test).
ND = not detectable.

The level of all Th2 cytokines tested in this study was highly increased in OVA-stimulated splenocytes. However, treatment with PG102T suppressed OVA-mediated overproduction of IL-4, IL-5, and IL-10 by 44%, 32% and 44%, respectively. PG102E also inhibited the level of these three cytokines by 64%, 69% and 51%, respectively. DEX also lowered the concentration of all three cytokines. IL-13 was decreased by either PG102 or DEX, but not in a statistically significant manner Taken together, these results indicated that both PG102T and PG102E can control the production of selective Th1 and Th2 cytokines. Unlike DEX suppressing virtually all cytokines in a non-discriminating manner, PG102T and PG102E appear to have distinctive biological activities that can differentially modulate the production of Th1 and Th2 cytokines.

Effects of PG102T and PG102E on plasma levels of immunoglobulin isotypes. The above results indicated that PG102T and PG102E might down-regulate Th2-mediated overproduction of IgE in vivo. Therefore, it was tested whether oral treatment with PG102T and PG102E in OVA-sensitized mice could control the plasma levels of IgE and other immunoglobulins. On day 21 during the same type of experiment as described above, naïve mice produced about 140 ng/ml of total IgE, but sensitization with OVA increased its level by approximately 20-fold. When animals were treated with PG102T and PG102E, the plasma level of total IgE was decreased by about 2-fold. The capability of PG102T or PG102E to down-regulate the level of total IgE in plasma was comparable to that of DEX. When the level of various IgG subtypes was measured, administration of both PG102T and PG102E decreased the level of Th2-mediated IgG1, whereas the level of Th1-mediated IgG2a was highly elevated in a statistically significant manner. The level of IgG2b was not significantly affected in all situations (Table IIIA).

A virtually identical result was obtained when the levels of OVA-specific IgE and IgG subtypes were determined (Table IIIB) Oral treatment with PG102T or PG102E decreased the level of OVA-specific IgE and IgG1, while increasing that of OVA-specific IgG2a by more than 2-fold. These results, together with data on various Th1 and Th2 cytokines, indicated that PG102T and PG102E contain compounds that may regulate the Th1 and Th2 balance, eventually resulting in an increase in the level of IgG2a and a decrease in the levels of IgE and IgG1.

TABLE III
Effects of PG102 on plasma levels of total and OVA-specific Immunoglobulin isotypes
A.

| | Total Ig | | | | | | | |
|---|---|---|---|---|---|---|---|---|
| Sources | IgE (ng/ml) | | IgG1 (μg/ml) | | IgG2a (μg/ml) | | IgG2b (μg/ml) | |
| Naïve | 139 ± 23 | (5) | 450 ± 81 | (26) | 46 ± 1 | (159) | 229 ± 52 | (85) |
| OVA-sensitized | | | | | | | | |
| DW | 2783 ± 485 | (100) | 1700 ± 21 | (100) | 29 ± 3 | (100) | 270 ± 36 | (100) |
| PG102T | 1669 ± 319 | (60)* | 1009 ± 443 | (59)* | 67 ± 4 | (231)* | 325 ± 17 | (120) |
| PG102E | 1271 ± 247 | (46)* | 1446 ± 288 | (85) | 44 ± 4 | (152)* | 220 ± 65 | (81) |
| DEX | 1190 ± 317 | (43)* | 1614 ± 36 | (95) | 41 ± 7 | (141) | 313 ± 82 | (116) |

B.

| | OVA-specific Ig (% control of OD) | | | |
|---|---|---|---|---|
| Sources | IgE | IgG1 | IgG2a | IgG2b |
| Naïve | 13 ± 1 | 5 ± 1 | 17 ± 1 | 51 ± 13 |
| OVA-sensitized | | | | |
| DW | 100 | 100 | 100 | 100 |
| PG102T | 29 ± 7** | 76 ± 7* | 217 ± 54* | 104 ± 12 |
| PG102E | 27 ± 8** | 69 ± 9* | 304 ± 53** | 84 ± 9 |
| DEX | 27 ± 8** | 79 ± 11* | 89 ± 25 | 84 ± 9 |

Values show mean ± SEM for ten animals.
*P < 0.05 and
**P < 0.01 vs. DW-treated mice (Student's T-test).
Since OVA-specific antibodies were not available, the relative level of antibodies was calculated as the percentage of control OD.
OD = Optical density.

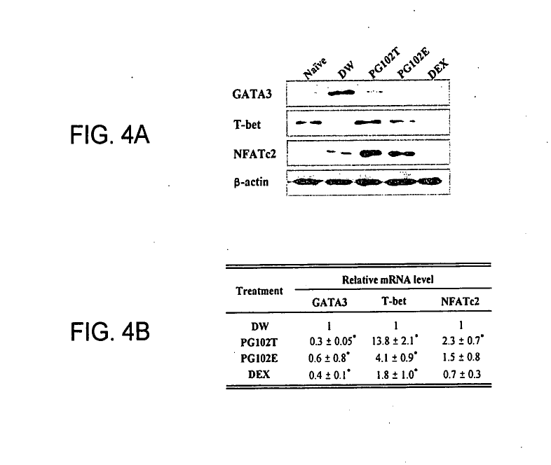

Effects of PG102T and PG102E on T and B cell population in splenocytes. To understand the cellular basis of PG102T and PG102E activities described above, the inventors tested how PG102 administration affected T cell and B cell populations present in splenocytes. Splenocytes were isolated from OVA-sensitized and naïve mice and grown in the presence of OVA for 2 days, followed by FACS and confocal microscopic analysis. As shown in FIG. 3A, OVA-stimulation increased the proportion of CD4+IL-4+ cells from 4.7% to 9.3%. PG102T or PG102E administration reduced the number by more than 30%. In contrast, CD4+IFN-γ+ cells were slightly increased from 7.2% to 9.2 or 9.6% when animals were treated with PG102T or PG102E, though not in a statistically significant manner. There was little change in the number of both cell types in DEX-administered animals. These results indicated that PG102T and PG102E might regulate the proportion of IL-4- and IFN-γ-producing T cells.

Next, the inventors analyzed the effects of PG102T and PG102E on the IgE-producing B cells. Compared with the DW treatment, oral administration of PG102T or PG102E resulted in a decrease of about 40% in the number of CD19+IgE+B cells. DEX lowered the number of such B cells by more than 60% (FIG. 3B). The confocal microscopic analysis additionally found that there was a significant reduction in the level of IgE production in any given B cell. The signal intensity of IgE in B cells was dramatically increased in OVA-sensitized mice, but it was greatly lowered when animals were treated with PG102T. The effect of PG102E appears to be more potent than PG102T, as the level of fluorescence intensity was further decreased. These results indicated that PG102T and PG102E not only decrease the number of IgE-producing B cells, but also down-regulate the IgE production within a given B cell.

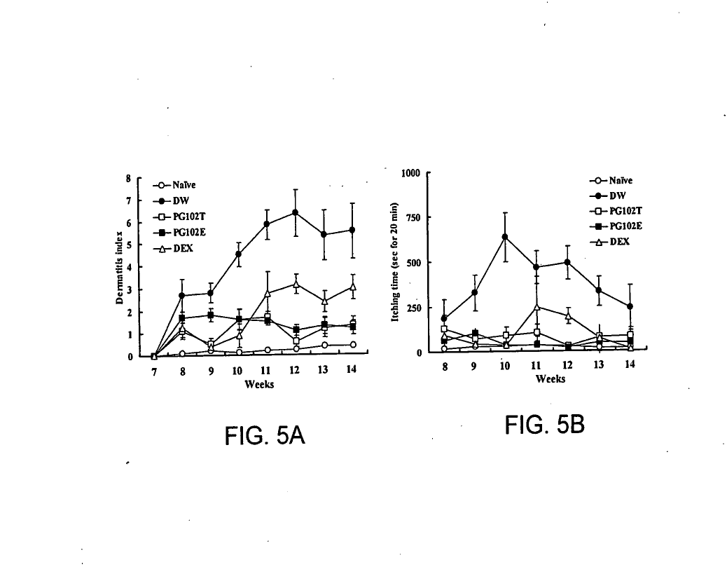

Effects of PG102T and PG102E on cellular transcription factors. To understand the molecular mechanisms underlying the biological activities of PG102T and PG102E, it was investigated whether these two preparations had any effects on cellular transcription factors involved in Th1 and Th2 pathways. This was because the effects of PG102T or PG102E on such multiple cytokines and IgE could more readily be explained if PG102, directly or indirectly, regulates a transcription factor(s). It is well known that transcription factors including GATA-3, T-bet and NFATc2 play major roles in the balancing of Th1/Th2 responses (Lee et al., J Exp Med 2000; 192:105-15; Ting et al., Nature 1996; 384:474-8; Lighvani et al., Proc Natl Acad Sci USA 2001; 98:15137-42; Szabo et al., Cell 2000; 100:655-69; Kiani et al., Blood 2001; 98:1480-8). Splenocytes isolated from OVA-sensitized mice were cultured for 2 days as described above and prepared for immunoblotting analysis. The protein level of GATA-3 was greatly lowered by both PG102T and PG102E, whereas that of T-bet and NFATc2 was upregulated (FIG. 4A). DEX suppressed all these transcription factors tested as previously reported (Adcock, Pulm Pharmacol Ther 2001; 14:211-9).

To verify at what level this regulation occurs, the mRNA levels of respective transcription factors were determined using a quantitative real-time PCR technique (FIG. 4B). PG102T treatment decreased the mRNA level of GATA-3 by almost 3-fold, whereas it increased that of T-bet and NFATc2 by approximately 14-fold and 2-fold, respectively. It is interesting to note that PG102E, which showed higher biological activity in terms of IgE inhibiting effect, changed the levels of GATA3, T-bet and NFATc2 to a lesser extent than PG102T. DEX lowered the levels of both GATA3 and NFATc2, and slightly elevated that of T-bet. These results indicated that PG102T and PG102E might control the production of Th1/Th2 cytokines by regulating the level of transcription factors such as GATA-3 and T-bet at the RNA level.

### Discussion

The biological activities of PG102T and PG102E observed in this study strongly indicate that the extracts from A. arguta are excellent candidates for a potent and unique anti-allergy agent. Both PG102T and PG102E lowered the number of IgE-producing B cells as well as the amount of IgE produced within B cells, ultimately resulting in the decrease of plasma IgE levels. PG102 appears to have such biological activity by modulating the Th1 and Th2 balance through the decrease in the level of selective Th2 cytokines and the increase in that of Th1 cytokines.

The effects of PG102T and PG102E on cellular transcription factors might explain how two preparations work at the molecular level. Both PG102T and PG102E decreased the level of GATA-3, while increasing that of T-bet and NFATc2. GATA-3 is known to strongly trans-activate the IL-5 promoter and the IL-4 enhancer element (Lee et al., J Exp Med 2000; 192:105-15; Ting et al., Nature 1996; 384:474-8). T-bet is involved in the commitment of Th1 cells by inducing the synthesis of IFN-γ in Th1 cells (Lighvani et al., Proc Natl Acad Sci USA 2001; 98:15137-42; Szabo et al., Cell 2000; 100:655-69). In recent studies, it was also reported that T-bet regulates the isotype-switching to IgG2a and the production of IFN-γ in B cells (Gerth et al., Int Immunol 2003; 15:937-44). The present inventors' data are consistent with these known functions of respective transcription factors; namely down-regulation of GATA-3-dependent expression of IL-4 and IL-5, and up-regulation of T-bet-dependent production of IFN-γ and IgG2a in splenocytes. Unlike T-bet and GATA-3, NFATc2 is a non-selectively expressed antigen-inducible transcription factor. Although this protein binds to the enhancer of IL-4 and the IFN-γ promoter in stimulated Th cells, it has been assumed that NFATc2 plays a more important role in driving naïve T cells to effector Th1 cells. Indeed, T cells from NFATc2−/− mice secreted much higher levels of IL-4 than wild type T cells (Kiani et al., Blood 2001; 98:1480-8; Erb et al., Infect Immun. 2003; 71(11):6641-7). The present study showed that PG102T or PG102E increased the level of NFATc2 expression.

The actual molecular mechanism by which PG102T or PG102E favor Th1 responses and suppress IgE biosynthesis is still under investigation. One possibility is that PG102T or PG102E acts on antigen presenting cells, which play a key role in Th cell differentiation through the secretion of soluble factors including IL-12 and the expression of co-stimulatory molecules such as B7-1 (Th1) and B7-2 (Th2) (Kuchroo et al., Cell 1995; 80:707-18). Another possibility is that it works directly on the differentiation process of Th progenitor cells or on the regulation of IgE-producing B cells.

The regulatory effects of PG102T or PG102E on Th1 and Th2 systems may be due to multiple biologically active compounds. To obtain different PG102 preparations in a reliable manner, the present inventors devised the bioassay system based on their effect on the production of IL-4, a major inducer of 1 g isotype switching to IgE, in OVA-stimulated splenocytes. This assay was sensitive and reproducible enough to calculate the specific activity of PG102 and use it to study various factors influencing biological activities of PG102, such as harvest time and different geographic locations for A. arguta. This IL-4-based assay is now being used for the purification of active compound(s) as well as the quality control of PG102 reagents used in a variety of experiments.

The inventors' toxicity data indicate that PG102T is safe. In repeated dose toxicity trials, oral administration of PG102T (500, 1000, 2000 mg/kg/day) for 4-12 weeks produced no adverse effects in rats. These results are in stark contrast with the data obtained from mice administered DEX (0.5 mg/kg/day), which showed severe reductions in body weights and mass of spleens (data not shown). These data, describing the various biological activities of PG102T and PG102E, indicate that extracts of A. arguta are safe, natural preparations, with a minimum risk of side effects, for potential use in the treatment of various allergic disorders.

### Example 2

The following example demonstrates that at least two specific extracts prepared from A. arguta, denoted PG102T and PG102E, have a therapeutic effect on atopic dermatitis.

NC/Nga mice were established as an inbred strain in 1957 based on Japanese fancy mice (Nishiki-Nezumi). When kept under specific pathogen-free (SPF) conditions, mice remain normal and healthy. However, when placed in conventional surroundings, clinical signs begin with scratching behavior initiating from the age of 8 weeks followed by the onset of the eczematous condition. The promptly developed eczema is typically localized on the face, ears, neck and back region. The affected mice display the various clinical signs including hemorrhage, superficial erosion, deep excoriation, scaling, dryness of the skin, and growth retardation (Hiroshi et al., Int Immunol 1997; 9(3):461-466). In the skin lesions, the infiltration of numerous CD4+T cells and eosinophils and the increased number of mast cells with degranulation are observed. In addition, the plasma level of IgE is markedly elevated from the age of 8 weeks, coinciding with the appearance of the skin lesions. The infiltrated cells in the skin lesions express IL-4, IL-5 and TARC, but little or no IFN-γ, resulting in manifestation of Th2-dominant immune reactions (Masayuki et al., Int Ach Allergy Immunol 2003; 132:355-63; Christian et al., Mol Med Today 2000; 5:209-10). These findings resemble the various characters inherent to AD patients, suggesting that a NC/Nga mouse can be an excellent animal model for human AD and also that the modulation of Th1/Th2 and the suppression of IgE biosynthesis may be a therapeutic strategy that can fundamentally improve the clinical symptoms of AD in both humans and mice.

### Materials and Methods

Animals. Specific pathogen free (SPF) female NC/Nga (NC) mice were purchased from SLC (Tokyo, Japan). Mice (5-6 weeks old) were maintained in a SPF environment (SPF NC mice) and provided with autoclaved food and water for at least 1 wk before use. Seven-week-old, SPF NC mice were moved to an air-uncontrolled conventional room (conventional NC mice) Animal experiments complied with the standards set forth in the University Animal Care and Use Committee guidelines at Seoul National University.

Oral administration of PG102T or PG102E. PG102T and PG102E used in this study were prepared from A. arguta as described previously (Park et al., J. Allergy Clin. Immunol., 116:1151-1157, 2005 and Example 1). Conventional NC mice were divided into three groups (n=6-8/group) and were orally treated with PG102T (50 mg/kg/day), PG102E (5 mg/kg/day), DEX (2.5 mg/kg/day) or distilled water (DW; 100 μL/mouse/day) once a day from 7 wks to 14 wks. SPF NC mice, as a negative control, received DW.

Effects of PG102T or PG102E on the development of dermatitis in NC/Nga mice. The severity of dermatitis of each group of mice was assessed once a week from 7 wks to 14 wks of age by two persons blinded to treatment allocations, according to a slight modification of the criteria described by Leung et al. (Leung et al., J Allergy Clin Immunol 1990; 85:927-33). Clinical signs and symptoms seen in conventional NC mice began with itching, erythema and hemorrhage, followed by edema, superficial erosion, deep excoriation, scaling and dryness of skin, and retarded growth. Before skin conditions were scored, scratching behavior was observed for 20 min per mouse and itching index was evaluated by measuring total scratching time for 20 min A total clinical severity score for AD-like lesions was defined as the sum of the individual scores graded as 0 (none), 1 (mild), 2 (moderate) and 3 (severe) for each of five signs and symptoms (itch, erythema/hemorrhage, edema, excoriation/erosion and scaling/dryness).

Measurement of plasma level of immunoglobulins, cytokines, and chemokine. Spontaneously induced allergic responses were monitored by measuring the plasma levels of immunoglobulins including IgE and IgG2a, cytokines, and chemokines. Blood was collected from the retro-orbital plexus with glass capillary tubes at the ages of 7, 10, 12, and 14 weeks, and separated plasma samples were stored at −80° C. until use. The level of IgE was determined by a mouse IgE detection kit (Shibayagi, Gunma, Japan) and that of IgG2a was measured by the sandwich ELISA method as described by Hirano et al. (Hirano et al., J Immunol Methods 1989; 119:145-50). The limit of detection was 1 ng/ml of IgE. The plasma levels of mice IL-4, IL-12, eotaxin and TARC were also measured by ELISA kits (Endogen, Cambridge, Mass. and R&D Systems, Minneapolis, Minn.) according to the manufacturer's instructions.

Th1/Th2 cytokine production by splenocytes derived from NC/Nga mice. At the age of 14 weeks, conventional NC mice orally treated with PG102T, PG102E, DEX or DW and SPF NC mice were sacrificed by decapitation. To study the production of cytokines by splenocytes, spleens of an individual group were obtained, and splenocytes isolated from spleens were resuspended in culture medium (RPMI-1640 containing with 10% heat-inactivated FBS). Splenocyte suspension was seeded to a 24 well culture plate, and the final concentration was adjusted to 5×10⁶ cells/ml/well. These cells were then incubated in the absence or presence of ConA at 2 μg/ml for 3 days. The concentration of ConA was optimized from the preliminary dose-response experiments and no cytotoxicity was found at the concentrations used in this experiment. Following incubation, culture supernatants were collected to determine the level of cytokines (IL-4, IL-5, IL-10, IL-12, IL-13 and IFN-γ) by ELISA as described above.

Analysis of total leukocytes and eosinophils in peripheral blood. At 14 weeks of age, blood was collected from an individual group of mice. The number of total leukocytes and eosinophils in the heparinized blood was counted using a Celldyn hemocytometer (Abbott; Santa Clara, Calif.).

Histological analysis and measurement of epidermal and dermal thickness in AD-like skin lesions. For histological examination, small biopsies were obtained from the head, neck and dorsal skin of conventional and SPF NC mice at the age of 14 wks. Skin sections were fixed in 10% phosphate-buffered formalin (pH 7.2), embedded in paraffin, cut at 4 μM, and stained with H&E for detecting various inflammatory cells. Cells between epithelium and panniculus carrnosus were observed under a microscope at a magnification of 400×. After the microscopic fields were photographed, the thickness of both the epidermis and dermis were measured as the distance from the stratum corneum of the epidermis to the basement membrane of the dermis. The distance was expressed as the mean of three random fields for which 5 measurements were averaged.

Determination of cytokine and chemokine expression in the skin biopsies. The levels of IL-4, IL-5, eotaxin and TARC in the skin biopsies from the face were measured by ELISA. Briefly, the tissue from facial skin lesions was excised, homogenized in lysis buffer, and then the freezing/thawing procedure was repeated three times. After centrifugation, the supernatants containing total cellular protein were quantified and used to detect the level of cytokines and chemokines. Results were normalized to the total amount of protein prepared from tissue lysates. The protein lysate samples prepared from the facial skin tissue as described above, were also subjected to Western blotting using antibodies specific to mice GATA-3, pSTAT6 (Santa Cruz Biotechnology, Santa Cruz, Calif.) or β-actin (Sigma) as a loading control.

Statistics. Data were expressed as mean±SEM, and differences between mean values were analyzed by unpaired Student's t test. P values less than 0.05 or 0.01, which were calculated as one-tailed P values, were considered statistically significant.

### Results

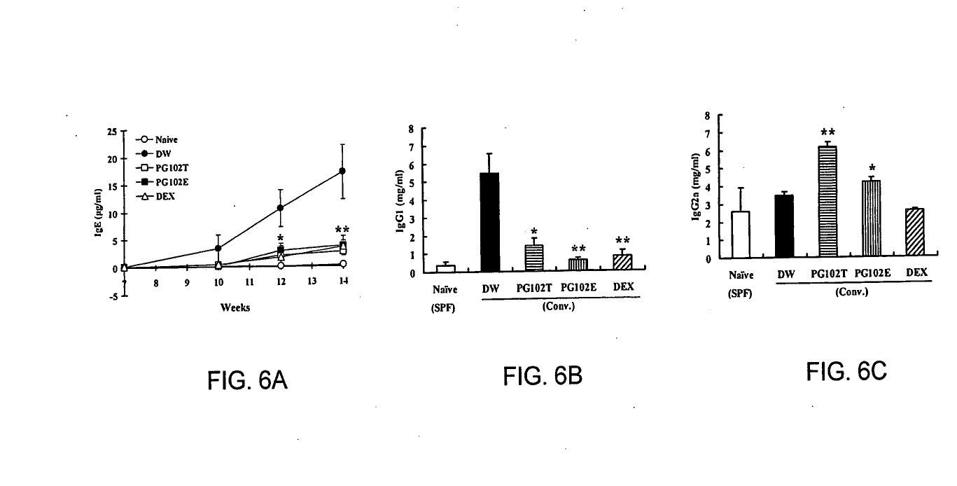

Oral administration of PG102T and PG102E inhibits the development of spontaneous dermatitis in conventional NC mice. To examine the possible effects of PG102 on atopic dermatitis, the inventors used NC mice as a model for human atopic dermatitis, which show atopic dermatitis-like skin lesions with aging under conventional conditions. Conventional NC mice were orally administered PG102T [50 mg (60 units)/kg/day], PG102E [5 mg (54.5 units)/kg/day], DEX (2.5 mg/kg/day) or DW (100 μL/mouse/day) on a daily basis for 7 weeks and the progression of atopic dermatitis was observed. The dosage of PG102T and PG102E was based on the concentration that gave therapeutic effects in the OVA-sensitized murine model used in the previous experiments (see Example 1 and Park et al., J. Allergy Clin. Immunol., 116:1151-1157, 2005). The increase in the dermatitis severity scores of conventional NC mice treated with DW showed that the development of dermatitis progressed in an age-dependent manner (FIG. 5A). However, oral administration of PG102T or PG102E significantly decreased the score from 9 weeks of age. The improvement of dermatitis severity was accompanied by a reduction in the scratching incidence. Treatment with PG102T greatly lowered the scratching time from 9 weeks of age. Similar results were observed in animals treated with PG102E (FIG. 5B). Moreover, these outcomes were consistent with the observation made through the analysis of overall clinical visual features of the mice (data not shown). DEX also decreased the dermatitis severity and scratching behavior. These data indicated that PG102T and PG102E might suppress the spontaneously induced dermatitis in this animal model.

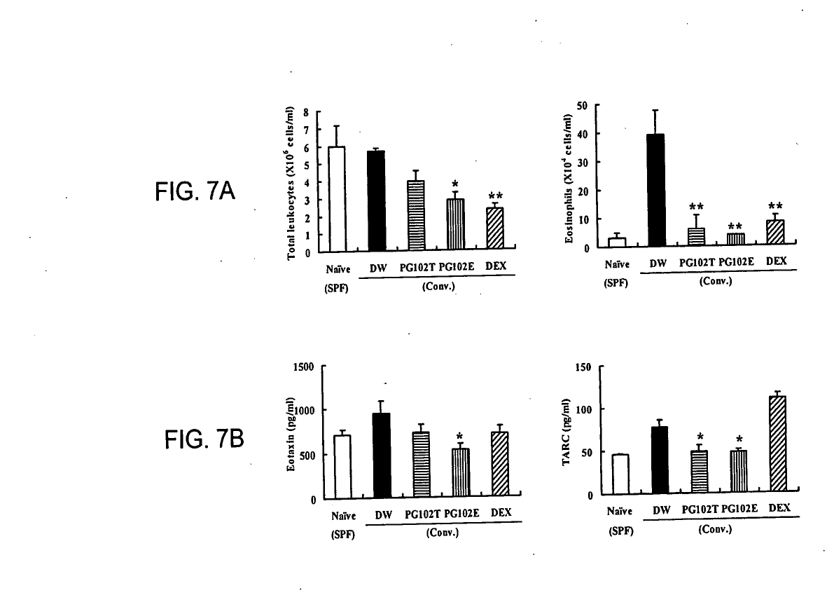

PG102T and PG102E decrease the production of IgE and IgG1, while they increase that of IgG2a in plasma. In addition to the visual clinical features imitating human atopic dermatitis, conventional NC mice also show the elevated level of IgE in plasma after the onset of dermatitis. Therefore, it was tested whether oral administration of PG102T or PG102E could control the plasma level of Th2-mediated IgE and IgG1 and Th1-mediated IgG2a. From 7 weeks of age, animals were orally fed with PG102T, PG102E, DEX or DW on a daily basis, and blood samples were obtained at the age of 7, 10, 12 and 14 weeks. Under SPF conditions, NC mice normally produced approximately 150 ng/ml of total IgE, but when animals were placed under conventional conditions, IgE levels gradually increased with aging, to almost 17 μg/ml at 14 weeks of age. Administration of PG102T or PG102E lessened the plasma level of IgE from 10 weeks of age in a statistically significant manner, resulting in a 5-fold lower level of IgE at the end of the experiment. The IgE-lowering effect of PG102T and PG102E was comparable to that of DEX used as a positive control (FIG. 6A). At 12 weeks of age, the level of IgG1, another Th2-mediated Ig class, was measured. Under conventional conditions, DW-treated mice produced a level of IgG1 greater than 5 mg/ml. However, administration of PG102T and PG102E decreased its level by 75% and 90%, respectively. The level of IgG2a, which belongs to the Th1-mediated Ig class, was increased by approximately 180% in conventional NC mice treated with PG102T. PG102E also induced the level of IgG2a in plasma (FIG. 6B). These data indicated that PG102T and PG102E might suppress the development of dermatitis by lowering the levels of IgE and IgG1 and by increasing that of IgG2a.

PG102T or PG102E may regulate the balance of Th1/Th2 cytokine production in plasma and splenocytes. The above data indicated that PG102T and PG102E affects the expression of Th1 and Th2 cytokines. To understand the activity of PG102T and PG102E on a molecular and cellular level, the levels of IL-4 and IL-12, representing the Th2 and Th1 pathways, respectively, were measured in plasma at 12 weeks of age. Compared with conventional NC mice treated with DW, the level of IL-4 was lowered in mice treated with PG102T or PG102E by 60% and 76%, respectively (Table IVA). Oral administration of PG102T or PG102E elevated the level of IL-12 in a statistically significant manner.

The inventors also analyzed the effects of PG102T and PG102E on the production of Th1 and Th2 cytokines in splenocytes isolated from NC mice. Mice were sacrificed at 14 weeks of age and spleens were obtained to isolate the splenocytes. Splenocytes from each group of mice were stimulated with a T cell-specific mitogen, ConA, for 3 days, and the levels of various cytokines were detected. In the presence of ConA, the level of all Th2 cytokines was highly elevated, but treatment with PG102T or PG102E reduced the levels of IL-4, IL-5 and IL-10 by 24% to 78% (Table 1VB). DEX also inhibited the levels of all three Th2 cytokines, although the decrease of IL-5 by DEX was not statistically significant. The level of IL-13 was not influenced by PG102 or DEX.

TABLE IV
Effects of PG102T and PG102E on the level of Th1 and Th2 cytokines in plasma and cultures splenocytes
A. Plasma levels

| Sources Conven- | | | | |
|---|---|---|---|---|
| tional NC mice | IL-4 | | IL-12 | |
| DW | 474 ± 31 | (100) | 859 ± 17 | (100) |
| PG102T | 189 ± 64 | (40)** | 1217 ± 140 | (142)* |
| PG102E | 113 ± 69 | (24)** | 1106 ± 77 | (129)* |
| DEX | 80 ± 56 | (17)** | 236 ± 140 | (27) |
| SPF NC mice | 13 ± 5 | (3) | 1708 ± 116 | (199) |

B. Splenic levels

Sources Conven-

| tional NC mice | IL-4 | | IL-5 | | IL-10 | | IL-13 | | IL-12 | | IFN-γ | |
|---|---|---|---|---|---|---|---|---|---|---|---|---|
| DW | 134 ± 17 | (100) | 680 ± 98 | (100) | 782 ± 21 | (100) | 448 ± 1 | (100) | 656 ± 71 | (100) | 10407 ± 160 | (100) |
| PG102T | 30 ± 6 | (22)** | 357 ± 52 | (53)* | 621 ± 1 | (79)* | 348 ± 7 | (78) | 957 ± 68 | (146)* | 15848 ± 2074 | (152)* |
| PG102E | 54 ± 7 | (40)** | 203 ± 6 | (30)** | 594 ± 19 | (76)* | 406 ± 11 | (91) | 1636 ± 42 | (249)** | 18050 ± 1380 | (173)** |
| DEX | 54 ± 11 | (40)** | 531 ± 283 | (78) | 416 ± 30 | (53)** | 417 ± 2 | (93) | 149 ± 22 | (23) | 10096 ± 236 | (97) |
| SPF NC mice | 12 ± 2 | (9) | 33 ± 8 | (5) | 37 ± 3 | (5) | ND | | 166 ± 1 | (25) | 390 ± 88 | (4) |

Plasma samples were isolated from each group of mice at the age of 12 weeks. All splenocytes from NC mice were stimulated with ConA during the culture. Values are expressed as means ± SEM for five animals.
*P < 0.05 and
**P < 0.01, vs DW-treated mice (Student's T-test).
ND, not detectable.

When cells from conventional NC mice were grown in the presence of ConA, the levels of IL-12 and IFN-γ were augmented. In SPF NC mice, the level of IL-12 was 166 pg/ml, but the stimulation with ConA increased that by 4-fold. Treatment with PG102T or PG102E further induced the level of IL-12 by approximately 150% or 250%, respectively. The level of IFN-γ was also dramatically increased in the presence of ConA to almost 11 ng/ml. PG102T administration elevated the level of this cytokine by approximately 150%. PG102E appeared to be more potent than PG102T. DEX potently suppressed the level of IL-12, while it did not affect that of IFN-γ. In summary, PG102T and PG102E increased the level of Th1 cytokines, while decreasing that of selective Th2 cytokines, unlike DEX, which indiscriminately inhibited the expression of almost all cytokines measured in this study.

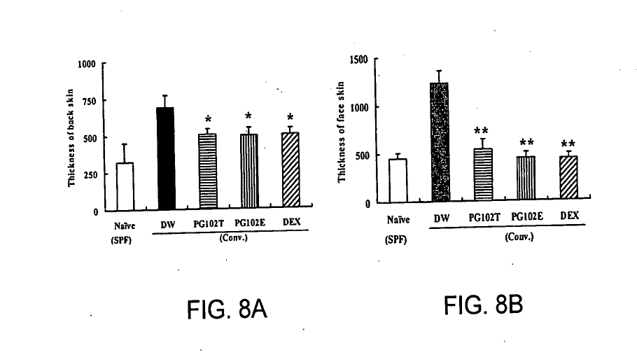

PG102 not only prevents eosinophilia, but also decreases the level of eotaxin and TARC. Dermal infiltration of inflammatory cells including eosinophils is an important feature of atopic dermatitis in NC mice. Because the presence of inflammatory cells in the skin lesions may have resulted from their mobilization from bone marrow into blood, the inventors first analyzed the number of total leukocytes and eosinophils in the peripheral blood at 12 weeks of age. As shown in FIG. 7A, the number of total leukocytes of conventional NC mice was increased upon the onset of the dermatitis. In particular, the number of eosinophils was greatly increased in DW-treated mice under conventional conditions, resulting in eosinophilia. However, PG102T or PG102E administration lowered the number of both total leukocytes and eosinophils, presumably contributing to the prevention of eosinophilia.

The changes in the number of circulating eosinophils may be manifested by the production of chemokines, leading to chemoattraction in response to inflammation (3-6). Therefore, the plasma levels of eotaxin and TARC, which are representative chemoattractants for eosinophils and Th2 cells, were determined. In the conventional environment, DW-treated mice produced an increased level of eotaxin and TARC, but these levels were decreased in mice treated with PG102T or PG102E by approximately 25% to 50% (FIG. 7B). There was little change in the animals administered DEX. These results showed that PG102T and PG102E inhibited the production of eotaxin and TARC, resulting in the prevention of Th2-mediated eosinophilia, which generally coincides with the onset of dermatitis in NC mice.

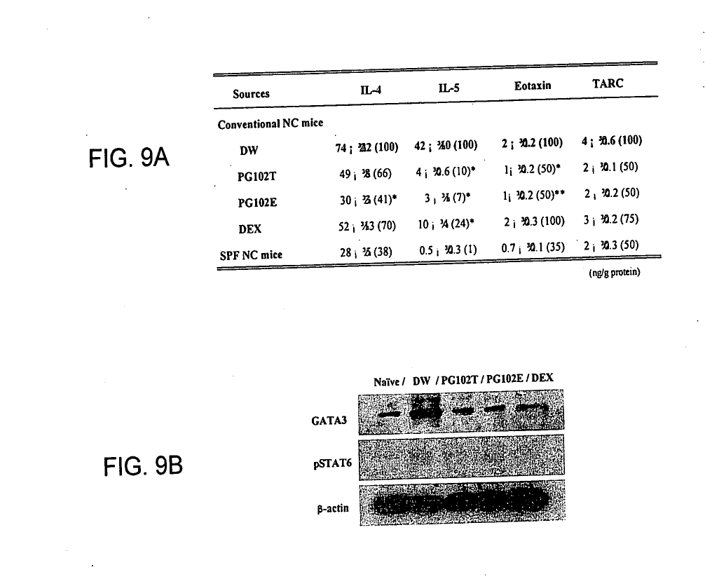

PG102T or PG102E administration inhibits the infiltration of inflammatory cells in dermis and the thickening of the epidermis and dermis. Improvement of clinical skin condition and inhibition of Th2 response by PG102T and PG102E was also confirmed by the analysis of H&E stained sections at 14 weeks of age. Mice fed with DW exhibited a marked thickening of the epidermis and dermis, prominent hyperkeratosis, infiltration of inflammatory cells, and hemorrhage. Morphologic study indicated that these infiltrating cells in the dermis were eosinophils, mast cells and lymphocytes. However, treatment with PG102T or PG102E for 7 weeks inhibited the thickening of the epidermis and dermis and the infiltration of inflammatory cells in the dermis, resulting in the histological environment very similar to that of SPF NC mice (FIG. 8A). Administration of DEX also produced similar results, but greatly expanded the adipocyte region. Measurement of epidermal and dermal thickness in face and back skin also demonstrated that both PG102T and PG102E prevented hyperplasia of the epidermis and dermis in a statistically significant manner (FIG. 8B). These results indicated that oral intake of PG102T or PG102E could effectively suppress the development of dermatitis in NC mice with little or no side effects.

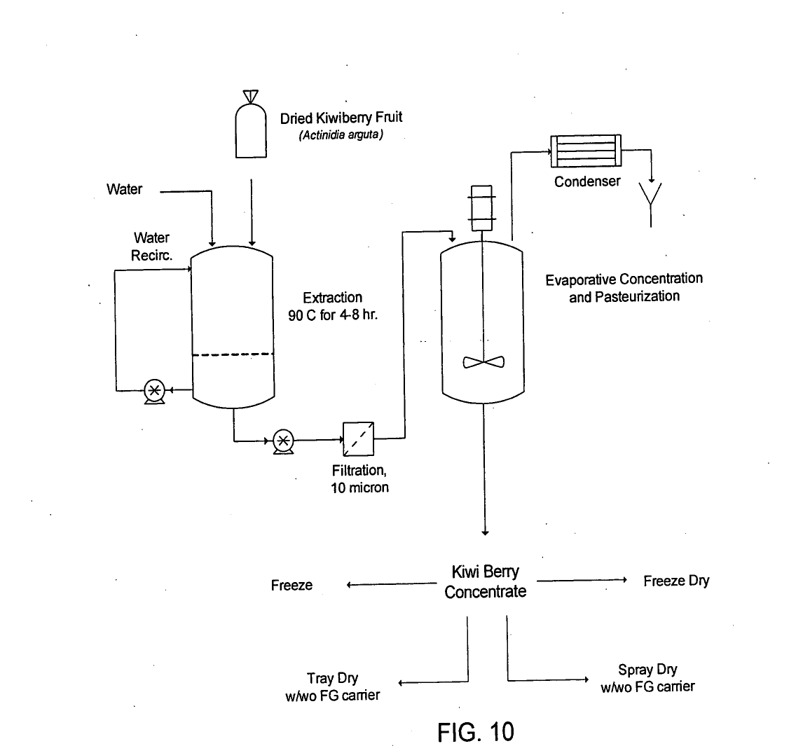

PG102 or PG102E reduces the expression of Th2-mediated cytokines and chemokines through the down-regulation of GATA3. To investigate the effects of PG102T or PG102E on the Th2-mediated production of cytokines and chemokines in the skin of mice, the levels of IL-4, IL-5, eotaxin and TARC were measured by ELISA at 14 weeks of age. In the skin from SPF NC mice, all four proteins were weakly expressed, but their levels were notably increased in conventional NC mice. Administration of PG102T or PG102E lowered the levels of IL-4, eotaxin and TARC by more than 30%. The effect on IL-5 expression was more prominent, with its level being increased by almost 90%. On the contrary, DEX inhibited the levels of IL-5 and TARC, but not that of IL-4 and eotaxin (FIG. 9A).

Based on these findings, the expression of transcription factors including STAT6 and GATA3 was determined by immunoblotting. STAT6 and GATA3 are well known to play critical roles in the differentiation of Th2 cells and the production of Th2-specific cytokines and chemokines (Arakawa et al., Clin Exp Immunol 2004; 135(3):505-10; Gunther et al., J Allergy Clin Immunol 2004; 113:987-94; Konishi et al., Proc Natl Acad Sci 2002; 99(17):11340-5). As shown in FIG. 9B, the level of GATA-3 protein was lowered by both PG102T and PG102E. The expression of phosphorylated STAT6 (pSTAT6) was also decreased in the conventional NC mice treated with PG102T, while it might not be the case with PG102E. DEX suppressed the level of both GATA3 and pSTAT6. These results demonstrated that PG102T and PG102E might down-regulate the level of Th2-specific cytokines and chemokines by inhibiting the expression of GATA3.

### Discussion

AD is a major allergic disease that often begins during infancy. A significant fraction of affected individuals develop asthma and/or allergic rhinitis later in life (Leung, Clin Exp Immunol 1997; 107(suppl. 1):25-30). AD results from dermal inflammation caused by an abnormal immune response, in particular, overactivation of the Th2 pathway. The inventors have discovered that PG102T and PG102E, water-soluble fractions derived from A. arguta, control the production of selective Th1- and Th2-mediated cytokines and also that of IgE in the OVA-sensitized mouse model (see Example 1). Based on these data, it was reasoned that these plant extracts might be useful for the treatment of various allergic diseases (Mayaumi et al., J Allergy Clin Immunol 2000; 106:159-66; Hisae et al., Phytother Res 2001; 15:506-10). In this example, the inventors investigated whether PG102 could produce any actual therapeutic effects(s) on atopic dermatitis using NC/Nga mice as a model system. The results indicated that PG102T and PG102E suppressed the development of spontaneously induced dermatitis, which, without being bound by theory, is believed to be through the control of various Th1- and Th2-associated factors, namely the downregulation of IL-4, IL-5, IL-10, and IgE, as well as the upregulation of IL-12 and IFN-γ. The biological consequences of such biochemical changes in the NC/Nga mouse model include the highly decreased number of eosinophils in the peripheral blood as well as in the skin lesions, the suppression of the thickening of the epidermis and dermis, and the inhibition of the infiltration of various inflammatory cells. Of particular interest is a decrease in the regional expression levels of eotaxin, TARC, IL-4 and IL-5. The levels of these proteins are abnormally high in the skin lesions of NC/Nga mice grown under the conventional environment. When animals were orally administered PG102, however, these chemokines and cytokines were found to be at the virtually normal level. Eotaxin, together with IL-5, is known to be a potent chemoattractant for eosinophils, while TARC produced by keratinocytes (and also Th2 cells) is thought to attract Th2 cells, and induces the pathological responses typically associated with atopic dermatitis. In this regard, it is worth noting that the receptors for eotaxin and TARC are CCR3 and CCR4, respectively, which are both highly expressed in Th2 cells (Christian et al., J Clin Invest 1999; 104:1097-105; Tomomi et al., J Allergy Clin Immunol 2110; 107:353-8; Weilie et al., J Clin Invest 2002; 109:621-8; Masayuki et al., Int Ach Allergy Immunol 2003; 132:355-63).

The detailed molecular mechanism(s) and the exact sequence underlying the therapeutic effects of PG102, observed in the NC/Nga mouse model, are still being investigated. It is possible that PG102 acts first on cellular transcription factors, for example GATA-3, and subsequently on the expression of key cytokine players involved in the Th1 and Th2 systems such as IL-4 and IFN-γ, inducing the cascade reaction, leading to a decrease in the level of IgE and respective chemokines (Zhu et al., Nat. Immunol. 2004; 11:1157-65). Alternatively, PG102 may initially work at the local level; for example, decreasing the level of eotaxin and TARC, inhibiting their chemoattractive functions, lowering the number of eosinophils at the systemic and local levels, and suppressing the histopathological development seen in conventionally grown NC/Nga mice. The activity of PG102 may be due to multiple compounds acting at various levels of allergy-related biochemical pathways. Overall, PG102 appears to operate on key biological factors related to atopic dermatitis in this animal model, treating the condition at its source rather than simply providing relief of symptoms.

PG102T and PG102E were derived from an edible fruit. No toxicity was found in repeated dose toxicity experiments, in which 2000 mg/kg/day, 40-fold higher than the concentration used in this study, was administered on a daily basis for 12 weeks. Together with data from the previous experiments involving OVA-sensitized mice, the results from NC/Nga mice demonstrate that PG102 is a safe and effective reagent for the treatment of various allergic diseases including atopic dermatitis. Given that the prevalence of atopic diseases is increasing in all major developed countries and virtually no reagent is available for the fundamental treatment of atopic dermatitis, preparations such as those described herein represent advances in the field.

### Example 3

The following example shows the preparation of various preparations comprising A. arguta that were used in the examples below.

### Plant Material

Stems (consisting of canes and fruiting spurs), roots, and bark of Actinidia arguta (Sieb. Et Zucc.) Planch. ex Miq. (Actinidaceae) cultivar ‘Ananasnaya’ were collected at Hurst Berry Farm, Sheridan, Oreg. A voucher specimen (#518640) was authenticated by Mr. Tim Hogan, Collection Manager, University of Colorado Herbarium, The University of Colorado, Boulder, Colo., and deposited at the same location. Plant material was air dried 48 hours and stored at room temperature prior to extraction or other processing. Ripe, ready-to-eat A. arguta fruit were collected at Hurst Berry Farm, frozen immediately, shipped and stored frozen (−20° C.) prior to extraction or other processing.

### Extracts and Other Preparations

Powdered stems (126.6 g), powdered roots (79.0 g), and finely divided bark (126.2 g) were each extracted with distilled water (1 L) at 94° C. for 4 h. The mixtures were then filtered, and the filtrate concentrated to dryness by rotary evaporation to provide a stem extract (9.9 g), a root extract (8.6 g), and a bark extract (2.4 g).

Twenty frozen A. arguta berries (154.4 g) were thawed at room temperature, crushed, and extracted with distilled water (1 L) 91° C. for 5 h. The mixture was filtered, and the filtrate concentrated to produce a ‘boiled’ fresh fruit extract (12.8 g).

Additional fresh-frozen kiwifruit (341.6 g) was thawed and run through a juicer. The juicer removed the skins from the fruit resulting in a mixture of seeds, pulp, and juice. This mixture was centrifuged (30 min, ˜3500 rpm) to provide 150 mL of juice. This juice was concentrated to dryness by rotary evaporation resulting in a fruit juice concentrate (24.2 g).

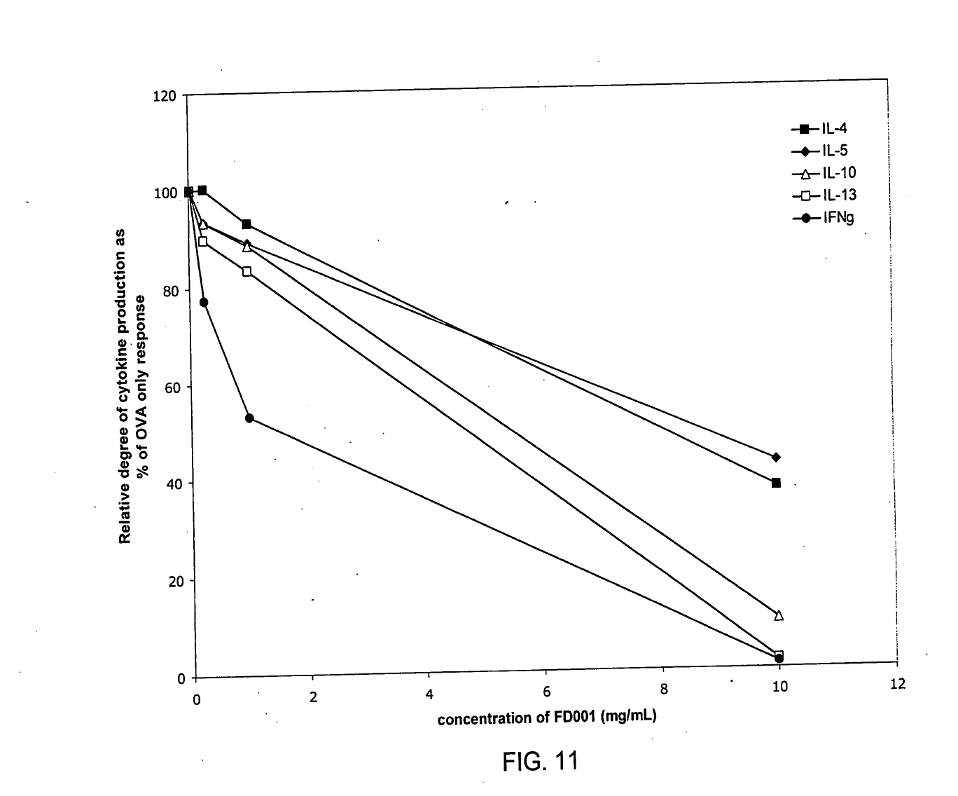

In order to generate larger quantities of an extract equivalent to PG102T (as described in Example 1), a process scale extraction of the kiwifruit was performed (Sungil Bioex Co., Ltd., Bibong, Korea). Frozen kiwifruit (1242 kg) were sliced (¼″ to ⅜″ thickness) and dried in a convection dryer (65-80° C.) to a moisture content of 5-20%. Batch extraction (FIG. 10) of the dried fruit (239 kg) was performed in a jacketed stainless steel reactor with an internal filter screen to support the extraction load. An external condenser was employed to prevent water loss during the extraction. The quantity of extraction solvent (water) was based on 5-10 times the weight of the dried fruit to be extracted. The contents of the extraction vessel were heated from 0 to 90° C. over a period of 2 h via the introduction of steam into the jacket of the reactor. Water (90° C.) was then recirculated through the biomass using an external recirculation loop for 4-12 h. Subsequently, the spent biomass was removed for disposal and the aqueous extract filtered through a 10 micron filter. The filtrate was then concentrated under vacuum (˜600 mmHg) at 55-65° C. in an agitated stainless steel reactor equipped with an external condenser and a distillate receiver. Once the material was concentrated, it was held at 80° C. for an additional 30 min to sterilize the extract. The resulting material (101 kg), equivalent to PG102T, was designated FD001. Of this material, 3 kg were set aside for further testing. Good Manufacturing Practices were used throughout the process.

To create a powdered material appropriate for encapsulation and useful in the clinical applications described herein, the FD001 concentrate produced above (98 kg) was pumped to a horizontal paddle blender and mixed with an equal weight, based on the calculated solids content, of microcrystalline cellulose (MCC). Following this, the solid blend was transferred to stainless steel trays that were placed into a forced hot air dryer (70-80° C.) for 24 h. The dry, lumpy solids were then ground in a Fitzmill type hammer mill to produce a 40 mesh powder (118 kg). This material was encapsulated (GMP Laboratories of America, Inc., Anaheim, Calif.) into 300 mg- or 600 mg-sized capsules, each containing a 1:1 mixture of FD001 and MCC for use in canine and human clinical trials.

Dried A. arguta fruit (7.0 g) from process-scale material, sliced and dried as in the initial steps above (but not subjected to the batch extraction), was powdered and this material was extracted with water (250 mL) 25° C. for 4 hours. The mixture was filtered and the filtrate concentrated to dryness by rotary evaporation to provide a room temperature water extract of the dried fruit (4.2 g).

FD001 (79.9 g) was blended with 1.5 L distilled H₂O and this solution extracted successively with four 500 mL portions of ethyl acetate (EtOAc). The combined organic layers and the aqueous layer were concentrated to dryness in vacuo resulting in an EtOAc extract (7.4 g) and the aqueous remainder (41.5 g).

### Example 4

The following example describes in vitro testing for immunomodulating activity in A. arguta preparations.

The purpose of this study was to compare the relative ability of various extracts and preparations produced from A. arguta to modulate cytokine production (IL-4, IL-5, IL-10, IL-13, and IFNγ) in splenocyte cultures derived from ovalbumin (OVA, grade V, Sigma)-sensitized mice using ELISA (Quantikine kits, R&D systems) analysis. The following samples (prepared as described in Example 3 above) were tested: FD001 (PG102T), the fruit juice concentrate, and the EtOAc extract.

### Splenocyte Isolation and Culturing

Female, Balb/c mice (Harlan, Indianapolis, Ind.) were sensitized by IP injection of 20 μg OVA on days 0 and 14. On day 24, following euthanasia by cervical dislocation, spleens were aseptically removed from individual mice and immediately processed for splenocyte culture development using sterile technique. The spleens were dissociated in the presence of 10 mM HEPES-buffered RPMI-1640, by gently forcing the tissues through the grid of a 70 micron nylon mesh using the plunger from a 3 cc syringe. Large cell aggregates were removed from the resulting suspension using a FCS-gradient. The splenocytes were then centrifuged (1500 rpm, 5 min) and the resulting cell pellets were treated with RBC lysis buffer (10 mM, RT) to remove the contaminating erythrocytes. The majority of the RBC lysis buffer was then removed by centrifugation (1500 rpm, 5 min) and the pelleted splenocytes were then washed 3× with 10 mM HEPES-buffered RPMI-1640. Following the final wash, the pelleted splenocytes were resuspended in a volume of in RPMI-1640 containing 10% FCS and Penn/Strep (complete medium) designed to deliver a final cell density of 5×10⁶ cells/mL. For each analysis, 5×10⁶ splenocytes were plated into the individual wells of a 24-well plate. On day 3, the supernatants from these wells were collected and frozen in preparation for the determination of experimental results.

Control splenocyte cultures were also established from naïve (non-sensitized) mice in the manner described above and plated out into the individual wells of a 24-well plate to achieve a final cell density of 5×10⁶ cells/mL. These splenocytes were established in RPMI-1640 containing 10% Fetal Calf Serum, Penn/Strep, and they received no additional treatment. On day 3, the supernatants from these wells were collected and frozen to serve as negative experimental controls.

Stimulation of Splenocyte Cultures with A. Arguta Preparations

Ten OVA-sensitized mice were used for each preparation tested. Splenocytes from each mouse were plated (5×10⁶ cells/mL) into 8 individual wells of a 24-well plate in complete RPMI-1640 medium containing 100 μg/mL OVA, 0.5% DMSO and either no or chosen concentrations of each of the specific test preparations under examination. 6 of the 8 wells were partitioned into 2 sets of 3 wells. Each of the wells in the sets of 3 were treated with A. arguta preparations at concentrations of either 0.25, 1.0 or 10 mg/mL. To serve as positive controls, the 7^{th} wells were treated with 2 μM dexamethasone (DEX), a potent glucocorticoid anti-inflammatory. The 8^{th} wells received no additional treatment and served as an OVA-only experimental control. After 3 days of culture, the supernatants from each of the unique 8 wells per OVA-sensitized mouse were collected and frozen. These supernatants were used to determine the levels of the cytokines IL-4, IL-5, IL-10, IL-13, and IFN-γ present in the culture medium.

### Determination of Cytokine Levels in Culture Supernatants

The cytokine levels in the culture supernatants derived from the A. arguta preparation-treated splenocytes, DEX-treated splenocytes, OVA-only treated splenocytes, and untreated splenocytes from non-sensitized control mice were determined by ELISA assay. Two replicate ELISA plate wells were utilized for each cytokine level determination.

### Results

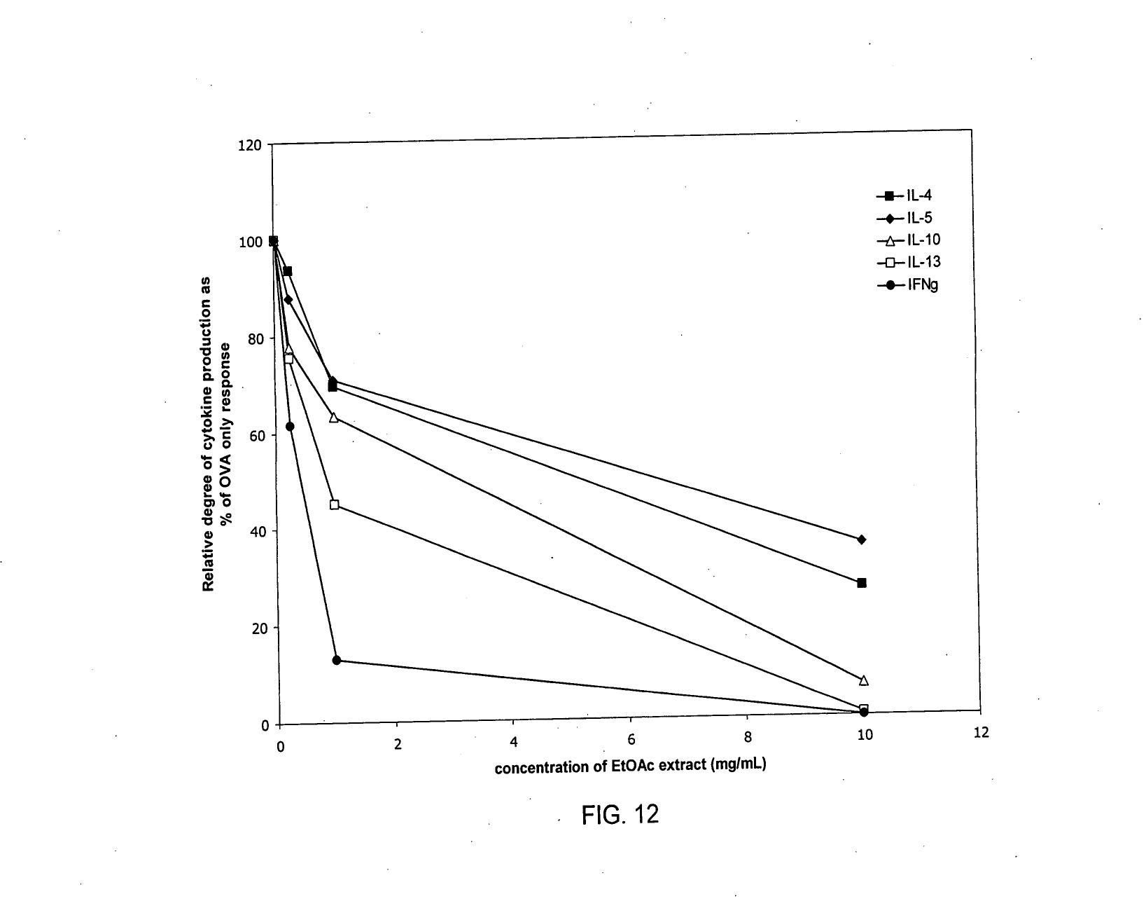

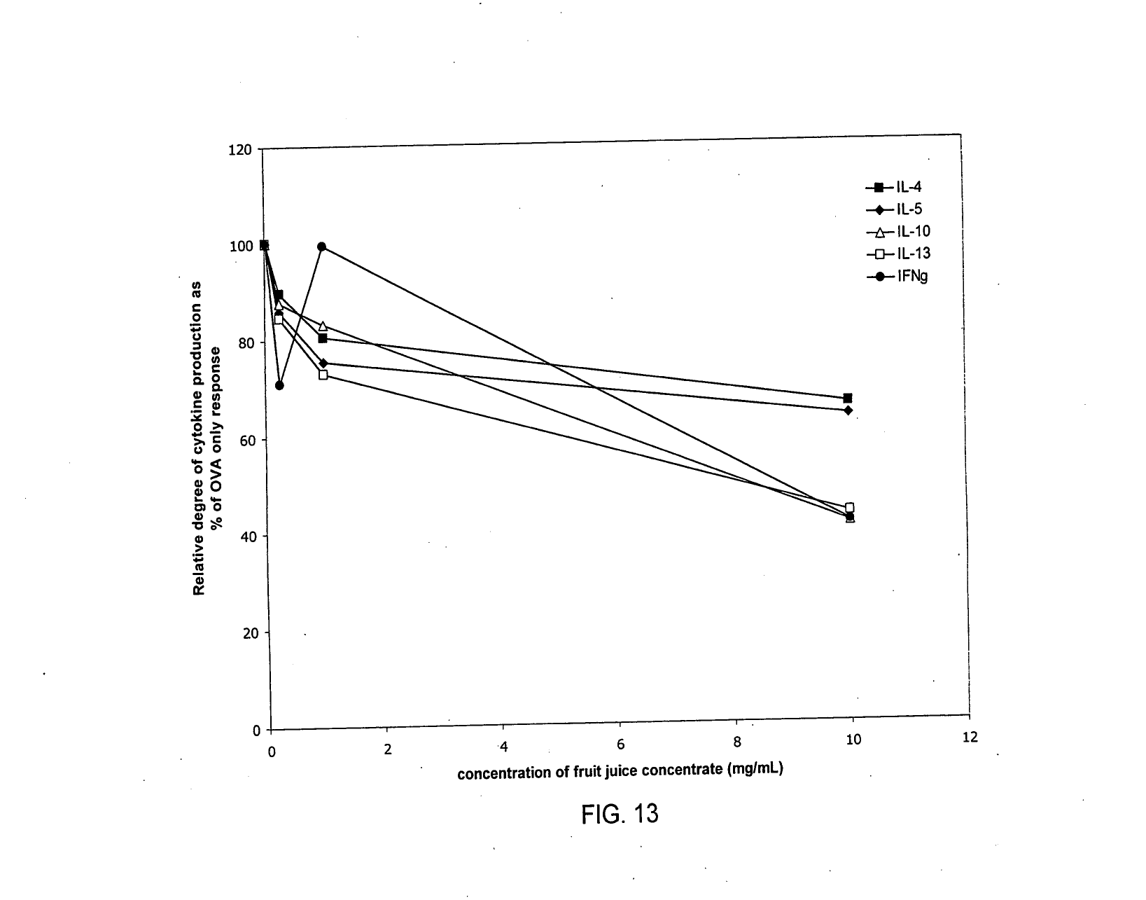

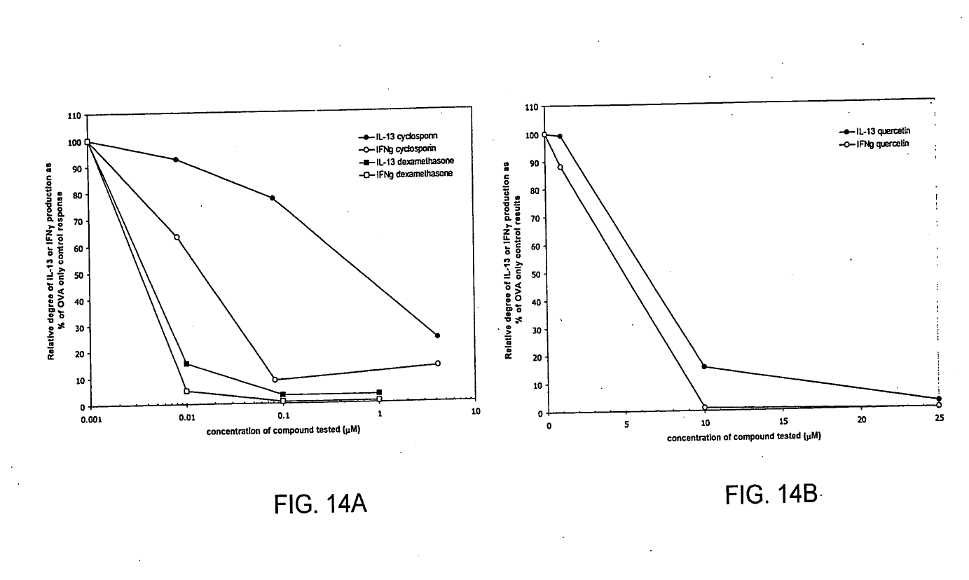

This in vitro work confirmed activity in the PG102T-equivalent FD001 (FIG. 11), the EtOAc extract (FIG. 12), and the fruit juice concentrate (FIG. 13) of A. arguta. It was observed that all three preparations (at 10 mg/mL) caused substantial suppression, to varying degrees, of the cytokines IL-4, IL-5, IL-10, IL-13, and IFNγ. The most pronounced effects were seen on IL-13 and IFN-γ for all of the samples examined, consistent with prior in vitro work (Table I; Example 1). Since activity was observed in the EtOAc extract, it is evident that active constituents present in FD001 are extractable into organic solvents, and may be further purified by traditional chromatographic methods. Significantly, the fruit juice concentrate also suppressed cytokine production by the splenocytes, indicating that extraction of the kiwifruit as shown in Examples 1 and 2 is not the sole requirement to produce active preparations of hardy kiwifruit. It is noted that relatively less suppression of cytokines was apparent in the fruit juice concentrate, suggesting that the drying or heating process used to prepare FD001 may be important for enhancement of activity.

### Example 5

The following example describes a comparison of in vitro activity of extracts of non-fruit parts of A. arguta, as well as alternative fruit preparations of A. arguta.

The purpose of this study was to assess the ability of A. arguta extracts that originate from plant parts other than the fruit, or from alternative preparations of the fruit (i.e., other than the extracts described in Examples 1 and 2), to modulate cytokine production (IL-13 and IFNγ) in splenocyte cultures derived from ovalbumin-sensitized mice, using ELISA analysis. The following samples (prepared as described above) were tested: water extracts of the stem, root, bark of A. arguta, prepared as described in Example 3; “boiled” fresh fruit preparations; the fruit juice concentrate prepared as described in Example 3; FD001 (large scale equivalent of PG102T) prepared as described in Example 3; FD001 powder prepared as described in Example 3 (used for clinical trials described below); a room temperature water extract of the dried A. arguta fruit; the EtOAc extract prepared as described in Example 3; and aqueous remainder, also described in Example 3. In addition, the activity of three known immunosuppressive compounds, cyclosporin, dexamethasone, and quercetin were evaluated as controls.

### Splenocyte Isolation and Culturing

Preparation of the splenocytes was performed in a manner identical to that described above for Example 4.

Stimulation of Splenocyte Cultures with A. Arguta Extracts

Splenocyte cells from 8 OVA-sensitized mice (8 replicates) were utilized for the analysis of each extract or preparation tested. 5×10⁶ cells splenocyte cells from each mouse were plated out into the individual wells of a 24-well plate in complete RPMI-1640 medium containing 100 μg/mL OVA and 25 mM HEPES (pH 7.3), 1 ml per well. Kiwifruit preparations were examined at concentrations of 1.0, 3.0, and 10 mg/mL.

### Cyclosporin, Quercetin, and Dexamethasone Analysis

Splenocytes from 8 OVA-sensitized mice (8 replicates) were utilized for the analysis of each compound tested, with the exception of Quercetin where the splenocytes derived from only 2 OVA-sensitized mice were examined. 5×10⁶ cells splenocytes from each mouse were plated out into the individual wells of a 24-well plate in complete RPMI-1640 medium containing 100 μg/mL OVA and 25 mM HEPES (pH 7.3), 1 ml per well. Cyclosporin was examined at concentrations of 0.0083, 0.083, and 4.15 μM. Dexamethasone was examined at concentrations of 0.01, 0.1 and 1 μM. Quercetin was examined at concentrations of 1, 10, and 25 μM. Wells treated with 1 μM dexamethasone, a potent glucocorticoid anti-inflammatory, served as positive experimental controls. Wells receiving only complete RPMI-1640 containing 100 μg/ml OVA and 25 mM HEPES (pH 7.3) served as OVA-only experimental controls. After 3 days of culture, the supernatants were collected and frozen. These supernatants were used to determine the levels of IL-13, and IFN-γ present in the various culture media, under the experimental conditions examined.

### Determination of Cytokine Levels in Culture Supernatants

The cytokine levels in the culture supernatants from all treatment and control wells were determined by ELISA assay. Two replicate ELISA plate wells were utilized for each cytokine level determination.

Based on the results of the in vitro testing described in Example 4, only the expression of IL-13 and IFN-γ were analyzed for the purpose of estimating levels of activity present in the materials tested.

### Results

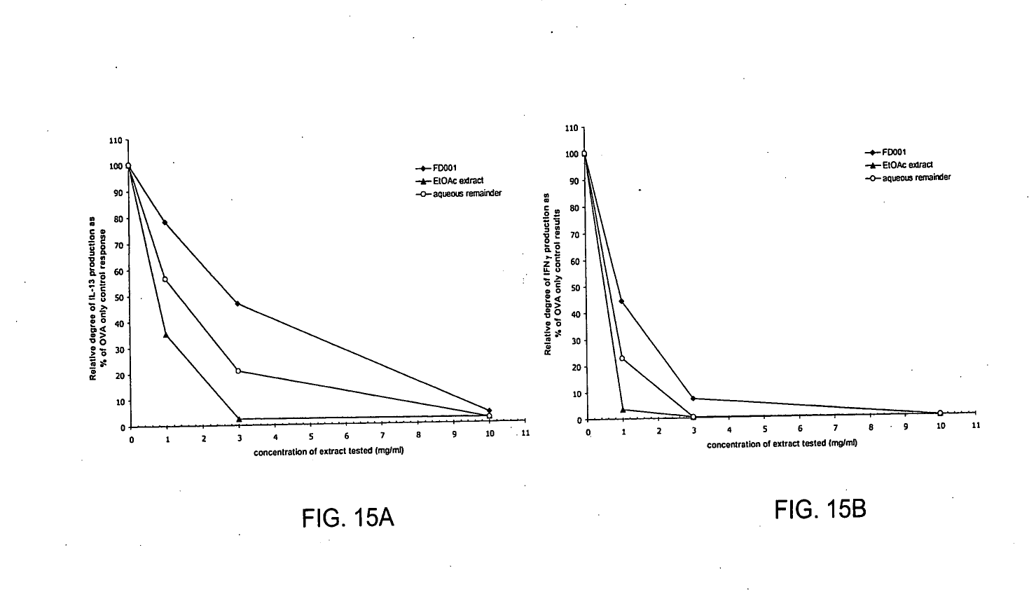

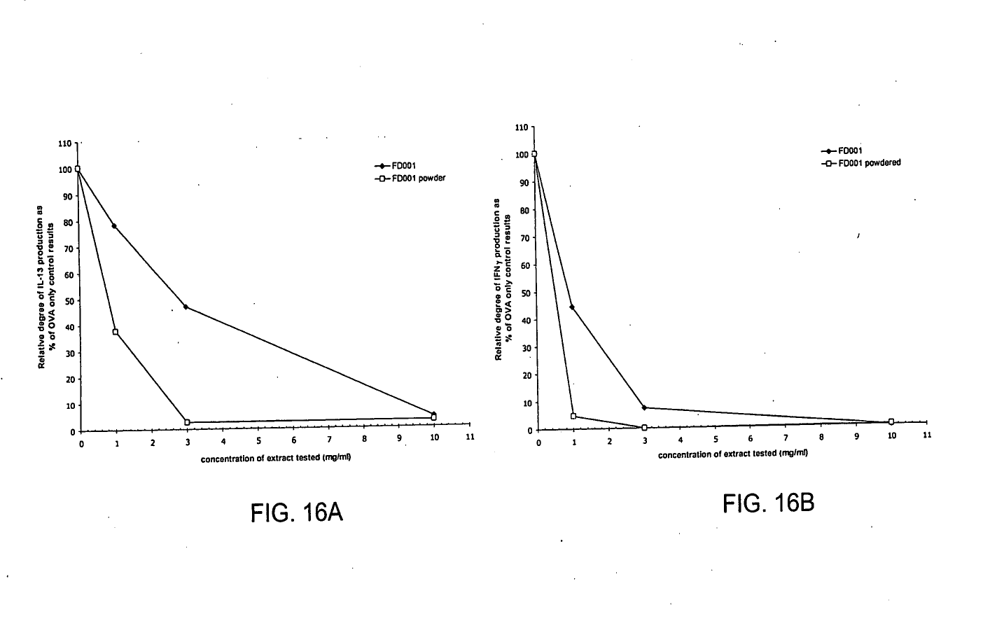

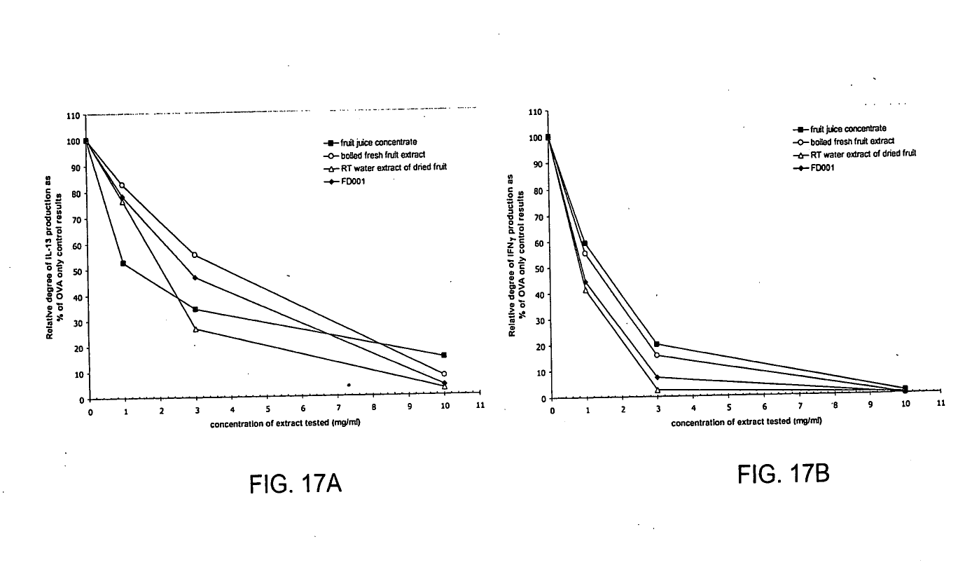

As demonstrated earlier, a greater suppression of the cytokines examined was observed as the concentrations of the A. arguta test materials were increased. In general, suppression was more pronounced against IFNγ. The activity of prescribed immunosuppressant compounds was similar to the A. arguta preparations in this assay. The peptide cyclosporin and the glucocorticoid steroid dexamethasone exhibited potent activity (<1 μM) as shown in FIG. 14A. The flavonoid quercetin showed potent activity over a slightly higher concentration range (1-25 μM, FIG. 14B). Further confirmation of the ability of EtOAc to extract activity from FD001 is demonstrated in FIGS. 15A and 15B. Interestingly, activity was also observed to be present in the aqueous remainder, indicating that both polar and non-polar components may be responsible for the in vitro immunosuppressive effect. The FD001 powder, which was the material used in both canine and human clinical trials (described below), was confirmed to be active in this assay as shown in FIGS. 16A and 16B.

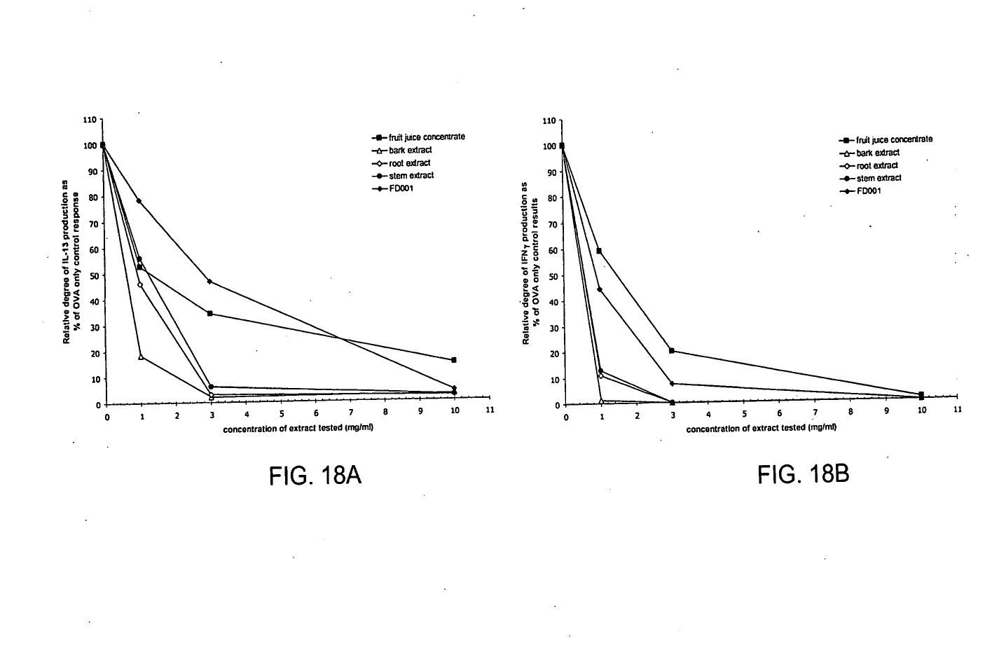

As described in Example 4, alternative methods of preparing the kiwifruit extracts, other than the procedures described in Examples 1 and 2, were explored. All of the fruit-derived extracts, whether dried or fresh, or extracted in hot or room temperature water, exhibited similar activity in this assay as seen in FIGS. 17A and 17B. Based on this analysis, the present inventors believe that there are several viable alternative methods for preparing kiwifruit for therapeutic purposes.

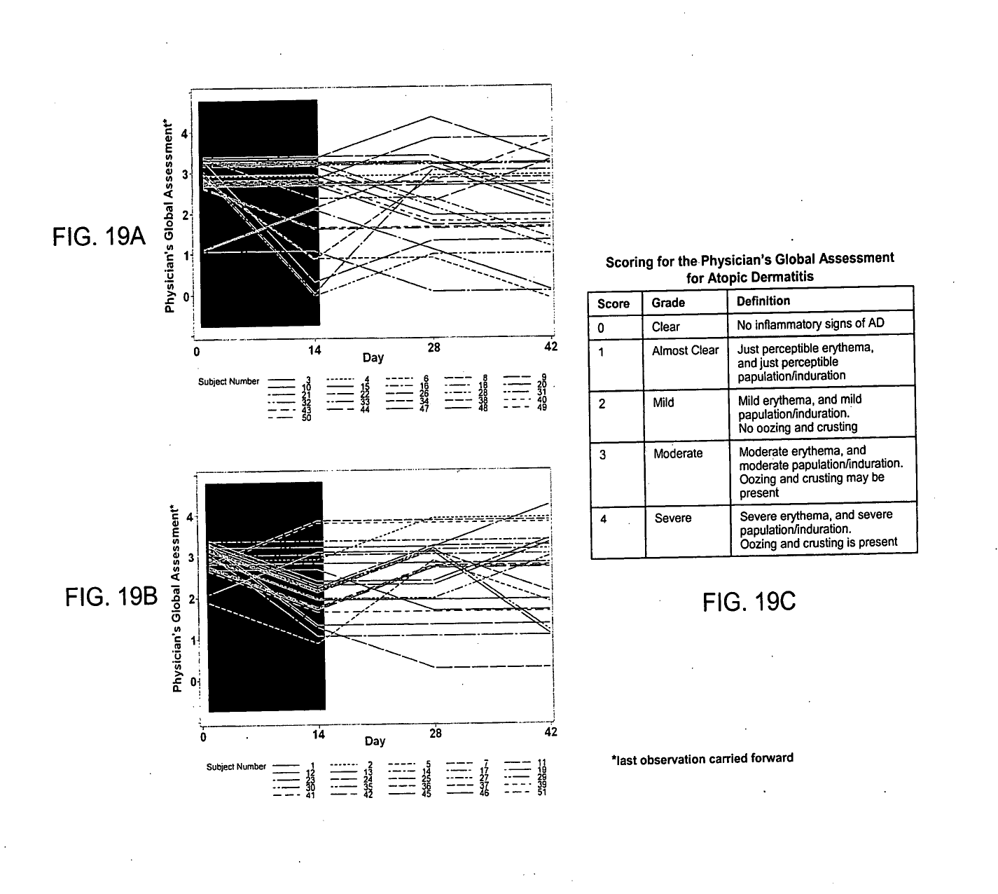

Interestingly, the hot water extracts prepared from the bark, root, and stem of A. arguta exhibited equal or superior activity in this assay (FIGS. 18A and 18B) when compared to FD001 (PG102T) and the concentrate of the fruit juice. These results indicate that these other plant parts may represent alternative sources of compounds of therapeutic interest with regard to modulation of immune markers or suppression of pro-inflammatory cytokines.

Each reference or publication described herein is incorporated herein by reference in its entirety except that U.S. patent application Ser. No. 11/945,153, U.S. patent application Ser. No. 11/697,565, U.S. patent application Ser. No. 11/518,380, U.S. patent application Ser. No. 11/362,606, U.S. Provisional Application No. 60/656,839 and U.S. Provisional Application No. 60/656,838 are not incorporated herein.

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following exemplary claims:

1. A method to regulate an immune response in a mammal, comprising administering a hardy kiwifruit extract to the mammal in an amount sufficient to regulate an immune response in the mammal.

2. The method of claim 1, wherein the hardy kiwifruit extract is provided in an amount sufficient to regulate a Th2 and a Th1 immune response in the mammal.

3. The method of claim 1, wherein the hardy kiwifruit extract is provided in an amount sufficient to regulate the amount of an antibody isotype produced by the mammal selected from the group consisting of IgE, IgG2a, and IgG1.

4. The method of claim 1, wherein the hardy kiwifruit extract is provided in an amount sufficient to decrease the production and/or levels of at least one Th2 cytokine in the mammal or to increase the level of at least one Th1 cytokine in the mammal.

5. The method of claim 1, wherein the hardy kiwifruit extract is provided in an amount sufficient to decrease the level of or production of at least one leukotriene in the mammal.

6. The method of claim 1, wherein the hardy kiwifruit extract is provided in an amount sufficient to decrease the level of expression of a transcription factor selected from the group consisting of: GATA-3, T-bet and NFATc2 in the mammal.

7. The method of claim 1, wherein the mammal has or is at risk of developing a condition in which enhancement of a Th1 response and/or suppression of a Th2 response is desirable.

8. The method of claim 7, wherein the mammal has or is at risk of developing an allergic disease or non-allergic inflammatory disease.

9. The method of claim 8, wherein the allergic disease is regulated by leukotrienes.

10. The method of claim 8, wherein the allergic disease is selected from the group consisting of: asthma and dermatitis.

11. The method of claim 7, wherein the mammal has or is at risk of developing a viral infection.

12. The method of claim 7, wherein the mammal has or is at risk of developing a cancer.

13. The method of claim 1, wherein the hardy kiwifruit is selected from the group consisting of: Actinidia arguta, Actinidia kolomikta and Actinidia polygama.

14. The method of claim 1, wherein the hardy kiwifruit is Actinidia arguta.

15. The method of claim 1, wherein the hardy kiwifruit extract is prepared from a part of the hardy kiwifruit selected from the group consisting of: the leaf, the stem, the bark, the root, and any combination thereof.

16. The method of claim 1, wherein the hardy kiwifruit extract is prepared from the group consisting of: fresh fruit, crushed fruit, boiled fruit, cooked fruit, pressed fruit, and condensed fruit.

17. The method of claim 1, wherein the hardy kiwifruit extract is prepared from dried fruit.

18. The method of claim 1, wherein the hardy kiwifruit extract is produced by a method that includes a step of drying the fruit.

19. The method of claim 1, wherein the hardy kiwifruit extract is a hardy kiwifruit juice concentrate.

20. The method of claim 1, wherein the hardy kiwifruit extract is produced by extraction of fruit in water having a temperature of between 0° C. and about 80° C.

21. The method of claim 1, wherein the hardy kiwifruit extract is produced by extraction of fruit in room temperature water.

22. The method of claim 1, wherein the hardy kiwifruit extract is produced by direct extraction of a water soluble concentrate of hardy kiwifruit with ethyl acetate.

23. The method of claim 1, wherein the hardy kiwifruit extract is provided in a composition in an amount of between about 0.01% and about 95% by weight based on the total weight of the composition.

24. The method of claim 1, wherein the step of administering comprises administering the hardy kiwifruit extract with a carrier, adjuvant, or diluent to the mammal.

25. The method of claim 1, wherein the step of administering comprises providing the hardy kiwifruit extract to the mammal as a tablet, a powder, an effervescent tablet, an effervescent powder, a capsule, a liquid, a suspension, a granule or a syrup.

26. The method of claim 1, wherein the step of administering comprises providing the hardy kiwifruit extract to the mammal in a health food.

27. The method of claim 26, wherein the health food is selected from the group consisting of: fine bakery wares, bread, rolls, breakfast cereals, processed cheese, unprocessed cheese, condiments, dairy products, puddings, gelatin desserts, carbonated drinks, teas, powdered beverage mixes, processed fish products, fruit-based drinks, vegetable-based drinks, chewing gum, hard confectionery, frozen dairy products, processed meat products, nut-based spreads, pasta, processed poultry products, gravies and sauces, potato chips, vegetable chips, crisps, chocolate, cookies, candy, licorice, ice creams, dehydrated foods, cut food products, processed food products, spices, alcoholic beverages, noodles, fermented foods, soups, soup mixes, soya based products, vegetable oil-based spreads, and vegetable-based drinks.

28. The method of claim 1, wherein the step of administering comprises applying a cosmetic composition comprising the hardy kiwifruit extract to the mammal.

29. The method of claim 28, wherein the cosmetic composition is provided in a form selected from the group consisting of: lotion, cream, essence, toner, emulsion, pack, soap, shampoo, rinse, cleanser, body washing solution, washing solution or treatment.

30. The method of claim 1, wherein the step of administering comprises providing the hardy kiwifruit extract to the mammal in a food additive.

31. The method of claim 1, further comprising administering to the mammal an agent selected from the group consisting of: fatty acids; polyketides; organic acids; small organic compounds; aromatic amino acids; phenylpropanoids; terpenoids; alkaloids; corrins; porphyrins; linear peptides; cyclic peptides; depsipeptides; amino acid derivatives; nucleosides; nucleotides; carbohydrates; proteins; cells; cell fragments; herbal preparations; spices; minerals; sterilizers; seasonings; vitamins; and electrolytes.

32. The method of claim 1, further comprising administering to the mammal an agent selected from the group consisting of: probiotics; bacterial cell walls and fragments; whey protein; taurine; alanine; fatty acids; fatty acid esters; monoglycerides; diglycerides; triglycerides; inositol; turmeric; curcumin; rosemary; rosemarinic acid; methylsulfonylmethane (MSM); ginseng; ginger; proanthocyanidin; and β-carotene.

33. The method of claim 32, wherein the fatty acids are selected from the group consisting of: conjugated linolenic acid, eicosapentaenoic acid, docosahexaenoic acid, γ-linolenic acid, α-linolenic acid, dihomo-γ-linolenic acid, and stearidonic acid.

34. The method of claim 1, further comprising administering to the mammal a different Actinidia species extract.

35. The method of claim 34, wherein the different Actinidia species is selected from the group consisting of: A. chenensis, A deliciosa, A. arguta, A. polygama, and A. kolomikta.

36. A composition for regulating an immune response in a mammal, comprising a hardy kiwifruit extract and at least one additional active compound for regulating an immune response in a mammal.

37. The composition of claim 36, wherein the additional active compound is for treating or preventing allergic disease in a mammal.

38. The composition of claim 36, wherein the additional active compound is selected from the group consisting of: antihistamines, antibodies, antibiotics, cyclosporins, antimycotics, respiratory function controllers, analgesics, β-agonists, leukotriene modifiers, cytokine or cytokine receptor antagonists, phosphodiesterase inhibitors, sodium cromoglycate, nedocrimil, caffeine, theophylline, carbobenzoxy beta-alanyl taurine, and inhibitors of T cell function.

39. The composition of claim 36, wherein the additional active compound is selected from the group consisting of: fatty acids; polyketides; citric acid; fumaric acid; guaiacol; methylsulfonylmethane (MSM); aromatic amino acids; phenylpropanoids; terpenoids; alkaloids; corrins; porphyrins; linear peptides; cyclic peptides; depsipeptides; amino acids derivatives; nucleosides; nucleotides; carbohydrates; proteins; cells; cell fragments; herbal preparations; spices; minerals; sterilizers; seasonings; vitamins; and electrolytes.

40. The composition of claim 36, wherein the additional active compound is selected from the group consisting of: probiotics; bacterial cell walls and fragments; whey protein; taurine; alanine; fatty acids; fatty acid esters; monoglycerides; diglycerides; triglycerides; inositol; turmeric; curcumin; rosemary; rosemarinic acid; methylsulfonylmethane (MSM); ginseng; ginger; proanthocyanidin; and β-carotene.

41. The composition of claim 40, wherein the fatty acids are selected from the group consisting of: conjugated linolenic acid, eicosapentaenoic acid, docosahexaenoic acid, γ-linolenic acid, α-linolenic acid, dihomo-γ-linolenic acid, and stearidonic acid.

42. The composition of claim 36, wherein the composition is selected from the group consisting of: a pharmaceutical composition, a health food, a food additive, and a cosmetic.

43. The composition of claim 36, wherein the hardy kiwifruit is selected from the group consisting of: Actinidia arguta, Actinidia kolomikta and Actinidia polygama.

44. The composition of claim 36, wherein the hardy kiwifruit is Actinidia arguta.

45. The composition of claim 36, wherein the hardy kiwifruit extract is prepared from a part of the hardy kiwifruit selected from the group consisting of: the fruit, the leaf, the stem, the bark, the root, and any combination thereof.

46. The composition of claim 36, wherein the hardy kiwifruit extract is prepared from the group consisting of: fresh fruit, crushed fruit, boiled fruit, cooked fruit, pressed fruit, and condensed fruit.

47. The composition of claim 36, wherein the hardy kiwifruit extract is prepared from dried fruit.

48. The composition of claim 36, wherein the hardy kiwifruit extract is produced by a method that includes a step of drying the fruit.

49. The composition of claim 36, wherein the hardy kiwifruit extract is a hardy kiwifruit juice concentrate.

50. The composition of claim 36, wherein the hardy kiwifruit extract is produced by extraction of fruit in water having a temperature of between about 0° C. and about 80° C.

51. The composition of claim 36, wherein the hardy kiwifruit extract is produced by extraction of fruit in room temperature water.

52. The composition of claim 36, wherein the hardy kiwifruit extract is produced by direct extraction of a water soluble concentrate of hardy kiwifruit with ethyl acetate.

53. The composition of claim 36, wherein the extract is prepared by extraction of hardy kiwifruit in distilled water.

54. The composition of claim 36, wherein the extract is an ethyl acetate extract of the hardy kiwifruit.

55. A method to treat a disease or condition that is associated with dysregulation of immune function, comprising administering to a mammal a combination of hardy kiwifruit extract and an agent selected from the group consisting of: an antihistamine, an antibody, an antibiotic, a cyclosporin, an antimycotic, a respiratory function controller, an analgesic, a β-agonists, a leukotriene modifier, a cytokine antagonist, a cytokine receptor antagonist, a phosphodiesterase inhibitor, sodium cromoglycate, nedocrimil, caffeine, theophylline, carbobenzoxy beta-alanyl taurine, and an inhibitor of T cell function.

56. The method of claim 55, wherein the fatty acids are selected from the group consisting of: conjugated linolenic acid, eicosapentaenoic acid, docosahexaenoic acid, γ-linolenic acid, α-linolenic acid, dihomo-γ-linolenic acid, and stearidonic acid.

57. The method of claim 55, wherein the composition is for the treatment of a disease or condition that is associated with leukotriene production or activity.

58. The method of claim 55, wherein the disease or condition is selected from the group consisting of: atopic dermatitis, asthma, food allergy, allergic rhinitis, and chronic urticaria.

59. The method of claim 55, wherein the hardy kiwifruit extract is selected from the group consisting of: a fruit extract, a leaf extract, a stem extract, a bark extract, a root extract, fresh fruit extract, crushed fruit extract, boiled fruit extract, cooked fruit extract, pressed fruit extract, condensed fruit extract, dried fruit extract, a hardy kiwifruit juice concentrate, an extract produced by extraction of fruit in water having a temperature of from 0° C. to about 80° C.; an extract produced by direct extraction of a water soluble concentrate of hardy kiwifruit with ethyl acetate, an extract produced by extraction of hardy kiwifruit in distilled water, and an extract produced by sequential extraction of hardy kiwifruit in water, chloroform and ethyl acetate.

60. A method to treat a disease or condition that is associated with dysregulation of immune function, comprising administering to a mammal a combination of a hardy kiwifruit extract and an agent selected from the group consisting of: fatty acids; polyketides; citric acid; fumaric acid; guaiacol; methylsulfonylmethane (MSM); aromatic amino acids; phenylpropanoids; terpenoids; alkaloids; corrins; porphyrins; linear peptides; cyclic peptides; depsipeptides; amino acid derivatives; nucleosides; nucleotides; carbohydrates; proteins; cells; cell fragments; herbal preparations; spices; minerals; sterilizers; seasonings; vitamins; and electrolytes.

61. The method of claim 60, wherein the fatty acids are selected from the group consisting of: conjugated linolenic acid, eicosapentaenoic acid, docosahexaenoic acid, γ-linolenic acid, α-linolenic acid, dihomo-γ-linolenic acid, and stearidonic acid.

62. The method of claim 60, wherein the composition is for the treatment of a disease or condition that is associated with leukotriene production or activity.

63. The method of claim 60, wherein the disease or condition is selected from the group consisting of: atopic dermatitis, asthma, food allergy, allergic rhinitis, and chronic urticaria.

64. The method of claim 60, wherein the hardy kiwifruit extract is selected from the group consisting of: a fruit extract, a leaf extract, a stem extract, a bark extract, a root extract, fresh fruit extract, crushed fruit extract, boiled fruit extract, cooked fruit extract, pressed fruit extract, condensed fruit extract, dried fruit extract, a hardy kiwifruit juice concentrate, an extract produced by extraction of fruit in water having a temperature of from 0° C. to about 80° C.; an extract produced by direct extraction of a water soluble concentrate of hardy kiwifruit with ethyl acetate, an extract produced by extraction of hardy kiwifruit in distilled water, and an extract produced by sequential extraction of hardy kiwifruit in water, chloroform and ethyl acetate.

65. A method to treat a disease that is associated with dyregulation of immune function, comprising administering to a mammal a combination of hardy kiwifruit extract and an agent selected from the group consisting of: probiotics; bacterial cell walls and fragments; whey protein; taurine; alanine, fatty acids; fatty acid esters; monoglycerides; diglycerides; triglycerides; inositol; turmeric; curcumin; rosemary; rosemarinic acid; methylsulfonylmethane (MSM); ginseng; ginger; proanthocyanidin; and β-carotene.

66. The method of claim 65, wherein the composition is for the treatment of a disease or condition that is associated with leukotriene production or activity.

67. The method of claim 65, wherein the disease or condition is selected from the group consisting of: atopic dermatitis, asthma, food allergy, allergic rhinitis, and chronic urticaria.

68. The method of claim 65, wherein the hardy kiwifruit extract is selected from the group consisting of: a fruit extract, a leaf extract, a stem extract, a bark extract, a root extract, fresh fruit extract, crushed fruit extract, boiled fruit extract, cooked fruit extract, pressed fruit extract, condensed fruit extract, dried fruit extract, a hardy kiwifruit juice concentrate, an extract produced by extraction of fruit in water having a temperature of from 0° C. to about 80° C.; an extract produced by direct extraction of a water soluble concentrate of hardy kiwifruit with ethyl acetate, an extract produced by extraction of hardy kiwifruit in distilled water, and an extract produced by sequential extraction of hardy kiwifruit in water, chloroform and ethyl acetate.
